(19) United States
(12) Patent Application Publication
(10) Pub. No.: US 2017/0174826 A1
(43) Pub. Date: Jun. 22, 2017

(54) DONOR-ACCEPTOR POLYMERS

(71) Applicant: AGENCY FOR SCIENCE, TECHNOLOGY AND RESEARCH, Singapore (SG)

(72) Inventors: Qun YE, Singapore (SG); Ching Mui CHO, Singapore (SG); Wei Teng NEO, Singapore (SG); Jianwei XU, Singapore (SG)

(73) Assignee: AGENCY FOR SCIENCE, TECHNOLOGY AND RESEARCH, Singapore (SG)

(21) Appl. No.: 15/327,026

(22) PCT Filed: Jul. 16, 2015
(86) PCT No.: PCT/SG2015/050218

§ 371 Date: Jan. 17, 2017

(30) Foreign Application Priority Data
Jul. 16, 2014 (SG) 10201404150Q

(51) Int. Cl. C08G 61/12; H01L 51/00; C07D 487/04

(52) U.S. Cl. CPC C08G 61/126; C07D 487/04; C08G 61/122; C08G 61/125; H01L 51/0043; H01L 51/0036; H01L 51/0039; C08G 2261/122; C08G 2261/143; C08G 2261/1412; C08G 2261/1424; C08G 2261/3223; C08G 2261/3221; C08G 2261/3243; C08G 2261/3142; C08G 2261/3241; C08G 2261/3222; C08G 2261/414; C08G 2261/95; C08G 2261/92; C08G 2261/91; H01L 51/0512

(57) ABSTRACT

The present invention relates to compounds that are electron-deficient acceptors, donor-acceptor polymers that are synthesized from such electron-deficient acceptors, semiconductor devices synthesized from such donor-acceptor polymers and methods of synthesis of such electron-deficient acceptors, donor-acceptor polymers and semiconductor devices.

### TECHNICAL FIELD

The present invention generally relates to compounds that may be used as building blocks to construct donor-acceptor polymers. More particularly, the present invention relates to electron-deficient imide-fused pyridazines and imide-fused phthalazines. The present invention also relates to the donor-acceptor polymers constructed using said building blocks and methods for preparing such building blocks and donor-acceptor polymers.

### BACKGROUND ART

Organic electronics materials have attracted attention due to their potential in applications such as organic field effect transistors, organic light emitting diodes, organic photovoltaic cells, organic electrochromic materials and sensor materials. However, the commercialization of such devices have been slow due to the lack of suitable materials having high stability, long shelf-life, high performance and a low cost of production.

Donor-acceptor type conjugated oligomers and polymers have been investigated as organic electronics materials. The physical and optical properties of donor-acceptor type conjugated oligomers and polymers can be conveniently tuned by adjustment of their donor and acceptor moieties.

In many applications of organic electronics materials, the electron affinity of the acceptor building blocks is of great importance in tuning the physical and optical properties of the materials and in most cases is crucial to assure the required performance characteristics of the material. The electron affinity plays a vital role in controlling the band-gap of the resultant conjugated polymers. In order to make low band-gap conjugated polymers with desired absorption characteristics for opto-electronic applications, more electron-deficient acceptors especially with high electron deficiency are needed to manufacture organic electronics materials.

There is therefore a need to provide building blocks that overcome, or at least ameliorate, one or more of the disadvantages described above. There is also a need to provide donor-acceptor polymers produced by these building blocks, which may be used as organic electronics materials that overcome, or at least ameliorate, one or more of the disadvantages described above. There is also a need to provide a method of synthesizing building blocks and donor-acceptor polymers that overcome, or at least ameliorate, one or more of the disadvantages described above.

### SUMMARY

In a first aspect, there is provided a compound having the following Formula (I);

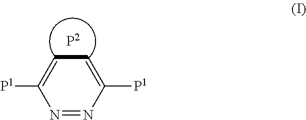

wherein P¹ has the following Formula (IIa) or (IIb);

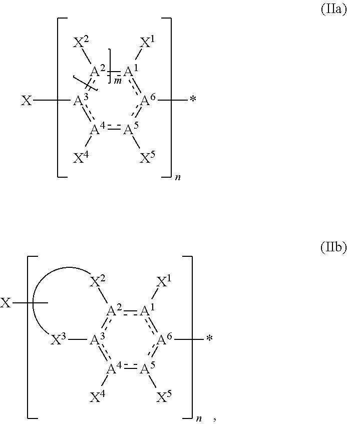

wherein
[symbol] is a single or double bond, as valency allows;
m is 0 or 1;
n is an integer selected from 1 to 4;
X, X¹, X², X³, X⁴ and X⁵ are independently absent or selected from the group consisting of a bond, H, halogen, CN, R¹, OR¹ or SR¹;
wherein R¹ is selected from the group consisting of optionally substituted alkyl, optionally substituted alkenyl, optionally substituted alkynyl, optionally substituted cycloalkyl, optionally substituted heterocycloalkyl, optionally substituted cycloalkenyl, optionally substituted heterocycloalkenyl, optionally substituted aryl, optionally substituted heteroaryl,
A¹ is selected from the group consisting of O, S, Se, Te, C and N
A², A³, A⁴, A⁵ and A⁶ are independently C or N; and
wherein any two substituents selected from the group consisting of X¹, X², X³, X⁴ and X⁵ may optionally be taken together to form an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl;
and wherein P² has the following formula (IIIa) or (IIIb);

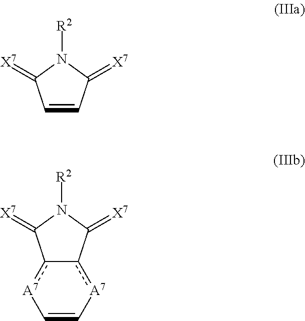

wherein
R² is an optionally substituted alkyl or optionally substituted aryl;
X⁷ is O or S;
A⁷ is CH, CH₂ or N;
and [symbol] is where the compound of Formula (IIIa) or (IIIb) is fused to the compound of Formula (I).

Advantageously, the compound of Formula (I) as defined above may combine an electron withdrawing imide group to an electron deficient pyridazine or phthalazine group, resulting in the compound having ultra-high electron affinity. The compound of Formula (I) as defined above may be used as electron-deficient acceptors with unprecedentedly high electron affinity. The compound of Formula (I) as defined above may be even more electron deficient compared to conventional compounds. Further advantageously, the compound of Formula (I) may be easy to functionalize, allowing for fine-tuning of not only its electronic properties but also to enhance its processability. For example, the compound of Formula (I) as defined above may be easily functionalized to improve its solubility. More advantageously, the easily functionalizable nature of the compound of Formula (I) may facilitate its integration with donor compounds. This may be useful, for example, in the manufacture of more processable polymers which may further be used as organic electronic materials.

In many applications of organic electronics materials, the electron affinity of the building blocks may be of great importance in tuning the physical and optical properties of the materials and in most cases may be crucial to ensure that the resulting material has the correct characteristics. The electron affinity may play a vital role in controlling the band-gap of the resultant conjugated polymers formed from the electron deficient pyridazine or phthalazine group. Advantageously, by attaching an electron withdrawing imide group to an electron deficient pyridazine or phthalazine group, a compound of Formula (I), which may possess ultra-high electron affinity, may be formed. This building block defined by Formula (I) may be designed to be easily functionalizable and therefore may be of high importance for the development of organic electronics materials. More advantageously, the compound of Formula (I) may be synthesized with a good overall synthetic yield. Further, the compound of Formula (I) may be easily incorporated into donor-acceptor conjugated polymers.

In a second aspect, there is provided a compound having repeating units of the following Formula (IV);

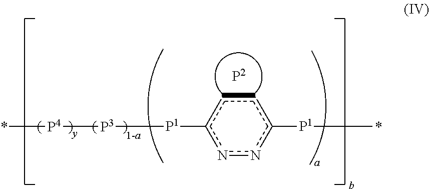

wherein P¹ has the following Formula (IIa) or (IIb);

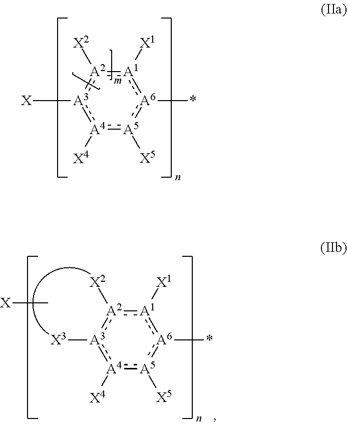

wherein
[symbol] is a single or double bond, as valency allows;
m is 0 or 1;
n is an integer selected from 1 to 4;
X¹, X², X³, X⁴ and X⁵ are independently absent or selected from the group consisting of a bond, H, halogen, CN, R¹, OR¹ or SR¹;
wherein R¹ is selected from the group consisting of optionally substituted alkyl, optionally substituted alkenyl, optionally substituted alkynyl, optionally substituted cycloalkyl, optionally substituted heterocycloalkyl, optionally substituted cycloalkenyl, optionally substituted heterocycloalkenyl, optionally substituted aryl, optionally substituted heteroaryl,
A¹ is selected from the group consisting of O, S, Se, Te, C and N
A², A³, A⁴, A⁵ and A⁶ are independently C or N; and
wherein any two substituents selected from the group consisting of X¹, X², X³, X⁴ and X⁵ may optionally be taken together to form an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl;
P² has the following formula (IIIa) or (IIIb);

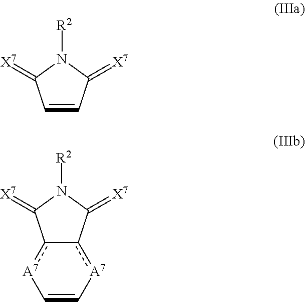

wherein
R² is an optionally substituted alkyl or optionally substituted aryl;
X⁷ is O or S;
A⁷ is CH, CH₂ or N;
and [symbol] is where the compound of Formula (IIIa) or (IIIb) is fused to the compound of Formula (IV);
P³ has the following Formula (Va), (Vb) or (Vc);

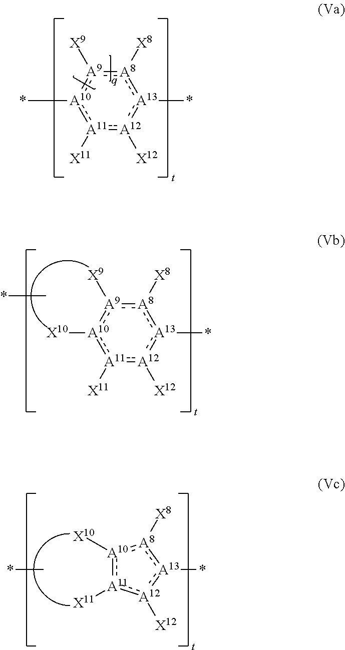

wherein
q is 0 or 1;
t is an integer selected from 1 to 4;
X⁸, X⁹, X¹¹ and X¹² are independently absent or selected from the group consisting of a bond, H, halogen, CN, R³, OR³ or SR³;
wherein R3 is selected from the group consisting of optionally substituted alkyl, optionally substituted alkenyl, optionally substituted alkynyl, optionally substituted cycloalkyl, optionally substituted heterocycloalkyl, optionally substituted cycloalkenyl, optionally substituted heterocycloalkenyl, optionally substituted aryl, optionally substituted heteroaryl,
A⁸ is selected from the group consisting of O, S, Se, Te, C and N
A⁹, A¹⁰, A¹¹, A¹² and A¹³ are independently C or N;
wherein any two substituents selected from the group consisting of X⁸, X⁹, X¹⁰, X¹¹ and X¹² may optionally be taken together to form an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl;
and P⁴ is absent, or an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl. a is any value from 0 to 1; and
b is an integer selected from 1 to 10,000; and
y is an integer selected from 0 to 5.

Advantageously, the compound of Formula (IV) may be a polymer. The purification of a polymer may be much easier and more convenient than purification of small molecules. Advantageously, the simple purification process may significantly reduce the cost of production of the polymer. Further advantageously, the polymer may have good solubility in a variety of solvents. The polymer may be soluble in non-chlorinated solvents, making the polymer easily processable in solution. This may further reduce the cost of production. More advantageously, the polymer may become glassy upon heating and this may be useful in manufacturing devices using the polymer material.

Further advantageously, the polymer may be a donor-acceptor polymer. Advantageously, the polymer may be comprised of donor compounds and acceptor compounds. Further advantageously, the donor compounds and acceptor compounds may be selected to confer advantageous properties to the polymer. Advantageously, the acceptor compound in the polymer may be the compound of Formula (I) as defined above. The ultra-electron deficient nature of the compound of Formula (I) as defined above may enable ease of synthesis of the polymer as well as confer advantageous properties to the polymer. Further advantageously, the pyridazine or phthalazine group of the compound of Formula (I) may lower the frontier orbitals of the polymers to make the polymer more stable under ambient conditions.

The performance of donor-acceptor type conjugated oligomers and polymers may rely on many factors, including the identity of the donor and acceptor building block of the polymer, the polymerization strategy and the molar weight of the polymer. Advantageously, the compound of Formula (IV) as defined above has been synthesized by judicious choice of the donor and acceptor building block, polymerization strategy and molecular weight of the polymer to so that the polymer has the advantageous properties such as good band-gap, good optical property, high stability and low cost of production.

In a third aspect, there is provided a compound having the following Formula (VI);

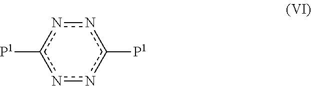

wherein P¹ has the following Formula (IIa) or (IIb);

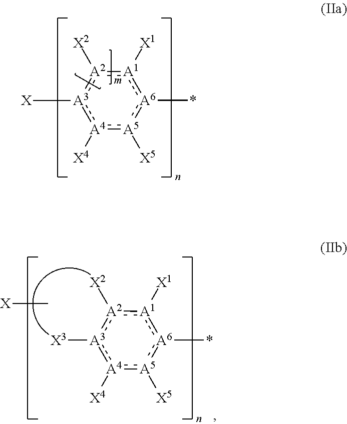

wherein
[symbol] is a single or double bond, as valency allows;
m is 0 or 1;
n is an integer selected from 1 to 4;
X, X¹, X², X³, X⁴ and X⁵ are independently absent or selected from the group consisting of a bond, H, halogen, CN, R¹, OR¹ or SR¹;
wherein R¹ is selected from the group consisting of optionally substituted alkyl, optionally substituted alkenyl, optionally substituted alkynyl, optionally substituted cycloalkyl, optionally substituted heterocycloalkyl, optionally substituted cycloalkenyl, optionally substituted heterocycloalkenyl, optionally substituted aryl, optionally substituted heteroaryl,
A¹ is selected from the group consisting of O, S, Se, Te, C and N;
A², A³, A⁴, A⁵ and A⁶ are independently C or N; and wherein any two substituents selected from the group consisting of X¹, X², X³, X⁴ and X⁵ may optionally be taken together to form an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl.

Advantageously, the compound of Formula (I) as defined above may be a useful starting material for the synthesis of compounds defined further below. Advantageously, the compound of Formula (I) as defined above may comprise a tetrazine moiety attached to two electron donating groups. Advantageously, the tetrazine moiety may be electron-rich, such that when reacted with a dienophile defined further below, the resulting product will have ultra-high electron affinity.

In a fourth aspect, there is provided a compound having the following formula (VIIa) or (VIIb);

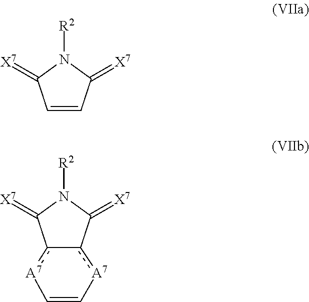

wherein
[symbol] is a single or double bond, as valency allows;
R² is an optionally substituted alkyl or optionally substituted aryl;
X⁷ is O or S; and
A⁷ is CH, CH₂ or N.

Advantageously, the compounds of formula (VIIa) or (VIIb) as defined above may be intrinsically electron-withdrawing, such that when reacted with the tetrazine compound having Formula (VI) as defined above, the resulting product will have ultra-high electron affinity. Further advantageously, the compounds of Formula (VIIa) or (VIIb) may be easy to functionalize. More advantageously, the compound of Formula (VIIa) or (VIIb) as defined above may be tunable by their R² functional group to enhance their solubility properties such that they may be useful in reactions performed in a variety of solvents.

In a fifth aspect, there is provided a method for synthesizing the compound of Formula (VI) as defined above, comprising the steps of providing a hydrazine and contacting the hydrazine with a functionalized cyclic group, wherein the cyclic group of the functionalized cyclic group is selected from the group comprising of optionally substituted furan, optionally substituted thiophene, optionally substituted selenophene, optionally substituted tellurophene, optionally substituted pyrrole, optionally substituted phenylene, optionally substituted aza-phenylene, optionally substituted arylene diimide, optionally substituted fluorene, optionally substituted cabazole, optionally substituted dibenzodilole, optionally substituted benzooxodiazole, optionally substituted benzotriazole, optionally substituted benzothidiazole and any combination thereof.

In a sixth aspect, there is provided a method for synthesizing the compound of Formula (I) as defined above, comprising the steps of:
providing the compound of Formula (VI) of claim 3; and
contacting the compound of Formula (VI) of claim 3 with the compound of Formulae (VIIa) or (VIIb) of claim 4 under reaction conditions to form the compound of Formula (I) of claim 1.

Advantageously, the method for synthesizing the compound of Formula (I) as defined above may be an inverse-electron demanding Diels-Alder reaction. Conventionally, for Diels-Alder reactions that are being carried out on a tetrazine, the tetrazine acts as a diene and therefore should be modified with electron withdrawing groups to enhance its reactivity. Conversely, the dienophile should be modified with electron donating groups. This is to facilitate the inverse electron demanding Diels-Alder reaction. However, in the synthesis of the compound of Formula (I) as defined above, the tetrazine moiety has two electron donating units PI attached. In addition, the dienophile (compounds of Formulae (VIIa) or (VIIb) as defined above) is an intrinsically electron withdrawing imide. Under such conditions, the inverse-electron demanding Diels-Alder reaction has conventionally faced challenges due to the unfavourable electronic interaction between the tetrazine and the dienophile.

However, advantageously, the compound of Formula (I) may be synthesized with a good overall yield by the method as defined above. More advantageously, the successful synthesis may be due to judicious choice of reaction solvents, reaction temperature and reaction duration. Advantageously, the solvent used for the reaction may not contain any reactivity towards the reaction mixture. More advantageously, the boiling point of the solvent may be sufficiently high to allow the reaction to proceed. Advantageously, the method as defined above may enable the synthesis of the compound of Formula (I) with an isolated yield of higher than 90%.

Further advantageously, the method as defined above is scalable. The compound of Formula (I) as defined above may therefore be synthesized at a larger scale of up to 500 g.

In an seventh aspect, there is provided a method for synthesizing the compound of Formula (IV) as defined above, comprising the step of:
providing the compound of Formula (I) of claim 1; and
contacting the compound of Formula (I) of claim 1 with;
   (i) a compound having the following Formula (XXVa), (XXVb) or (XXVc);

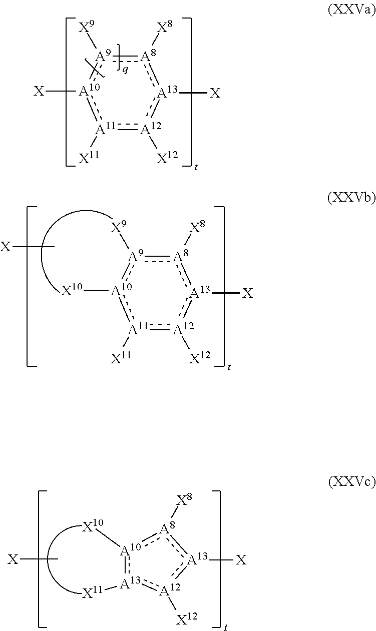

wherein
[symbol] is a single or double bond, as valency allows;
q is 0 or 1;
t is an integer selected from 1 to 4;
X, X⁸, X⁹, X¹¹ and X¹² are independently absent or selected from the group consisting of a bond, H, halogen, CN, R³, OR³ or SR³;
wherein R³ is selected from the group consisting of optionally substituted alkyl, optionally substituted alkenyl, optionally substituted alkynyl, optionally substituted cycloalkyl, optionally substituted heterocycloalkyl, optionally substituted cycloalkenyl, optionally substituted heterocycloalkenyl, optionally substituted aryl, optionally substituted heteroaryl,
A⁸ is selected from the group consisting of O, S, Se, Te, C and N
A⁹, A¹⁰, A¹¹, A¹² and A¹³ are independently C or N; and
wherein any two substituents selected from the group consisting of X⁸, X⁹, X¹⁰, X¹¹ and X¹² may optionally be taken together to form an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl; and
   (ii) a cross-coupling reagent comprising a cyclic group; and an element selected from the group consisting of zinc, tin and boron; wherein the cyclic group is selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl;
in the presence of a metal-containing catalyst.

In an eighth aspect, there is provided a method for synthesizing the compound of Formula (IV) as defined above, comprising the steps of:
1) providing the compound of Formula (I) of claim 1; and
2) contacting the compound of Formula (I) of claim 1 with a compound having the following Formula (XXVI);

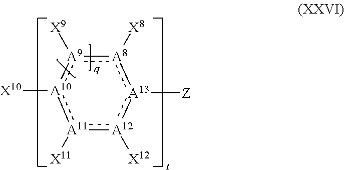

wherein
[symbol] is a single or double bond, as valency allows;
q is 0 or 1;
t is an integer selected from 1 to 4;
Z is a functional group containing zinc, tin or born;
X⁸, X⁹, X¹⁰, X¹¹ and X¹² are independently absent or selected from the group consisting of a bond, H, halogen, CN, R³, OR³ or SR³;
wherein R³ is selected from the group consisting of optionally substituted alkyl, optionally substituted alkenyl, optionally substituted alkynyl, optionally substituted cycloalkyl, optionally substituted heterocycloalkyl, optionally substituted cycloalkenyl, optionally substituted heterocycloalkenyl, optionally substituted aryl, optionally substituted heteroaryl,
A⁸ is selected from the group consisting of O, S, Se, Te, C and N
A⁹, A¹⁰, A¹¹, A¹² and A¹³ are independently C or N; and
wherein any two substituents selected from the group consisting of X⁸, X⁹, X¹⁰, X¹¹ and X¹² may optionally be taken together to form an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl;
in the presence of a metal-containing catalyst to form a monomer precursor;
3) reacting the monomer precursor of step (2) with N-bromosuccinimide to form a reactive monomer; and
4) reacting the reactive monomer of step (2) with a cross-coupling reagent comprising a cyclic group; and an element selected from the group consisting of zinc, tin and boron; wherein the cyclic group is selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl;
in the presence of a metal-containing catalyst.

Advantageously, the method as defined above may have a high yield of synthesis. Further advantageously, the method as defined above may facilitate a controlled polymerization of the monomers, giving rise to polymers with defined ratios of donors and acceptors.

In a ninth aspect, there is provided the use of the compound of Formula (IV) as defined above, in the manufacture of semiconductor devices.

In a tenth aspect, there is provided a semiconductor device comprising the compound of Formula (IV) as defined above.

Advantageously, the compound of Formula (IV) as defined above may be useful in the manufacture of semiconductor devices. More advantageously, the compound of Formula (IV) as defined above may have enhanced processability, enabling them to be used in the manufacture of more processable polymers. Advantageously, the semiconductor devices manufactured using the compound of Formula (IV) as defined above may be used in a wide range of applications such as electrochromic materials, organic light-emitting diode, organic thin-film transistors and organic photovoltaic cells. Advantageously, the semiconductor devices manufactured using the compound of Formula (IV) may have higher stability, long shelf-life, high performance and may be cheaper to manufacture. Further advantageously, the device comprising the compound of Formula (IV) as defined above may have improved optical properties.

### DEFINITIONS

In this specification a number of terms are used which are well known to a skilled addressee. Nevertheless for the purposes of clarity a number of terms will be defined. The following words and terms used herein shall have the meaning indicated:

In the definitions of a number of substituents below it is stated that “the group may be a terminal group or a bridging group”. This is intended to signify that the use of the term is intended to encompass the situation where the group is a linker between two other portions of the molecule as well as where it is a terminal moiety. Using the term alkyl as an example, some publications would use the term “alkylene” for a bridging group and hence in these other publications there is a distinction between the terms “alkyl” (terminal group) and “alkylene” (bridging group). In the present application no such distinction is made and most groups may be either a bridging group or a terminal group.

“Acyl” means an R—C(═O)— group in which the R group may be an alkyl, cycloalkyl, heterocycloalkyl, aryl or heteroaryl group as defined herein. Examples of acyl include acetyl and benzoyl. The group may be a terminal group or a bridging group. If the group is a terminal group it is bonded to the remainder of the molecule through the carbonyl carbon.

“Acylamino” means an R—C(═O)—NH— group in which the R group may be an alkyl, cycloalkyl, heterocycloalkyl; aryl or heteroaryl group as defined herein. The group may be a terminal group or a bridging group. If the group is a terminal group it is bonded to the remainder of the molecule through the nitrogen atom.

“Alkenyl” as a group or part of a group denotes an aliphatic hydrocarbon group containing at least one carbon-carbon double bond and which may be straight or branched preferably having 2-12 carbon atoms, more preferably 2-10 carbon atoms, most preferably 2-6 carbon atoms, in the normal chain. The group may contain a plurality of double bonds in the normal chain and the orientation about each is independently E or Z. Exemplary alkenyl groups include, but are not limited to, ethenyl, propenyl, butenyl, pentenyl, hexenyl, heptenyl, octenyl and nonenyl. The group may be a terminal group or a bridging group.

“Alkenyloxy” refers to an alkenyl-O— group in which alkenyl is as defined herein. Preferred alkenyloxy groups are C₁-C₆ alkenyloxy groups. The group may be a terminal group or a bridging group. If the group is a terminal group it is bonded to the remainder of the molecule through the oxygen atom.

“Alkyl” as a group or part of a group refers to a linear or branched aliphatic hydrocarbon group, preferably a C₁-C₂₅ alkyl, more preferably a C₁-C₁₀ alkyl, most preferably C₁-C₆ unless otherwise noted. Examples of suitable straight and branched C₁-C₆ alkyl substituents include methyl, ethyl, n-propyl, 2-propyl, n-butyl, sec-butyl, t-butyl, hexyl, and the like. The group may be a terminal group or a bridging group.

“Alkylamino” includes both mono-alkylamino and dialkylamino, unless specified. “Mono-alkylamino” means a Alkyl-NH— group, in which alkyl is as defined herein. “Dialkylamino” means a (alkyl)₂N— group, in which each alkyl may be the same or different and are each as defined herein for alkyl. The alkyl group is preferably a C₁-C₆ alkyl group. The group may be a terminal group or a bridging group. If the group is a terminal group it is bonded to the remainder of the molecule through the nitrogen atom.

“Alkylaminocarbonyl” refers to a group of the formula (Alkyl)ₓ(H)_{y}NC(═O)— in which alkyl is as defined herein, x is 1 or 2, and the sum of X+Y=2. The group may be a terminal group or a bridging group. If the group is a terminal group it is bonded to the remainder of the molecule through the carbonyl carbon.

“Alkyloxy” refers to an alkyl-O— group in which alkyl is as defined herein. Preferably the alkyloxy is a C₁-C₆alkyloxy. Examples include, but are not limited to, methoxy and ethoxy. The group may be a terminal group or a bridging group.

“Alkyloxyalkyl” refers to an alkyloxy-alkyl-group in which the alkyloxy and alkyl moieties are as defined herein. The group may be a terminal group or a bridging group. If the group is a terminal group it is bonded to the remainder of the molecule through the alkyl group.

“Alkyloxyary” refers to an alkyloxy-aryl-group in which the alkyloxy and aryl moieties are as defined herein. The group may be a terminal group or a bridging group. If the group is a terminal group it is bonded to the remainder of the molecule through the aryl group.

“Alkyloxycarbonyl” refers to an alkyl-O—C(═O)— group in which alkyl is as defined herein. The alkyl group is preferably a C₁-C₆ alkyl group. Examples include, but are not limited to, methoxycarbonyl and ethoxycarbonyl. The group may be a terminal group or a bridging group. If the group is a terminal group it is bonded to the remainder of the molecule through the carbonyl carbon.

“Alkyloxycycloalkyl” refers to an alkyloxy-cycloalkyl-group in which the alkyloxy and cycloalkyl moieties are as defined herein. The group may be a terminal group or a bridging group. If the group is a terminal group it is bonded to the remainder of the molecule through the cycloalkyl group.

“Alkyloxyheteroaryl” refers to an alkyloxy-heteroaryl-group in which the alkyloxy and heteroaryl moieties are as defined herein. The group may be a terminal group or a bridging group. If the group is a terminal group it is bonded to the remainder of the molecule through the heteroaryl group.

“Alkyloxyheterocycloalkyl” refers to an alkyloxy-heterocycloalkyl-group in which the alkyloxy and heterocycloalkyl moieties are as defined herein. The group may be a terminal group or a bridging group. If the group is a terminal group it is bonded to the remainder of the molecule through the heterocycloalkyl group.

“Alkylsulfinyl” means an alkyl-S—(═O)— group in which alkyl is as defined herein. The alkyl group is preferably a C₁-C₆ alkyl group. Exemplary alkylsulfinyl groups include, but not limited to, methylsulfinyl and ethylsulfinyl. The group may be a terminal group or a bridging group. If the group is a terminal group it is bonded to the remainder of the molecule through the sulfur atom.

“Alkylsulfonyl” refers to an alkyl-S(═O)₂— group in which alkyl is as defined above. The alkyl group is preferably a C₁-C₆ alkyl group. Examples include, but not limited to methylsulfonyl and ethylsulfonyl. The group may be a terminal group or a bridging group. If the group is a terminal group it is bonded to the remainder of the molecule through the sulfur atom.

“Alkynyl” as a group or part of a group means an aliphatic hydrocarbon group containing a carbon-carbon triple bond and which may be straight or branched preferably having from 2-12 carbon atoms, more preferably 2-10 carbon atoms, more preferably 2-6 carbon atoms in the normal chain. Exemplary structures include, but are not limited to, ethynyl and propynyl. The group may be a terminal group or a bridging group.

“Alkynyloxy” refers to an alkynyl-O— group in which alkynyl is as defined herein. Preferred alkynyloxy groups are C₁-C₆ alkynyloxy groups. The group may be a terminal group or a bridging group. If the group is a terminal group it is bonded to the remainder of the molecule through the oxygen atom.

“Amino” refers to groups of the form —NRₐR_{b} wherein Rₐ and R_{b} are individually selected from the group including but not limited to hydrogen, optionally substituted alkyl, optionally substituted alkenyl, optionally substituted alkynyl, and optionally substituted aryl groups.

“Aminoalkyl” means an NH₂-alkyl-group in which the alkyl group is as defined herein. The group may be a terminal group or a bridging group. If the group is a terminal group it is bonded to the remainder of the molecule through the alkyl group.

“Aminosulfonyl” means an NH₂—S(═O)₂— group. The group may be a terminal group or a bridging group. If the group is a terminal group it is bonded to the remainder of the molecule through the sulfur atom.

“Aryl” as a group or part of a group denotes (i) an optionally substituted monocyclic, or fused polycyclic, aromatic carbocycle (ring structure having ring atoms that are all carbon) preferably having from 5 to 18 atoms per ring. Examples of aryl groups include phenyl, naphthyl, and the like; (ii) an optionally substituted partially saturated bicyclic aromatic carbocyclic moiety in which a phenyl and a C₅₋₇ cycloalkyl or C₅₋₇ cycloalkenyl group are fused together to form a cyclic structure, such as tetrahydronaphthyl, indenyl or indanyl. The group may be a terminal group or a bridging group. Typically an aryl group is a C₃-C₁₈ aryl group.

“Arylalkenyl” means an aryl-alkenyl-group in which the aryl and alkenyl are as defined herein. Exemplary arylalkenyl groups include phenylallyl. The group may be a terminal group or a bridging group. If the group is a terminal group it is bonded to the remainder of the molecule through the alkenyl group.

“Arylalkyl” means an aryl-alkyl-group in which the aryl and alkyl moieties are as defined herein. Preferred arylalkyl groups contain a C₁₋₅ alkyl moiety. Exemplary arylalkyl groups include benzyl, phenethyl, 1-naphthalenemethyl and 2-naphthalenemethyl. The group may be a terminal group or a bridging group. If the group is a terminal group it is bonded to the remainder of the molecule through the alkyl group.

“Arylalkyloxy” refers to an aryl-alkyl-O— group in which the alkyl and aryl are as defined herein. The group may be a terminal group or a bridging group. If the group is a terminal group it is bonded to the remainder of the molecule through the oxygen atom.

“Arylamino” includes both mono-arylamino and di-arylamino unless specified. Mono-arylamino means a group of formula arylNH—, in which aryl is as defined herein, di-arylamino means a group of formula (aryl)₂N— where each aryl may be the same or different and are each as defined herein for aryl. The group may be a terminal group or a bridging group. If the group is a terminal group it is bonded to the remainder of the molecule through the nitrogen atom.

“Arylheteroalkyl” means an aryl-heteroalkyl-group in which the aryl and heteroalkyl moieties are as defined herein. The group may be a terminal group or a bridging group. If the group is a terminal group it is bonded to the remainder of the molecule through the heteroalkyl group.

“Aryloxy” refers to an aryl-O— group in which the aryl is as defined herein. Preferably the aryloxy is a C₆-C₁₈aryloxy, more preferably a C₆-C₁₀ aryloxy. The group may be a terminal group or a bridging group. If the group is a terminal group it is bonded to the remainder of the molecule through the oxygen atom.

“Arylsulfonyl” means an aryl-S(═O)₂— group in which the aryl group is as defined herein. The group may be a terminal group or a bridging group. If the group is a terminal group it is bonded to the remainder of the molecule through the sulfur atom.

A “bond” is a linkage between atoms in a compound or molecule. The bond may be a single bond, a double bond, or a triple bond.

“Cycloalkenyl” means a non-aromatic monocyclic or multicyclic ring system containing at least one carbon-carbon double bond and preferably having from 5-10 carbon atoms per ring. Exemplary monocyclic cycloalkenyl rings include cyclopentenyl, cyclohexenyl or cycloheptenyl. The cycloalkenyl group may be substituted by one or more substituent groups. A cycloalkenyl group typically is a C₃-C₁₂ alkenyl group. The group may be a terminal group or a bridging group.

“Cycloalkyl” refers to a saturated monocyclic or fused or spiro polycyclic, carbocycle preferably containing from 3 to 9 carbons per ring, such as cyclopropyl, cyclobutyl, cyclopentyl, cyclohexyl and the like, unless otherwise specified. It includes monocyclic systems such as cyclopropyl and cyclohexyl, bicyclic systems such as decalin, and polycyclic systems such as adamantane. A cycloalkyl group typically is a C₃-C₁₂ alkyl group. The group may be a terminal group or a bridging group.

“Cycloalkylalkyl” means a cycloalkyl-alkyl-group in which the cycloalkyl and alkyl moieties are as defined herein. Exemplary monocycloalkylalkyl groups include cyclopropylmethyl, cyclopentylmethyl, cyclohexylmethyl and cycloheptylmethyl. The group may be a terminal group or a bridging group. If the group is a terminal group it is bonded to the remainder of the molecule through the alkyl group.

“Cycloalkylalkenyl” means a cycloalkyl-alkenyl-group in which the cycloalkyl and alkenyl moieties are as defined herein. The group may be a terminal group or a bridging group. If the group is a terminal group it is bonded to the remainder of the molecule through the alkenyl group.

“Cycloalkylheteroalkyl” means a cycloalkyl-heteroalkyl-group in which the cycloalkyl and heteroalkyl moieties are as defined herein. The group may be a terminal group or a bridging group. If the group is a terminal group it is bonded to the remainder of the molecule through the heteroalkyl group.

“Cycloalkyloxy” refers to a cycloalkyl-O— group in which cycloalkyl is as defined herein. Preferably the cycloalkyloxy is a C₁-C₆cycloalkyloxy. Examples include, but are not limited to, cyclopropanoxy and cyclobutanoxy. The group may be a terminal group or a bridging group. If the group is a terminal group it is bonded to the remainder of the molecule through the oxygen atom.

“Cycloalkenyloxy” refers to a cycloalkenyl-O— group in which the cycloalkenyl is as defined herein. Preferably the cycloalkenyloxy is a C₁-C₆cycloalkenyloxy. The group may be a terminal group or a bridging group. If the group is a terminal group it is bonded to the remainder of the molecule through the oxygen atom.

“Cycloamino” refers to a saturated monocyclic, bicyclic, or polycyclic ring containing at least one nitrogen in at least one ring. Each ring is preferably from 3 to 10 membered, more preferably 4 to 7 membered. The group may be a terminal group or a bridging group. If the group is a terminal group it is bonded to the remainder of the molecule through the nitrogen atom.

“Haloalkyl” refers to an alkyl group as defined herein in which one or more of the hydrogen atoms has been replaced with a halogen atom selected from the group consisting of fluorine, chlorine, bromine and iodine. A haloalkyl group typically has the formula CₙH₍₂ₙ₊₁₋ₘ₎Xₘ wherein each X is independently selected from the group consisting of F, Cl, Br and I. In groups of this type n is typically from 1 to 10, more preferably from 1 to 6, most preferably 1 to 3. m is typically 1 to 6, more preferably 1 to 3. Examples of haloalkyl include fluoromethyl, difluoromethyl and trifluoromethyl.

“Haloalkenyl” refers to an alkenyl group as defined herein in which one or more of the hydrogen atoms has been replaced with a halogen atom independently selected from the group consisting of F, Cl, Br and I.

“Haloalkynyl” refers to an alkynyl group as defined herein in which one or more of the hydrogen atoms has been replaced with a halogen atom independently selected from the group consisting of F, Cl, Br and I.

“Halogen” represents chlorine, fluorine, bromine or iodine.

“Heteroalkyl” refers to a straight- or branched-chain alkyl group preferably having from 2 to 12 carbons, more preferably 2 to 6 carbons in the chain, one or more of which has been replaced by a heteroatom selected from S, O, P and N. Exemplary heteroalkyls include alkyl ethers, secondary and tertiary alkyl amines, amides, alkyl sulfides, and the like. Examples of heteroalkyl also include hydroxyC₁-C₅alkyl, C₁-C₆alkyloxyC₁-C₆alkyl, aminoC₁-C₆alkyl, C₁-C₆alkylaminoC₁-C₆alkyl, and di(C₁-C₆alkyl)aminoC₁-C₆alkyl. The group may be a terminal group or a bridging group.

“Heteroalkyloxy” refers to an heteroalkyl-O— group in which heteroalkyl is as defined herein. Preferably the heteroalkyloxy is a C₁-C₆heteroalkyloxy. The group may be a terminal group or a bridging group.

“Heteroaryl” either alone or part of a group refers to groups containing an aromatic ring (preferably a 5 or 6 membered aromatic ring) having one or more heteroatoms as ring atoms in the aromatic ring with the remainder of the ring atoms being carbon atoms. Suitable heteroatoms include nitrogen, oxygen and sulphur. Examples of heteroaryl include thiophene, benzothiophene, benzofuran, benzimidazole, benzoxazole, benzothiazole, benzisothiazole, naphtho[2,3-b]thiophene, furan, isoindolizine, xantholene, phenoxatine, pyrrole, imidazole, pyrazole, pyridine, pyrazine, pyrimidine, pyridazine, tetrazole, indole, isoindole, 1H-indazole, purine, quinoline, isoquinoline, phthalazine, naphthyridine, quinoxaline, cinnoline, carbazole, phenanthridine, acridine, phenazine, thiazole, isothiazole, phenothiazine, oxazole, isooxazole, furazane, phenoxazine, 2-, 3- or 4-pyridyl, 2-, 3-, 4-, 5-, or 8-quinolyl, 1-, 3-, 4-, or 5-isoquinolinyl 1-, 2-, or 3-indolyl, and 2-, or 3-thienyl. A heteroaryl group is typically a C₁-C₁₈ heteroaryl group. The group may be a terminal group or a bridging group. Typically a heteroaryl group is a 3 to 18 membered group.

“Heteroarylalkyl” means a heteroaryl-alkyl group in which the heteroaryl and alkyl moieties are as defined herein. Preferred heteroarylalkyl groups contain a lower alkyl moiety. Exemplary heteroarylalkyl groups include pyridylmethyl. The group may be a terminal group or a bridging group. If the group is a terminal group it is bonded to the remainder of the molecule through the alkyl group.

“Heteroarylalkenyl” means a heteroaryl-alkenyl-group in which the heteroaryl and alkenyl moieties are as defined herein. The group may be a terminal group or a bridging group. If the group is a terminal group it is bonded to the remainder of the molecule through the alkenyl group.

“Heteroarylheteroalkyl” means a heteroaryl-heteroalkyl-group in which the heteroaryl and heteroalkyl moieties are as defined herein. The group may be a terminal group or a bridging group. If the group is a terminal group it is bonded to the remainder of the molecule through the heteroalkyl group.

“Heteroarylamino” refers to groups containing an aromatic ring (preferably 5 or 6 membered aromatic ring) having at least one nitrogen and at least another heteroatom as ring atoms in the aromatic ring, preferably from 1 to 3 heteroatoms in at least one ring. Suitable heteroatoms include nitrogen, oxygen and sulphur. Arylamino and aryl is as defined herein. The group may be a terminal group or a bridging group. If the group is a terminal group it is bonded to the remainder of the molecule through the nitrogen atom.

“Heteroaryloxy” refers to a heteroaryl-O— group in which the heteroaryl is as defined herein. Preferably the heteroaryloxy is a C₁-C₁₈heteroaryloxy. The group may be a terminal group or a bridging group. If the group is a terminal group it is bonded to the remainder of the molecule through the oxygen atom.

“Heterocyclic” refers to saturated, partially unsaturated or fully unsaturated monocyclic, bicyclic or polycyclic ring system containing at least one heteroatom selected from the group consisting of nitrogen, sulfur and oxygen as a ring atom. Examples of heterocyclic moieties include heterocycloalkyl, heterocycloalkenyl and heteroaryl.

“Heterocycloalkenyl” refers to a heterocycloalkyl as defined herein but containing at least one double bond. A heterocycloalkenyl group typically is a C₂-C₁₂ heterocycloalkenyl group. The group may be a terminal group or a bridging group.

“Heterocycloalkyl” refers to a saturated monocyclic, bicyclic, or polycyclic ring containing at least one heteroatom selected from nitrogen, sulfur, oxygen, preferably from 1 to 3 heteroatoms in at least one ring. Each ring is preferably from 3 to 10 membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane, and 1,4-oxathiapane. A heterocycloalkyl group typically is a C₂-C₁₂ heterocycloalkyl group. The group may be a terminal group or a bridging group.

“Heterocycloalkylalkyl” refers to a heterocycloalkyl-alkyl-group in which the heterocycloalkyl and alkyl moieties are as defined herein. Exemplary heterocycloalkylalkyl groups include (2-tetrahydrofuryl)methyl, (2-tetrahydrothiofuranyl) methyl. The group may be a terminal group or a bridging group. If the group is a terminal group it is bonded to the remainder of the molecule through the alkyl group.

“Heterocycloalkylalkenyl” refers to a heterocycloalkyl-alkenyl-group in which the heterocycloalkyl and alkenyl moieties are as defined herein. The group may be a terminal group or a bridging group. If the group is a terminal group it is bonded to the remainder of the molecule through the alkenyl group.

“Heterocycloalkylheteroalkyl” means a heterocycloalkyl-heteroalkyl-group in which the heterocycloalkyl and heteroalkyl moieties are as defined herein. The group may be a terminal group or a bridging group. If the group is a terminal group it is bonded to the remainder of the molecule through the heteroalkyl group.

“Heterocycloalkyloxy” refers to a heterocycloalkyl-O— group in which the heterocycloalkyl is as defined herein. Preferably the heterocycloalkyloxy is a C₁-C₆heterocycloalkyloxy. The group may be a terminal group or a bridging group. If the group is a terminal group it is bonded to the remainder of the molecule through the oxygen atom.

“Heterocycloalkenyloxy” refers to a heterocycloalkenyl-O— group in which heterocycloalkenyl is as defined herein. Preferably the Heterocycloalkenyloxy is a C₁-C₆ Heterocycloalkenyloxy. The group may be a terminal group or a bridging group. If the group is a terminal group it is bonded to the remainder of the molecule through the oxygen atom.

“Heterocycloamino” refers to a saturated monocyclic, bicyclic, or polycyclic ring containing at least one nitrogen and at least another heteroatom selected from nitrogen, sulfur, oxygen, preferably from 1 to 3 heteroatoms in at least one ring. Each ring is preferably from 3 to 10 membered, more preferably 4 to 7 membered. The group may be a terminal group or a bridging group. If the group is a terminal group it is bonded to the remainder of the molecule through the nitrogen atom.

“Hydroxyalkyl” refers to an alkyl group as defined herein in which one or more of the hydrogen atoms has been replaced with an OH group. A hydroxyalkyl group typically has the formula CₙH₍₂ₙ₊₁₋ₓ₎(OH)ₓ. In groups of this type n is typically from 1 to 10, more preferably from 1 to 6, most preferably from 1 to 3. x is typically from 1 to 6, more preferably from 1 to 4.

“Lower alkyl” as a group means unless otherwise specified, an aliphatic hydrocarbon group which may be straight or branched having 1 to 6 carbon atoms in the chain, more preferably 1 to 4 carbons such as methyl, ethyl, propyl (n-propyl or isopropyl) or butyl (n-butyl, isobutyl or tertiary-butyl). The group may be a terminal group or a bridging group.

“Subject” refers to a human or an animal.

“Sulfinyl” means an R—S(═O)— group in which the R group may be OH, alkyl, cycloalkyl, heterocycloalkyl; aryl or heteroaryl group as defined herein. The group may be a terminal group or a bridging group. If the group is a terminal group it is bonded to the remainder of the molecule through the sulfur atom.

“Sulfinylamino” means an R—S(═O)—NH— group in which the R group may be OH, alkyl, cycloalkyl, heterocycloalkyl; aryl or heteroaryl group as defined herein. The group may be a terminal group or a bridging group. If the group is a terminal group it is bonded to the remainder of the molecule through the nitrogen atom.

“Sulfonyl” means an R—S(═O)₂— group in which the R group may be OH, alkyl, cycloalkyl, heterocycloalkyl; aryl or heteroaryl group as defined herein. The group may be a terminal group or a bridging group. If the group is a terminal group it is bonded to the remainder of the molecule through the sulfur atom.

“Sulfonylamino” means an R—S(═O)₂—NH— group. The group may be a terminal group or a bridging group. If the group is a terminal group it is bonded to the remainder of the molecule through the nitrogen atom.

It is understood that included in the family of compounds of Formula (I) are isomeric forms including diastereoisomers, enantiomers, tautomers, and geometrical isomers in “E” or “Z” configurational isomer or a mixture of E and Z isomers. It is also understood that some isomeric forms such as diastereomers, enantiomers, and geometrical isomers can be separated by physical and/or chemical methods and by those skilled in the art.

Some of the compounds of the disclosed embodiments may exist as single stereoisomers, racemates, and/or mixtures of enantiomers and/or diastereomers. All such single stereoisomers, racemates and mixtures thereof, are intended to be within the scope of the subject matter described and claimed.

Additionally, Formula (I) is intended to cover, where applicable, solvated as well as unsolvated forms of the compounds. Thus, each formula includes compounds having the indicated structure, including the hydrated as well as the non-hydrated forms.

Further, it is possible that compounds of the invention may contain more than one asymmetric carbon atom. In those compounds, the use of a solid line to depict bonds to asymmetric carbon atoms is meant to indicate that all possible stereoisomers are meant to be included. The use of a solid line to depict bonds to one or more asymmetric carbon atoms in a compound of the invention and the use of a solid or dotted wedge to depict bonds to other asymmetric carbon atoms in the same compound is meant to indicate that a mixture of diastereomers is present.

The term “optionally substituted” as used herein means the group to which this term refers may be unsubstituted, or may be substituted with one or more groups independently selected from alkyl, alkenyl, alkynyl, thioalkyl, cycloalkyl, cycloalkylalkyl, cycloalkenyl, cycloalkylalkenyl, heterocycloalkyl, cycloalkylheteroalkyl, cycloalkyloxy, cycloalkenyloxy, cycloamino, halo, carboxyl, haloalkyl, haloalkynyl, alkynyloxy, heteroalkyl, heteroalkyloxy, hydroxyl, hydroxyalkyl, alkoxy, thioalkoxy, alkenyloxy, haloalkoxy, haloalkenyl, haloalkynyl, haloalkenyloxy, nitro, amino, nitroalkyl, nitroalkenyl, nitroalkynyl, nitroheterocyclyl, alkylamino, dialkylamino, alkenylamine, aminoalkyl, alkynylamino, acyl, alkyloxy, alkyloxyalkyl, alkyloxyaryl, alkyloxycarbonyl, alkyloxycycloalkyl, alkyloxyheteroaryl, alkyloxyheterocycloalkyl, alkenoyl, alkynoyl, acylamino, diacylamino, acyloxy, alkylsulfonyloxy, heterocyclic, heterocycloalkenyl, heterocycloalkyl, heterocycloalkylalkyl, heterocycloalkylalkenyl, heterocycloalkylheteroalkyl, heterocycloalkyloxy, heterocycloalkenyloxy, heterocycloxy, heterocycloamino, haloheterocycloalkyl, alkylsulfinyl, alkylsulfonyl, alkylsulfenyl, alkylcarbonyloxy, alkylthio, acylthio, aminosulfonyl, phosphorus-containing groups such as phosphono and phosphinyl, sulfinyl, sulfinylamino, sulfonyl, sulfonylamino, aryl, heteroaryl, heteroarylalkyl, heteroarylalkenyl, heteroarylheteroalkyl, heteroarylamino, heteroaryloxy, arylalkenyl, arylalkyl, alkylaryl, alkylheteroaryl, aryloxy, arylsulfonyl, cyano, cyanate, isocyanate, —C(O)NH(alkyl), and —C(O)N(alkyl)₂.

The term “optionally substituted alkyl”, for the purpose of this this application, refers to an optionally substituted linear alkyl or an optionally substituted branched alkyl. The optionally substituted linear alkyl may be an optionally substituted C₁ to C₂₅ linear alkyl. The optionally substituted branched alkyl may be an optionally substituted C₁ to C₂₅ branched alkyl. The optionally substituted branched alkyl may have the following formula (A), where h may be 0 or 1. i and j may independently be integers selected from 1 to 13. h may be 1. i may be 7. j may be 9.

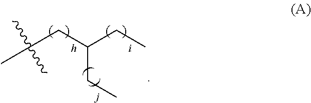

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 is a representative scheme for the synthesis of the polymer 1, polymer 2 and polymer 3.

FIG. 2 shows the GPC curve and the reading of polymer 1.

FIG. 3 shows the GPC curve and the reading of polymer 2.

FIG. 4 shows the GPC curve and the reading of polymer 3.

FIG. 5 are UV-visible spectra of solutions of the polymer 1, polymer 2 and polymer 3 in chlorobenzene and thin films.

FIG. 6 is a schematic drawing of the polymer device.

FIG. 7 is a UV-vis-NIR spectrum of a polymer 1 device at various applied potentials (V). (a) 0.0, (b) 0.2, (c) 0.4, (d) 0.5, (e) 0.6, (f) 0.7, (g) 0.8, (h) 0.9, (i) 1.0, (j) 1.1, (k) 1.2, (l) 1.3, (m) 1.4, (n) 1.5, (o) 1.6, (p) 1.7, (q) 1.8 V.

FIG. 8 is a UV-vis-NIR spectrum of a polymer 2 device at various applied potentials (V). (a) 0.0, (b) 0.2, (c) 0.4, (d) 0.5, (e) 0.6, (f) 0.7, (g) 0.8, (h) 0.9, (i) 1.0, (j) 1.1, (k) 1.2, (l) 1.3, (m) 1.4, (n) 1.5, (o) 1.6, (p) 1.7, (q) 1.8 V.

FIG. 9 is a UV-vis-NIR spectrum of a polymer 3 device at various applied potentials (V). (a) 0.0, (b) 0.2, (c) 0.4, (d) 0.5, (e) 0.6, (f) 0.7, (g) 0.8, (h) 0.9, (i) 1.0, (j) 1.1, (k) 1.2, (l) 1.3, (m) 1.4, (n) 1.5, (o) 1.6, (p) 1.7, (q) 1.8 V.

FIG. 10 is a square-wave potential step absorptiometry spectrum of a polymer 1 device between 1.5 V and −1.5 V with a switch time of 10 seconds.

FIG. 11 is a square-wave potential step absorptiometry spectrum of a polymer 2 device between 1.5 V and −1.5 V with a switch time of 10 seconds.

FIG. 12 is a square-wave potential step absorptiometry spectrum of a polymer 3 device between 1.5 V and −1.5 V with a switch time of 10 seconds.

FIG. 13 are photos of polymers 1, polymer 2 and polymer 3 (from left to right) devices in their neutral (top) and 2.0 V (bottom) states.

FIG. 14 is a representative scheme for the synthesis of the polymer 4, polymer 5 and polymer 6.

FIG. 15 is a ¹H-NMR spectrum of a synthesized compound, recorded in CDCl₃.

FIG. 16 is a ¹³C-NMR spectrum of a synthesized intermediate, recorded in CDCl₃.

FIG. 17 is a HRMS spectrum of a synthesized intermediate.

FIG. 18 is a ¹H-NMR spectrum of the synthesized polymer 4, recorded in CDCl₃.

FIG. 19 is a ¹H-NMR spectrum of the synthesized polymer 5, recorded in CDCl₃.

FIG. 20 is a ¹H-NMR spectrum of the synthesized polymer 6, recorded in CDCl₃.

FIG. 21 is a EC electrospectroscopy of polymer 5 in a EC device.

FIG. 22 is a Time-dependent density-functional theory calculation, carried out to compare the magnitude of electron deficiency of the imide-fused pyridazines with current commonly used electron deficient acceptors.

FIG. 23 is a HR-EI MS spectrum of a synthesized intermediate.

### DETAILED DESCRIPTION OF EMBODIMENTS

A compound may have the following Formula (I);

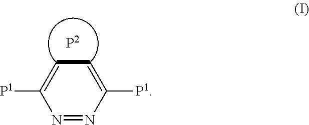

A compound may have repeating units of the following Formula (IV);

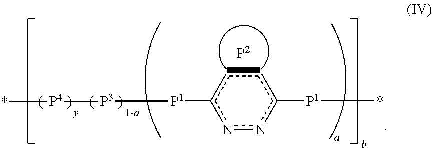

A compound may have repeating units of the following formula (IVa) or (IVb):

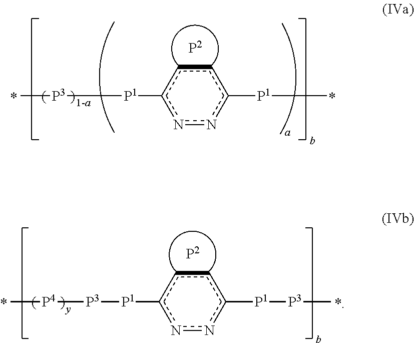

A compound may have the following Formula (VI);

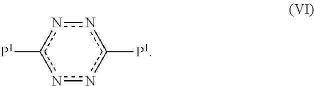

A compound may have the following formula (VIIa) or (VIIb);

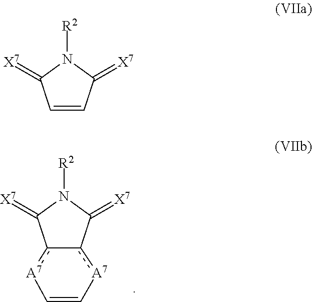

P¹ may have the following Formula (IIa) or (IIb);

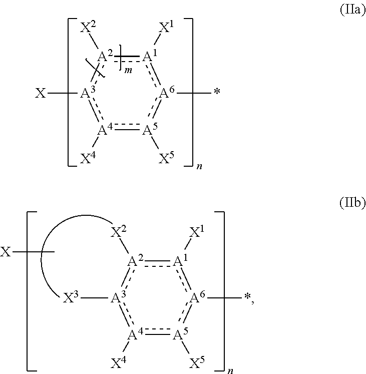

P² may have the following formula (IIIa) or (IIIb);

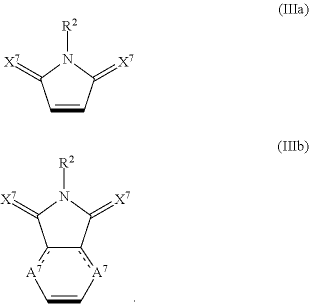

P³ has the following Formula (Va), (Vb) or (Vc);

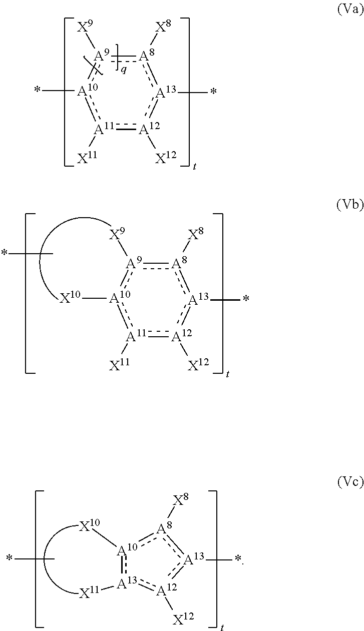

P⁴ may be absent, or an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl.

[symbol] may be a single or double bond, as valency allows.

m may be 0 or 1.

n may be an integer selected from 1 to 4.

X, X¹, X², X³, X⁴ and X⁵ may independently be absent or selected from the group consisting of a bond, H, halogen, CN, R¹, OR¹ or SR¹.

Halogen may be selected from the group consisting of F, Cl, Br and I.

X may be selected from the group consisting of halogen, CN, R¹ and OR¹.

R¹ may be selected from the group consisting of optionally substituted alkyl, optionally substituted alkenyl, optionally substituted alkynyl, optionally substituted cycloalkyl, optionally substituted heterocycloalkyl, optionally substituted cycloalkenyl, optionally substituted heterocycloalkenyl, optionally substituted aryl, optionally substituted heteroaryl. R¹ may be an optionally substituted alkyl.

A¹ may be selected from the group consisting of O, S, Se, Te, C and N.

A², A³, A⁴, A⁵ and A⁶ may independently be C or N.

Any two substituents selected from the group consisting of X¹, X², X³, X⁴ and X⁵ may optionally be taken together to form an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl.

R² may be an optionally substituted alkyl or optionally substituted aryl.

X⁷ may be O or S.

A⁷ may be CH, CH₂ or N.

[symbol] may be where the compound of Formula (IIIa) or (IIIb) is fused to the compound of Formula (I) of Formula (IV).

q may be 0 or 1.

t may be an integer selected from 1 to 4.

X⁸, X⁹, X¹¹ and X¹² may independently be absent or selected from the group consisting of a bond, H, halogen, CN, R³, OR³ or SR³.

R³ may be selected from the group consisting of optionally substituted alkyl, optionally substituted alkenyl, optionally substituted alkynyl, optionally substituted cycloalkyl, optionally substituted heterocycloalkyl, optionally substituted cycloalkenyl, optionally substituted heterocycloalkenyl, optionally substituted aryl, optionally substituted heteroaryl.

A⁸ may be selected from the group consisting of O, S, Se, Te, C and N.

A⁹, A¹⁰, A¹¹, A¹² and A¹³ may independently be C or N.

Any two substituents selected from the group consisting of X⁸, X⁹, X¹⁰, X¹¹ and X¹² may optionally be taken together to form an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl.

a may be any value from 0 to 1.

b may be an integer selected from 1 to 10,000.

y may be an integer selected from 0 to 5.

Optionally substituted alkyl may be an optionally substituted C₁-C₂₅ alkyl, optionally substituted alkenyl may be an optionally substituted C₂-C₂s alkenyl, optionally substituted alkynyl may be an optionally substituted C₂-C₂₅ alkynyl, optionally substituted cycloalkyl may be an optionally substituted C₃-C₁₂ cycloalkyl, optionally substituted heterocycloalkyl may be an optionally substituted C₃-C₁₂ heterocycloalkyl, optionally substituted cycloalkenyl may be an optionally substituted C₃-C₁₂ cycloalkenyl, optionally substituted heterocycloalkenyl may be an optionally substituted C₃-C₁₂ heterocycloalkenyl, optionally substituted aryl may be an optionally substituted C₃-C₁₈ aryl and optionally substituted heteroaryl may be an optionally substituted C₃-C₁₈ heteroaryl.

Optionally substituted heterocycloalkyl and optionally substituted heteroaryl may independently be a 3- to 18-membered monocyclic, bicyclic, or polycyclic ring.

P¹

P¹ may be selected from the group consisting of optionally substituted furan, optionally substituted thiophene, optionally substituted selenophene, optionally substituted tellurophene, optionally substituted pyrrole, optionally substituted phenylene, optionally substituted aza-phenylene, optionally substituted arylene diimide, optionally substituted fluorene, optionally substituted cabazole, optionally substituted dibenzodilole, optionally substituted benzooxodiazole, optionally substituted benzotriazole, optionally substituted benzothidiazole and any combination thereof.

The P¹ may have the following Formula (IIa);

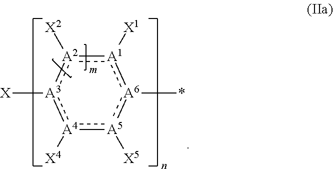

m may be 0.

A¹ may be N. A¹ may be S.

A³, A⁴, A⁵ and A⁶ may be C.

X¹ may be H. X¹ may be absent.

X⁴ and X⁵ may independently be selected from the group consisting of H, R¹ and OR¹. R¹ may be an optionally substituted alkyl. X⁴ and X⁵ may be H.

X may be Br.

When m is 0, A¹ may be N, and X¹ may be H or optionally substituted alkyl.

When m is 0, A³, A⁴, A⁵ and A⁶ may be C, and X⁴ and X⁵ are independently selected from the group consisting of H, R¹ and OR¹, wherein R¹ is optionally substituted alkyl.

When m is 0, A¹ may be S; X¹ may be absent; A³, A⁴, A⁵ and A⁶ may be C, X⁴ and X⁵ may be H and X may be Br,

When m is 0, A¹ may be O; X¹ may be absent; A³, A⁴, A⁵ and A⁶ may be C, X⁴ and X⁵ may be H and X may be Br.

The compound may have the following formula (VIIIa) or (VIIIb):

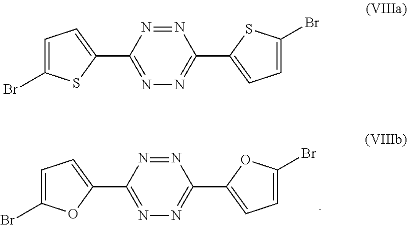 [SMILES: Brc1ccc(-c2nnc(-c3ccc(Br)s3)nn2)s1 ; Brc1ccc(-c2nnc(-c3ccc(Br)o3)nn2)o1]

m may be 1.

A¹, A² A³, A⁴, A⁵ and A⁶ may be C. A¹, A² A³ and A⁶ may be C. A³ and A⁶ may be C. A⁴ and A⁵ may be N. A⁴ and A⁵ may be C.

X¹, X², X⁴ and X⁶ may independently be absent or selected from the group consisting of H, halogen, R¹ and OR¹. X¹, X², X⁴ and X⁶ may independently be H or halogen. X⁴ and X⁵ may independently be H or F.

R¹ may be an optionally substituted alkyl.

X¹and X² may be taken together to form an optionally substituted 5-membered heteroaryl.

When m is 1, A³ and A⁶ may be C and X¹, X², X⁴ and X⁶ may be independently absent or selected from the group consisting of H, halogen, R¹ and OR¹, wherein R¹ is optionally substituted alkyl.

When m is 1, A¹, A² A³, A⁴, A⁵ and A⁶ may be C and X¹, X², X⁴ and X⁶ may independently be H or halogen.

When m is 1, A¹, A² A³, A⁴, A⁵ and A⁶ may be C and X¹, X², X⁴ and X⁶ may be H.

The compound may have the following Formula (VIIIc):

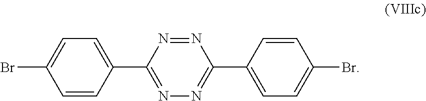 [SMILES: Brc1ccc(-c2nnc(-c3ccc(Br)cc3)nn2)cc1]

When m is 1, A¹, A² A³ and A⁶ may be C and X¹ and X² may be taken together to form an optionally substituted 5-membered heteroaryl.

P¹ may have the following formula (IX);

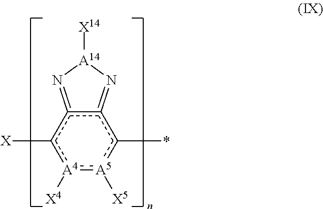

A¹⁴ may be selected from the group consisting of O, S, Se, Te and N.

X¹⁴ may be absent or optionally substituted alkyl.

When P¹ has the formula (IX), A⁴ and A⁵ may be N.

When P1 has the formula (IX), A⁴ and A⁵ may be C; and X⁴ and X⁵ may independently be H or F.

P¹ may have the following Formula (IIb);

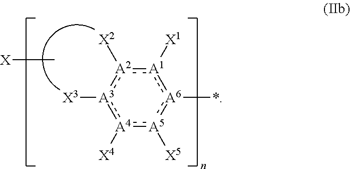

A¹, A² A³, A⁴, A⁵ and A⁶ may be C.

X¹, X⁴ and X⁵ may be H.

X² and X³ may be taken together to form an optionally substituted 5-membered heterocycloalkyl. X² and X³ may be taken together to form an optionally substituted 6-membered heterocycloalkyl. X³ and X⁴ may be taken together to form an optionally substituted a 6-membered aryl. X⁴ and X⁵ may be taken together to form an optionally substituted 6-membered heterocycloalkyl.

The optionally substituted 5-membered heterocycloalkyl may be further fused with an optionally substituted 6-membered aryl.

P¹ may have the following formula (X);

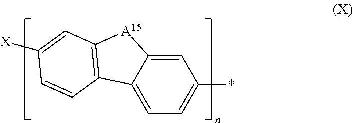

A¹⁵ may be selected from the group consisting of NH, C(R⁴)₂, and Si(R⁴)₂. R⁴ may be an optionally substituted alkyl.

P¹ may have the following Formula (XI);

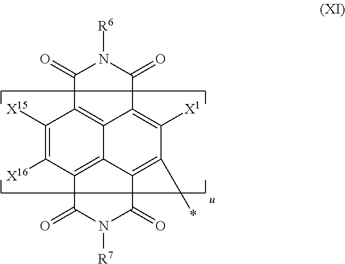

X¹, X¹⁵ and X¹⁶ may independently be selected from the group consisting of H, halogen and CN.

R⁶ and R⁷ may independently be selected from the group consisting of H, R⁸ and OR⁸, wherein R⁸ is optionally substituted alkyl.

u may be 1 or 2.

P²

P² may be selected from the group consisting of optionally substituted imide and optionally substituted phthalamide.

R² may be an optionally substituted alkyl. The optionally substituted alkyl may be an optionally substituted linear alkyl or an optionally substituted branched alkyl. The optionally substituted linear alkyl may be an optionally substituted C₁ to C₂₅ linear alkyl. The optionally substituted branched alkyl may be an optionally substituted C₁ to C₂₅ branched alkyl. The optionally substituted branched alkyl may have the following formula (XII).

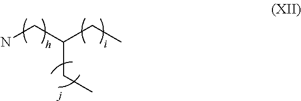

N of Formula (XII) may be where the R² group is connected to the rest of the compound in Formulae (IVa), (IVb), (IVc), (IVd), (VIIa) or (VIIb).

h may be 0 or 1. i and j may independently be integers selected from 1 to 13. h may be 1. i may be 7. j may be 9.

The optionally substituted alkyl may be 2-ethylheptyl, 2-heptylhexadecyl, 2-heptylpentadecyl, 2-heptyltetradecyl, 2-heptyltridecyl, 2-heptyldodeclyl, 2-heptylundecyl, 2-heptyldecyl, 2-heptylnonyl, 2-heptyloctyl, 2-heptylheptyl, 2-octylhexadecyl, 2-octylpentadecyl, 2-octyltetradecyl, 2-octyltridecyl, 2-octyldodecyl, 2-octylundecyl, 2-octyldecyl, 2-octylnonyl, 2-octyloctyl, 2-nonyltetradecyl, 2-nonyltridecyl, 2-nonyldodecyl, 2-nonylundecyl, 2-nonyldecyl or 2-nonylnonyl.

The optionally substituted may be any linear or branched alkyl chain that may confer improved solubility and processability of the final product.

X7 may be O.

R2 may be an optionally substituted aryl having the following Formula (XIII);

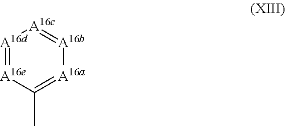

A^{16a}, A^{16b}, A^{16c}, A^{16d} and A^{16e} may independently be selected from the group consisting of N, CH and CR⁹, wherein R⁹ is an optionally substituted alkyl. A^{16a} may be N and A^{16b}, A^{16c}, A^{16d} and A^{16e} may be CH. A^{16b} may be N and A^{16a}, A^{16c}, A^{16d}, A^{16e} may be CH. A^{16c} may be N and A^{16a}, A^{16b}, A^{16d} and A^{16e} may be CH. A^{16b} and A^{16d} may be N and A^{16a}, A^{16c}, A^{16d} and A^{16e} may be CH. A^{16a}, A^{16c}, A^{16e} may be N and A^{16b} and A^{16d} may be CH.

A⁷ may be CH. A⁷ may be CH₂.

P² may have the following formula (IIIa), (IIIb), (IIIc), or (IIId):

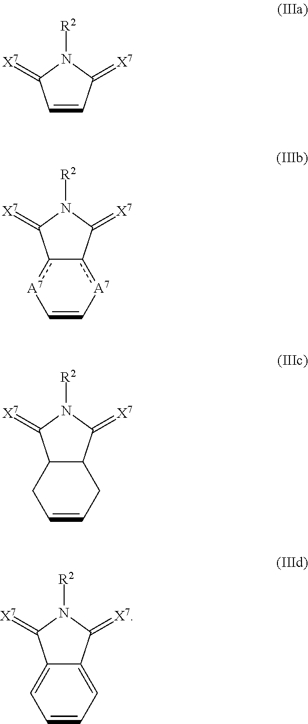

The compound may have the following formula (XIV), (XVa) or (XVb):

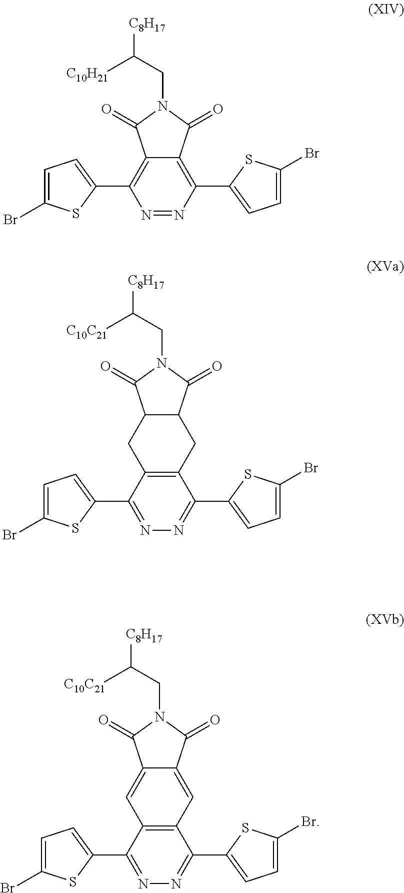

The compound of Formula (I) may be 6-alkyl-5H-pyrrolo[3,4-d]pyridazine-5,7(6H)-dione (PPD). PPD may lower the frontier orbitals of the polymers to make the polymer more stable under ambient conditions.

P³

P³ may be a moiety that performs as a good electron donor in electrochromic materials. The chemical and physical nature of the P³ group may enhance the solubility and processability of the final polymer materials as well as the performance of the polymer films under voltage bias.

P³ may be selected from the group consisting of optionally substituted furan, optionally substituted thiophene, optionally substituted selenophene, optionally substituted pyrrole, optionally substituted phenyl, optionally substituted fluorenes, optionally substituted carbazoles, optionally substituted fused furans and optionally substituted fused thiophenes.

P³ may have the following formula (Va);

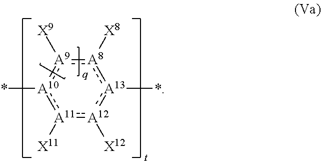

q may be 0.

A⁸ may be N. A⁸ may be S. A¹⁰, A¹¹, A¹² and A¹³ may be C.

X⁸ may be H or an optionally substituted alkyl. X⁸ may be absent. X¹¹ and X¹² may independently be selected from the group consisting of H, R³ and OR³. R³ may be optionally substituted alkyl.

X¹¹ and X¹² may be taken together to form an optionally substituted 7-membered heterocycloalkyl.

When q is 0, A⁸ may be N, A¹⁰, A¹¹, A¹² and A¹³ may be C, Xs may be H and X¹¹ and X¹² may independently be selected from the group consisting of H, R³ and OR3, wherein R³ is optionally substituted alkyl.

When q is 0, A⁸ may be S; X⁸may be absent; A¹⁰, A¹¹, A¹² and A¹³ may be C and X may be Br.

When q is 0, A⁸ may be S; X⁸ may be absent; A¹⁰, A¹¹, A¹² and A¹³ may be C, and X¹¹ and X¹² may be taken together to form an optionally substituted 7-membered heterocycloalkyl. The optionally substituted 7-membered heterocycloalkyl may be substituted with two alkoxy groups. The alkoxy group may be OC₁₂H₂₅.

q may be 1.

A⁸, A⁹ A¹⁰, A¹¹, A¹² and A¹³ may be C. A¹⁰ and A¹³ may be C.

X⁸, X⁹, X¹¹ and X¹² may independently be selected from the group consisting of H, halogen, R³ and OR³. R³ may be an optionally substituted alkyl. X⁸, X⁹, X¹¹ and X¹² may be independently selected from the group consisting of H, halogen, OR¹⁰ and SR¹⁰. R¹⁰ may be an optionally substituted alkyl.

When q is 1, A¹⁰ and A¹³ may be C; X⁸, X⁹, X¹¹ and X¹² may independently be selected from the group consisting of H, halogen, R³ and OR³, wherein R³ may be optionally substituted alkyl.

When q is 1, A⁸, A⁹ A¹⁰, A¹¹, A¹² and A¹³ may be C and X⁸, X⁹, X¹¹ and X¹² may independently be selected from the group consisting of H, halogen, OR¹⁰ and SR¹⁰, wherein R¹⁰ may be an optionally substituted alkyl.

P³ may be a 3,4-dialkoxythiophene.

P³ may have the following Formula (XVI);

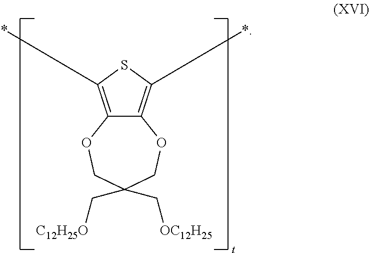

P³ may have the following formula (Vb);

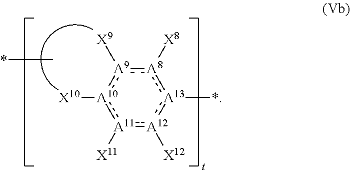

A⁸, A⁹ A¹⁰, A¹¹, A¹² and A¹³ may be C.

X⁸, X¹¹ and X¹² may be H.

X⁹ and X¹⁰ may be taken together to form an optionally substituted 5-membered heterocycloalkyl.

The optionally substituted 5-membered heterocycloalkyl may be further fused with an optionally substituted 6-membered aryl.

P³ may have the following formula (XVII);

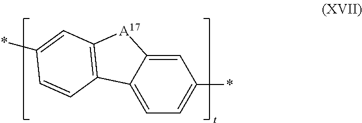

A¹⁷ may be selected from the group consisting of NH, C(R¹¹)₂, and Si(R¹¹)_{2.} R¹¹ may be optionally substituted alkyl. R¹¹ may be C₈H₁₇.

P³ may have the following Formula (Vc);

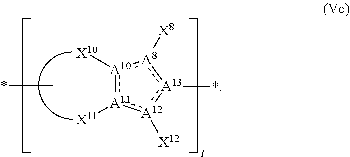

A¹⁰, A¹¹, A¹² and A¹³ may be C. X⁸ and X¹² may be H.

X¹⁰ and X¹¹ may be taken together to form an optionally substituted 5-membered heteroaryl. X¹⁰ and X¹¹ may be taken together to form an optionally substituted 6-membered aryl. The optionally substituted 6-membered aryl may be further fused with an optionally substituted 5-membered heteroaryl.

P³ may have the following Formula (XVIIIa) or (XVIIIb);

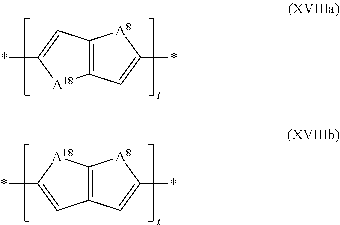

A⁸ and A¹⁸ may be independently selected from the group consisting of O, S and Se.

P³ may have the following Formula (XIX);

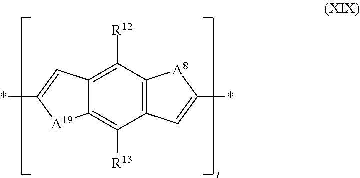

A⁸ and A¹⁹ may independently be selected from the group consisting of O, Se and Se. R¹² and R¹³ may independently be selected from the group consisting of R¹⁴, OR¹⁴ and SR¹⁴. R¹⁴ may be optionally substituted alkyl or optionally substituted aryl. R¹⁴ may be an optionally substituted alkyl. R¹⁴ may be C₈H_{17.} R¹⁴ may be a 2-ethylheptyl group.

P⁴

P⁴ may be absent, or selected from the group consisting of optionally substituted furan, optionally substituted thiophene, optionally substituted selenophene, optionally substituted pyrrole, optionally substituted phenyl, optionally substituted fluorenes, optionally substituted carbazoles, optionally substituted fused furans and optionally substituted fused thiophenes.

P⁴ may have the following formula (Va);

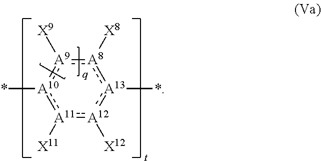

q may be 0.

A⁸ may be N. A⁸ may be S. A¹⁰, A¹¹, A¹² and A¹³ may be C.

X⁸ may be H or an optionally substituted alkyl. X⁸ may be absent. X¹¹ and X¹² may independently be selected from the group consisting of H, R³ and OR³. R³ may be optionally substituted alkyl.

X¹¹ and X¹² may be taken together to form an optionally substituted 7-membered heterocycloalkyl.

When q is 0, A⁸ may be N, A¹⁰, A¹¹, A¹² and A¹³ may be C, X8 may be H and X11 and X12 may independently be selected from the group consisting of H, R3 and OR3, wherein R3 is optionally substituted alkyl.

When q is 0, A⁸ may be S; X⁸ may be absent and A¹⁰, A¹¹, A¹² and A¹³ may be C.

When q is 0, A⁸ may be S; X⁸ may be absent; A¹⁰, A¹¹, A¹² and A¹³ may be C and X¹¹ and X¹² may be taken together to form an optionally substituted 7-membered heterocycloalkyl. The optionally substituted 7-membered heterocycloalkyl may be substituted with two alkoxy groups. The alkoxy groups may be OC₁₂H₂₅.

q may be 1.

A⁸, A⁹ A¹⁰, A¹¹, A¹² and A¹³ may be C. A¹⁰ and A¹³ may be C.

X⁸, X⁹, X¹¹ and X¹² may independently be selected from the group consisting of H, halogen, R³ and OR³. R³ may be an optionally substituted alkyl. X⁸, X⁹, X¹¹ and X¹² may be independently selected from the group consisting of H, halogen, OR¹⁰ and SR¹⁰. R¹⁰ may be an optionally substituted alkyl.

When q is 1, A¹⁰ and A¹³ may be C; X⁸, X⁹, X¹¹ and X¹² may independently be selected from the group consisting of H, halogen, R³ and OR³, wherein R³ may be optionally substituted alkyl.

When q is 1, A⁸, A⁹ A¹⁰, A¹¹, A¹² and A¹³ may be C and X⁸, X⁹, X¹¹ and X¹² may independently be selected from the group consisting of H, halogen, OR¹⁰ and SR¹⁰, wherein R¹⁰ may be an optionally substituted alkyl.

P⁴ may have the following formula (Vb);

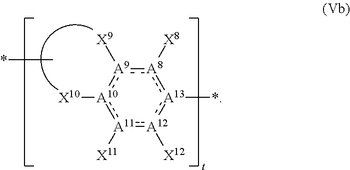

A⁸, A⁹ A¹⁰, A¹¹, A¹² and A¹³ may be C.

X⁸, X¹¹ and X¹² may be H.

X⁹ and X¹⁰ may be taken together to form an optionally substituted 5-membered heterocycloalkyl.

The optionally substituted 5-membered heterocycloalkyl may be further fused with an optionally substituted 6-membered aryl.

P⁴ may have the following formula (XVII);

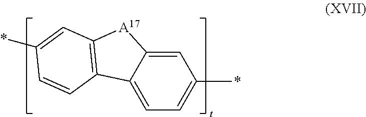

A¹⁷ may be selected from the group consisting of NH, CHR¹¹, C(R¹¹)₂, SiHR¹¹ and Si(R¹¹)_{2. R}¹¹ may be optionally substituted alkyl. R¹¹ may be C₈H₁₇ or C₁₂H₂₅.

P⁴ may have the following Formula (Vc);

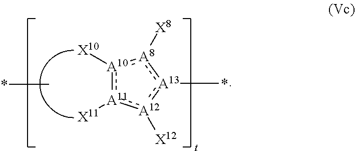

A¹⁰, A¹¹, A¹² and A¹³ may be C. X⁸ and X¹² may be H.

X¹⁰ and X¹¹ may be taken together to form an optionally substituted 5-membered heteroaryl. X¹⁰ and X¹¹ may be taken together to form an optionally substituted 6-membered aryl. The optionally substituted 6-membered aryl may be further fused with an optionally substituted 5-membered heteroaryl.

P⁴ may have the following Formula (XVIIIa) or (XVIIIb);

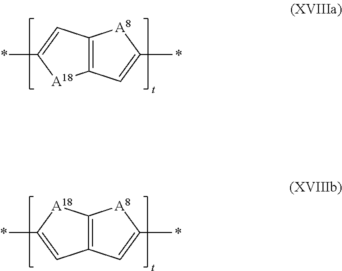

A⁸ and A¹⁸ may be independently selected from the group consisting of O, S and Se.

P⁴ may have the following Formula (XIX);

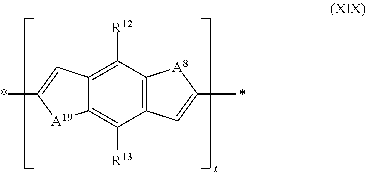

A⁸ and A¹⁹ may independently be selected from the group consisting of O, Se and Se. R¹² and R¹³ may independently be selected from the group consisting of R¹⁴, OR¹⁴ and SR¹⁴. R¹⁴ may be optionally substituted alkyl or optionally substituted aryl. R¹⁴ may be an optionally substituted alkyl.

P⁴ may be an optionally substituted thiophene or an optionally substituted fused thiophene.

P⁴ may have the following Formula (XXXIa) or (XXXIb):

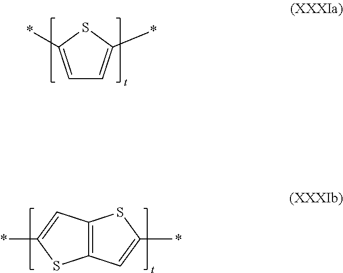

a may be 1, 0.9, 0.8, 0.7, 0.6, 0.5, 0.4, 0.3, 0.2 or 0.1. a may be 0.5, 0.3 or 0.1.

b may be an integer selected from 1 to 50, 1 to 10, 1 to 20, 1 to 30, 1 to 40, 10 to 20, 10 to 30, 10 to 40, 10 to 50, 20 to 30, 20 to 40, 20 to 50, 30 to 40, 30 to 50 or 40 to 50.

y may be 1. y may be 2.

The compound may have the following formula (XX):

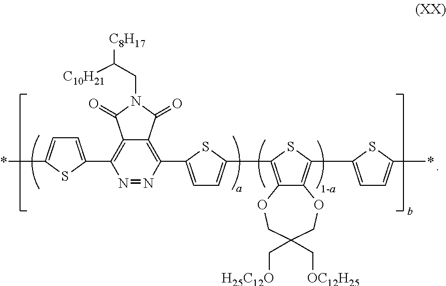

The compound may have the following formula (XXa), (XXb) or (XXc):

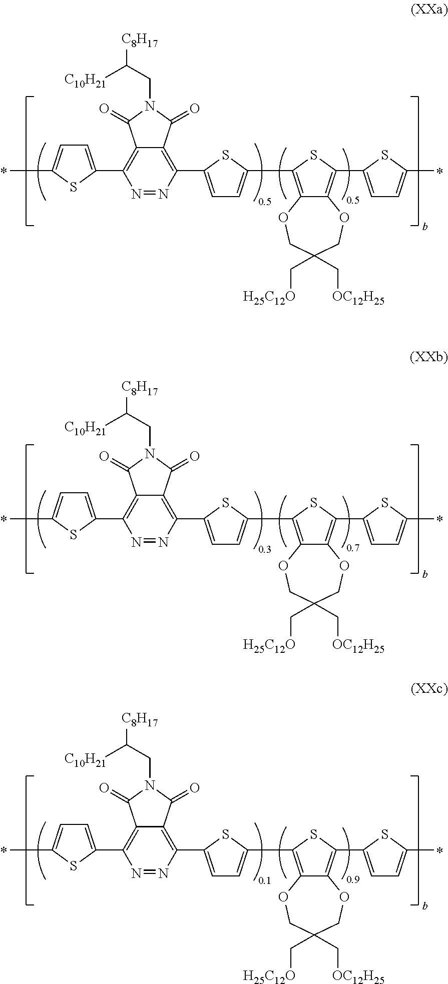

The compound may have the following formula (XXI):

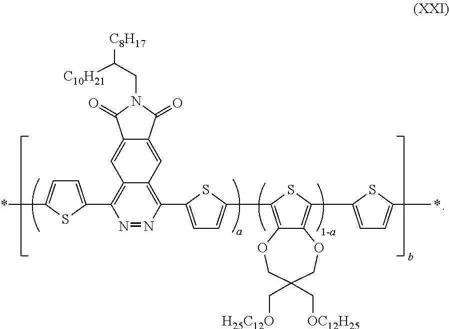

The compound may have the following formula (XXIa):

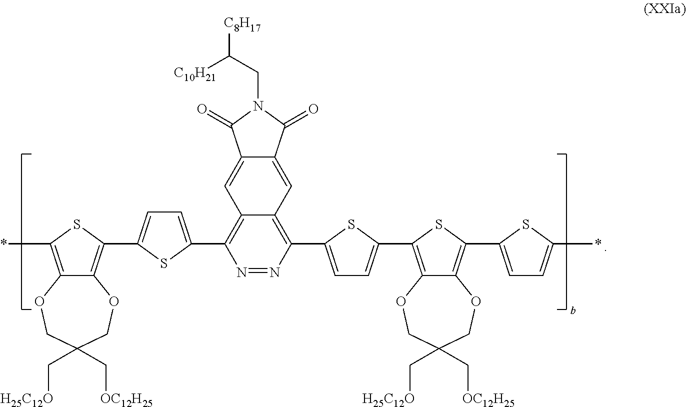

The compound may have the following formula (XXII):

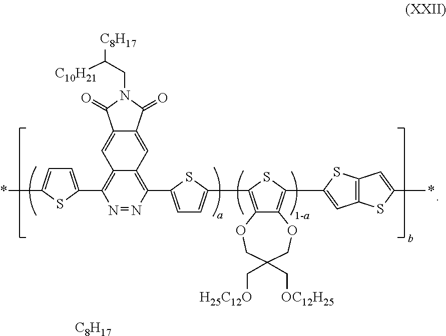

The compound may have the following formula (XXIIa):

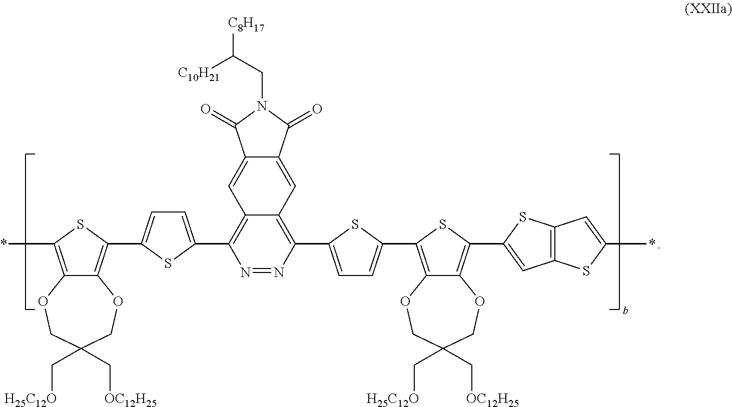

The compound may have the following formula (XXIII):

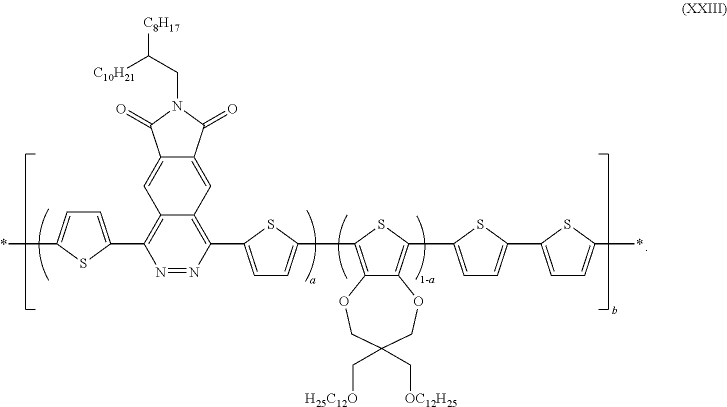

The compound may have the following formula (XXIIIa):

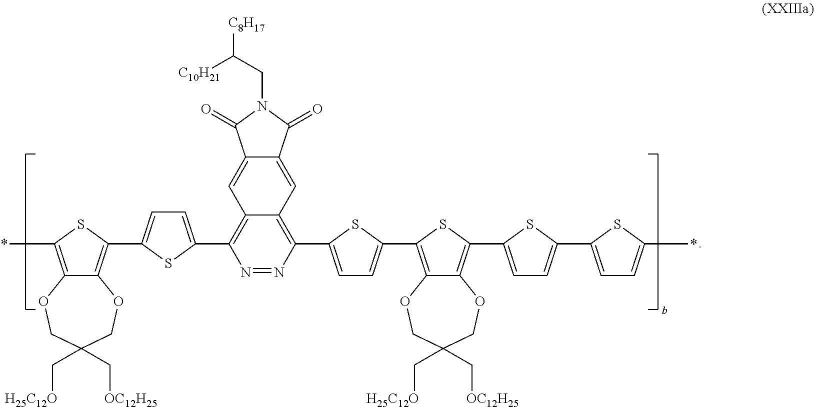

The compound may have the following Formula (XXIVa) or (XXIVb):

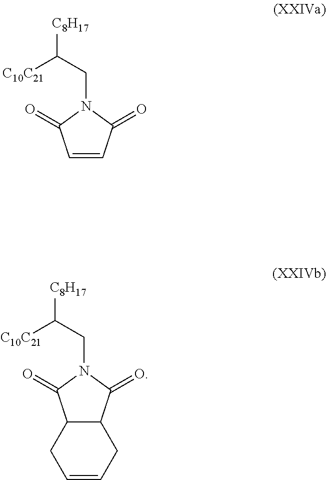

A method for synthesizing the compound of Formula (VI) as defined above may comprise the steps of providing a hydrazine and contacting the hydrazine with a functionalized cyclic group, wherein the cyclic group of the functionalized cyclic group is selected from the group comprising of optionally substituted furan, optionally substituted thiophene, optionally substituted selenophene, optionally substituted tellurophene, optionally substituted pyrrole, optionally substituted phenylene, optionally substituted aza-phenylene, optionally substituted arylene diimide, optionally substituted fluorene, optionally substituted cabazole, optionally substituted dibenzodilole, optionally substituted benzooxodiazole, optionally substituted benzotriazole, optionally substituted benzothidiazole and any combination thereof.

The functionalized cyclic group may be functionalized with at least one nitrile (CN) group.

The functionalized cyclic group may be 2-cyanothiophene.

A method for synthesizing the compound of Formula (I) as defined above may comprise the steps of:
providing the compound of Formula (IV) as defined above; and

contacting the compound of Formula (IV) as defined above with the compound (VIIa) or (VIIb) as defined above under reaction conditions to form the compound of Formula (III) as defined above. The method for synthesizing the compound of Formula (I) as defined above may comprise an inverse-electron demanding Diels-Alder reaction. An inverse-electron demanding Diels-Alder reaction is an organic chemical reaction in which two new chemical bonds and a six-membered ring are formed. It is related to the Diels-Alder reaction, whereby an organic chemical reaction between a conjugated diene and a substituted alkene, commonly termed the dienophile, forms a substituted cyclohexene system. However, unlike the Diels-Alder reaction, the inverse electron demand Diels-Alder reaction is a cycloaddition between an electron-rich dienophile and an electon-poor diene. During an inverse-electron demand Diels-Alder reaction, three pi-bonds are broken and two sigma bonds and one new pi-bond are formed. In the present disclosure, the electronic nature of the reaction partners for an inverse Diels-Alder reaction is inversed again. The tetrazine, as the diene being used herein, may have electron-donating substituents, such as thiophenes, and is therefore considered electron-rich. The dienophile may be an intrinsically electron-poor moiety, such as an imide. This may result in synthetic challenges, which are solved by an inventive choice of solvents and reaction conditions.

The compounds of the formula (XXIVa) and (XXIVb) may both comprise electron withdrawing imides. The compounds of formula (XXIVa) and (XXIVb) may be reacted independently with the compound of Formula (I) to form the compound of Formula (III).

The compound of Formula (VIIa) or (VIIb) may be contacted with the compound of Formula (VI) to form P² in the compound of Formula (I). When the compound of Formula (VIIa) is contacted with the compound of Formula (VI), P² of the compound of Formula (I) may have the Formula (IIIa). When the compound of Formula (VIIb) is contacted with the compound of Formula (VI), P² of the compound of Formula (I) may have the Formula (IIIb).

The method as defined above may be performed under reaction conditions. Reaction conditions may refer to the solvent, reaction temperature and duration of reaction. The choice of solvent, temperature and duration for the reaction for forming the compound of Formula (I) may be very important.

The solvent may be selected from the group consisting of chloroform, dichloromethane, tetrachloromethane, tetrahydrofuran, dioxane, diphenyl ether, toluene, chlorobenzene, dichlorobenzene, xylene and any mixture thereof. The solvent may not contain any reactivity towards the reaction mixture.

The reaction temperature may be in the range of about 40° C. to about 200° C., about 40 ° C. to about 60° C., about 40° C. to about 80° C., about 40° C. to about 100° C., about 40° C. to about 120° C., about 40° C. to about 140° C., about 40° C. to about 160° C., about 40° C. to about 180° C., about 60° C. to about 80° C., about 60° C. to about 100° C., about 60° C. to about 120° C., about 60° C. to about 140° C., about 60° C. to about 160° C., about 60° C. to about 180° C., about 60° C. to about 200° C., about 80° C. to about 100° C., about 80° C. to about 120° C., about 80° C. to about 140° C., about 80° C. to about 160° C., about 80° C. to about 180° C., about 80° C. to about 200° C., about 100° C. to about 120° C., about 100° C. to about 140° C., about 100° C. to about 160° C., about 100° C. to about 180° C., about 100° C. to about 200° C., about 120° C. to about 140° C., about 120° C. to about 160° C., about 120° C. to about 180° C., about 120° C. to about 200° C., about 140° C. to about 160° C., about 140° C. to about 180° C., about 140° C. to about 200° C., about 160° C. to about 180° C., about 160° C. to about 200° C. or about 180° C. to about 200° C. The boiling point of the solvent may be sufficiently high to allow the reaction to proceed.

The duration of reaction may be in the range of about 4 hours to about 24 hours, about 4 hours to about 6 hours, about 4 hours to about 8 hours, about 4 hours to about 10 hours, about 4 hours to about 12 hours, about 4 hours to about 18 hours, about 4 hours to about 24 hours, about 6 hours to about 8hours, about 6 hours to about 10 hours, about 6 hours to about 12 hours, about 6 hours to about 18hours, about 6 hours to about 24 hours, about 8 hours to about 10 hours, about 8 hours to about 12hours, about 8 hours to about 18 hours, about 8 hours to about 24 hours, about 10 hours to about 12hours, about 10 hours to about 18 hours, about 10 hours to about 24 hours, about 12 hours to about 18hours, about 12 hours to about 24 hours or about 18 hours to about 24 hours.

The reaction may proceed efficiently when diphenyl ether is used as the solvent and the reaction temperature is about 160° C. and the reaction duration is about 12 hours. If a solvent with a lower boiling point is used, for example, such that the reaction temperature cannot reach a sufficiently high temperature, the reaction may not proceed efficiently or at all even after 12 hours.

The method may further comprise the step of reacting the compound of Formula (III) as defined above with N-bromosuccinimide. The method may further comprise the step of reacting the compound of Formula (I) as defined above with N-bromosuccinimide and a peroxide, followed by a base when P² of Formula (I) has the Formula (IIIc). When the P² of the compound of Formula (I) has the Formula (IIIc), and this is reacted with N-bromosuccinimide and a peroxide, followed by a base, the resulting compound is the compound of Formula (I) where P² has the Formula (IIId). The method may further comprise the step of reacting the compound of Formula (XVa) as defined above with N-bromosuccinimide and a peroxide, followed by a base, resulting in the formation of the compound of Formula (XVb).

The step of reacting the compound of Formula (I) as defined above with N-bromosuccinimide may be performed in a solvent selected from the group consisting of chloroform, trichloromethane, benzene and any mixture thereof.

The peroxide may be hydrogen peroxide or benzoyl peroxide. The peroxide may be benzoyl peroxide. The base may be triethylamine.

A method for synthesizing the compound of Formula (IV) as defined above may comprise the steps of:
providing the compound of Formula (I) as defined above; and
contacting the compound of Formula (I) as defined above with a compound having the following Formula (XVIIa), (XVIIb) or (XVIIc):

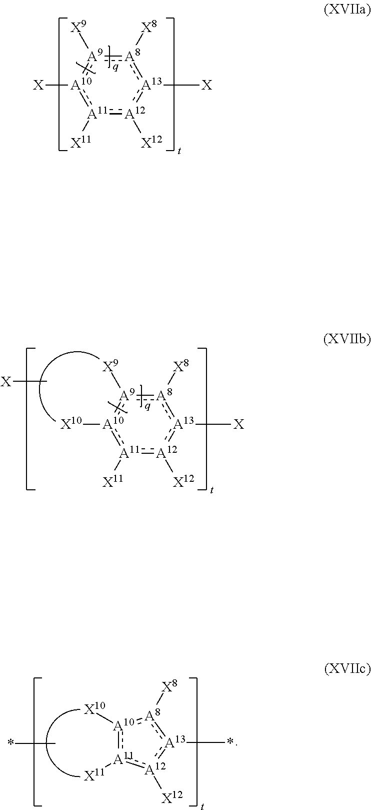

and a cross-coupling reagent comprising a cyclic group; and an element selected from the group consisting of zinc, tin and boron; wherein the cyclic group may be selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl; in the presence of a metal-containing catalyst.

A method for synthesizing the compound of Formula (IV) as defined above, comprising the steps of:
1) providing the compound of Formula (I) as defined above; and
2) contacting the compound of Formula (I) as defined above with a compound having the following Formula (XXVI) in the presence of a metal-containing catalyst to form a monomer precursor:

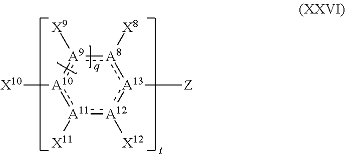

where Z may be a functional group containing zinc, tin or boron. Z may be Sn(CH₃)₃ or Sn(C₄H₉)₃;
3) reacting the monomer precursor of step (2) with N-bromosuccinimide to form a reactive monomer; and
4) reacting the reactive monomer of step (2) with a cross-coupling reagent comprising a cyclic group; and an element selected from the group consisting of zinc, tin and boron; wherein the cyclic group is selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl; in the presence of a metal-containing catalyst.

When q is 0, A⁸ may be S; X⁸ may be absent; A¹⁰, A¹¹, A¹² and A¹³ may be C, Z may be Sn(CH₃)₃ X¹⁰ may be H and X¹¹ and X¹² may be taken together to form an optionally substituted 7-membered heterocycloalkyl. The optionally substituted 7-membered heterocycloalkyl may be substituted with two alkoxy groups. The alkoxy groups may be OC₁₂H₂₅.

The compound of Formula (XXVI) may have the following formula (XXVIa):

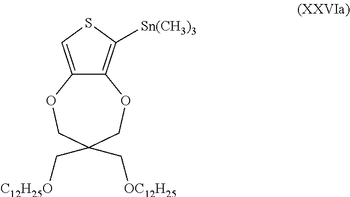

The metal in the metal-containing catalyst may be selected from the group consisting of palladium, nickel and copper. The metal-containing catalyst may be Pd(PPh₃)₄.

The cross-coupling reagent may comprise tin. The cross-coupling reagent may comprise boron.

The cross-coupling reagent comprising tin may have the following formula (XXVII):

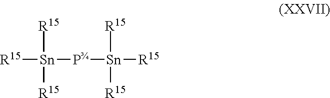

wherein R¹⁵ may be an optionally substituted alkyl.

The cross-coupling reagent comprising boron may have the following formula (XXVIIIa);

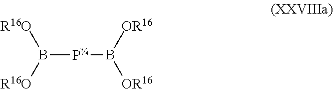

wherein R¹⁶ may be H or optionally substituted alkyl.

The cross-coupling reagent comprising boron may have the following formula (XXVIb) or (XXVIc):

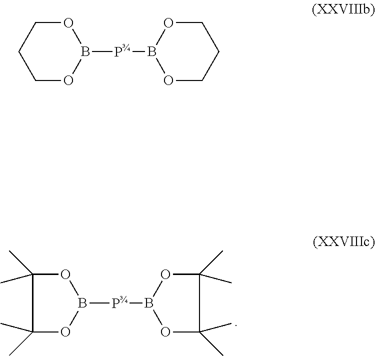

P^{3/4} may refer to P³ or P⁴.

The cross-coupling reagent may have the following Formula (XXIXa), (XXIXb) or (XXIXc):

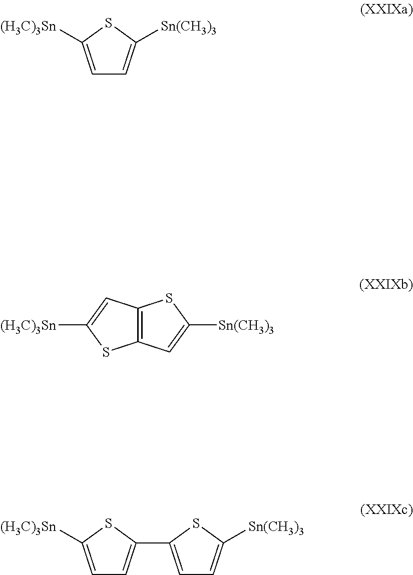

The cross-coupling reagent may have the following Formula (XXX):

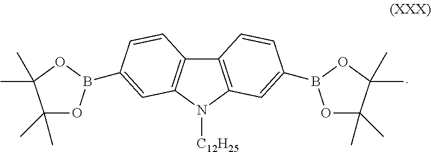

Use of the compound as defined above may be in the manufacture of semiconductor devices. A semiconductor device may comprise the compound of Formula (IV) as defined above.

### EXAMPLES

Non-limiting examples of the disclosure and a comparative example will be further described in greater detail by reference to specific Examples, which should not be construed as in any way limiting the scope of the invention.

The synthetic steps are disclosed in a general manner in the following synthetic methods. Variations of these general methods in order to produce the polymers according to the present disclosure are within the ability of the person skilled in the art. Where representative procedures are given, these can be adjusted by the person skilled in the art to other derivatives, if needed.

### EXAMPLE 1

### Synthesized Monomers

Pyridazines

Pyridazines synthesized having the following structure are shown in Table 1:

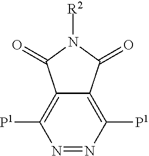

TABLE 1
Pyradazine monomers

| Monomer name | P¹ | R² |
|---|---|---|
| Monomer 1 | 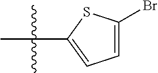 [SMILES: *c1ccc(Br)s1] | hexyl |
| Monomer 2 | 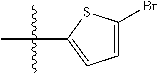 [SMILES: *c1ccc(Br)s1] | dodecyl |
| Monomer 3 | 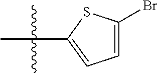 [SMILES: *c1ccc(Br)s1] | 2-octyldodecyl |
| Monomer 4 | 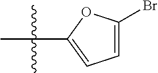 [SMILES: *c1ccc(Br)o1] | hexyl |
| Monomer 5 | 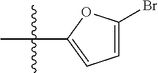 [SMILES: *c1ccc(Br)o1] | dodecyl |
| Monomer 6 | 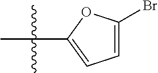 [SMILES: *c1ccc(Br)o1] | 2-octyldodecyl |
| Monomer 7 | 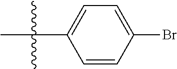 [SMILES: *c1ccc(Br)cc1] | hexyl |
| Monomer 8 | 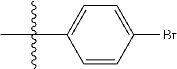 [SMILES: *c1ccc(Br)cc1] | dodecyl |
| Monomer 9 | 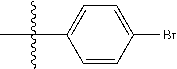 [SMILES: *c1ccc(Br)cc1] | 2-octyldodecyl |

Phthalazines

Phthalazines synthesized having the following structure are shown in Table 2:

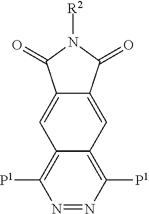

TABLE 2
Phthalazine monomers

| Monomer name | P¹ | R² |
|---|---|---|
| Monomer 10 | 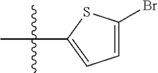 [SMILES: *c1ccc(Br)s1] | butyl |
| Monomer 11 | 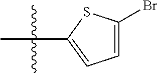 [SMILES: *c1ccc(Br)s1] | 2-octyldodecyl |
| Monomer 12 | 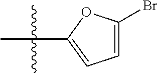 [SMILES: *c1ccc(Br)o1] | butyl |
| Monomer 13 | 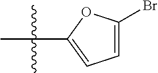 [SMILES: *c1ccc(Br)o1] | 2-octyldodecyl |

### EXAMPLE 2

Some polymers synthesized from selected monomers in Tables 1 and 2 are shown in Table 3.

TABLE 3
Synthesized Polymers

| | |
|---|---|
| Polymer 1 | 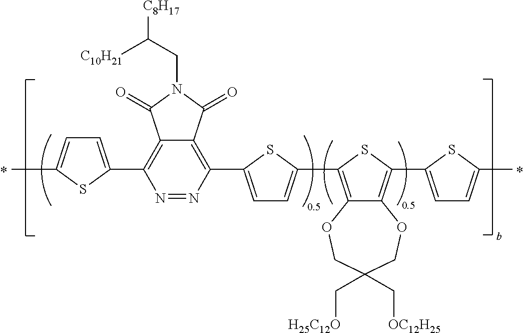 |
| Polymer 2 | 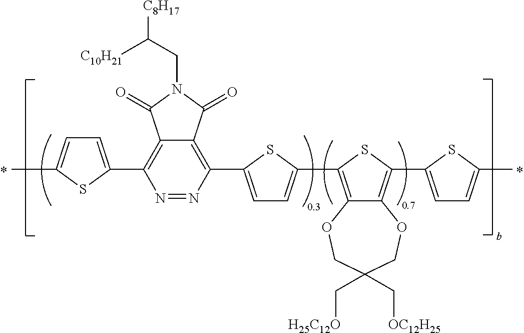 |
| Polymer 3 | 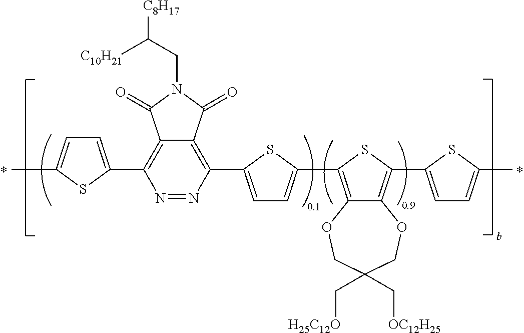 |
| Polymer 4 | 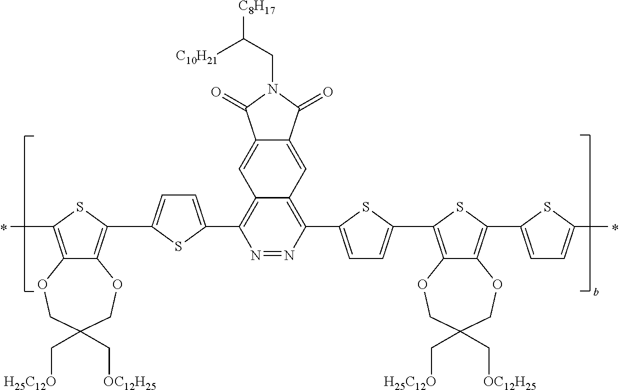 |
| Polymer 5 | 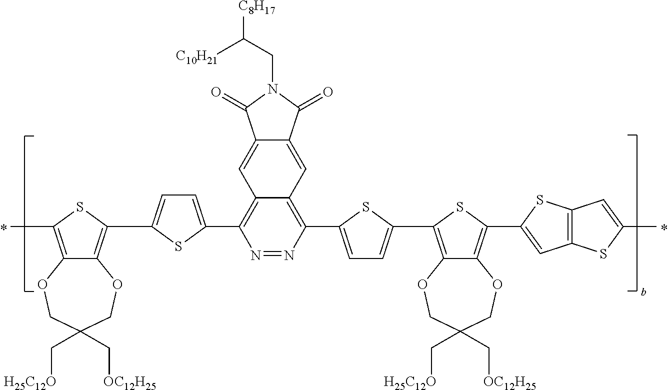 |
| Polymer 6 | 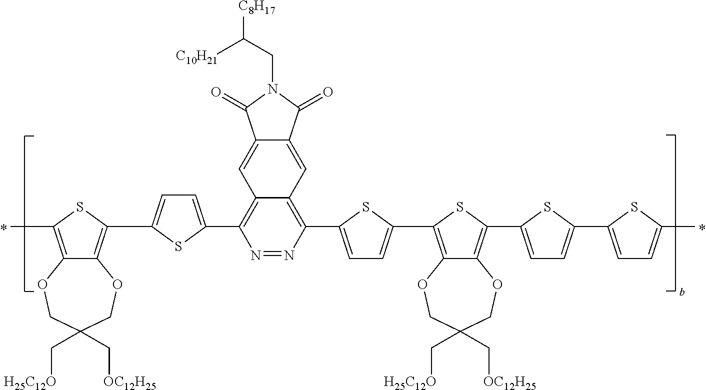 |
| Polymer 7 | 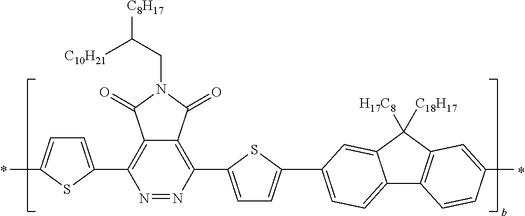 |
| Polymer 8 | 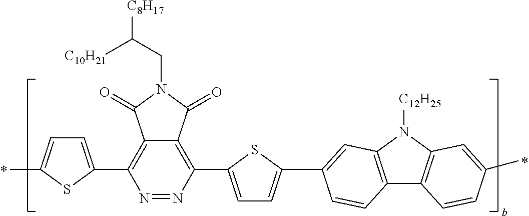 |
| Polymer 9 | 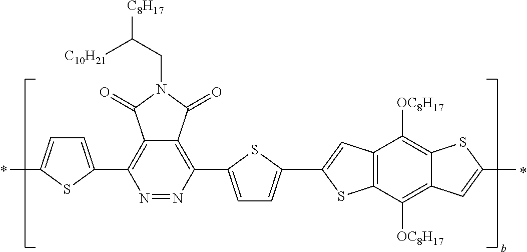 |
| Polymer 10 | 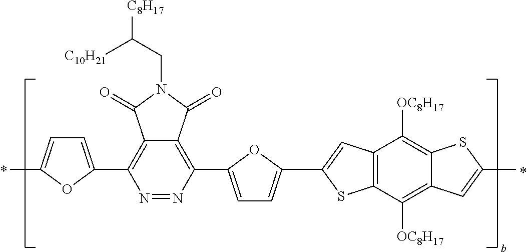 |
| Polymer 11 | 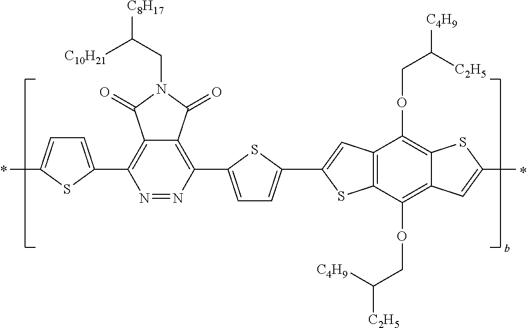 |
| Polymer 12 | 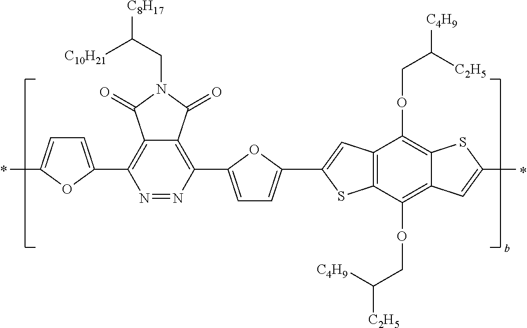 |

For all of polymers 1 to 12, b is an integer from 1 to 50.

### EXAMPLE 3

### Synthesis of the Tetrazine

The synthesis of a tetrazine precursor 1 is illustrated below:

3,6-Di(thiophen-2-yl)-1,2,4,5-tetrazine:

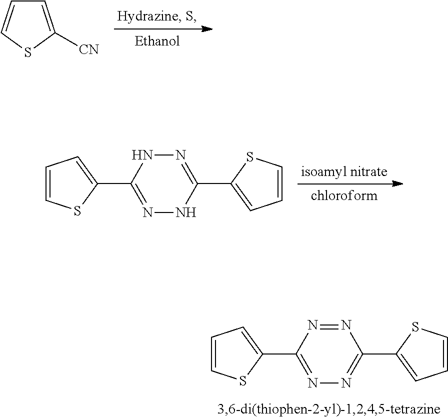 [SMILES: N#Cc1cccs1 ; c1csc(C2=NNC(c3cccs3)=NN2)c1 ; c1csc(-c2nnc(-c3cccs3)nn2)c1]

To a dried round bottom flask (1 L) was added 2-cyanothiophene (50 grams, 0.4587 mol), hydrazine (30% concentration, 50 mL), sulphur powder (1.47 grams, 0.04587 mol) and ethanol (200 mL). The mixture was stirred at 70° C. for 3 hours before cooling down to room temperature. Brown crystals were formed at the bottom of the solution. The crystals were collected by suction filtration and washed with cold ethanol (100 mL). After complete drying, the collected crystal was loaded into a dry round bottom flask (1 L). Isoamyl nitrite (107 grams, 0.9174 mol) and chloroform (200 mL) were added into the round bottom flask. The mixture was stirred at room temperature for 6 hours. The solvent was removed by rotary evaporation and the remaining solid was recrystallized in toluene to afford the target compound as a pure red crystal. Yield=47 grams, 84%.

### EXAMPLE 4

### Synthesis of Polymer 1, Polymer 2 and Polymer 3

General Synthesis of the Diels-Alder Product

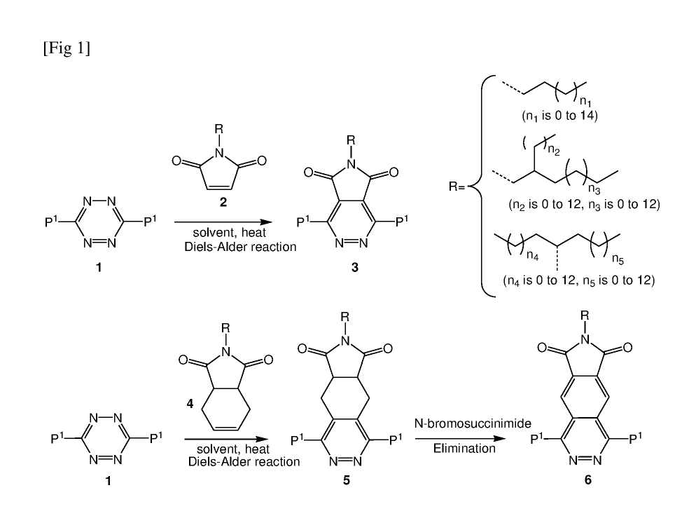

The general synthetic scheme for the monomer synthesis is provided in FIG. 1. A tetrazine (compound (I) in the detailed description, compound 1 in FIG. 1) was subjected to a Diels-Alder reaction with a dienophile. The dienophile may be a representative of the structure Vila, as shown in compound 2 to yield compound 3. The dienophile may be a representative of the structure VIIb, as shown in compound 4, to yield, compound 5. Compound 5 may undergo a further elimination step, effected by treatment with N-bromo succinimide and dibenzoyl peroxide. A basic work-up then affords compound 6. Compounds 3 and 6 may be further manipulated or used directly as monomers for the polymer synthesis.

Diels-Alder Reaction—General Procedure for the Synthesis of the Compounds 3 and 5

To a dry round bottom flask was added the functionalized tetrazine compound (10 mmol), the dienophile moiety (10 mmol) and appropriate solvent. The solvent may be CHCl₃, DCM, THF, dioxane, toluene, chlorobenzene, dichlorobenzene, xylene, etc. The mixture was heated to reflux overnight. After it was cooled down, the mixture was poured into cold methanol and the precipitate was collected via suction filtration. It was then thoroughly washed with cold methanol and used directly for the next step without further purification.

Elimination Step—General Procedure for the Synthesis of the Compound 6

To a dry round bottom flask was added the substrate (8 mmol), NBS (16 mmol) and appropriate solvent (chloroform, tetrachloromethane, benzene etc.), benzoyl peroxide (1 mmol). The mixture was heated to reflux overnight. After cooling down to room temperature, the mixture was poured into methanol. The precipitate was collected and used directly for the next step without further purification to yield the brominated product.

To a dry round bottom flask was added the brominated product (4 mmol), THF (10 ml) and triethylamine (10 ml). The mixture was stirred at room temperature overnight. The organic solvent was then removed and the target molecule was obtained by silica gel column chromatography.

The Stille Cross-Coupling Reaction for Polymer 1, Polymer 2 and Polymer 3

1,4-bis(5-bromothiophen-2-yl)-6-(2-octyldodecyl)-5H-pyrrolo[3,4-d]pyridazine-5,7(6H)-dione (24 mg, 0.032 mmol), 6,8-dibromo-3,3-bis((dodecyloxy)methyl)-3,4-dihydro-2H-thieno[3,4-b][1,4]dioxepine (200 mg, 0.281 mmol), and 2,5-bis(trimethylstannyI)thiophene(128 mg, 0.312 mmol) were dissolved in 12 ml of toluene, The mixture was stirred and purged with Argon for about 15 min, and then Pd(PPh₃)₄ (18.1 mg, 0.016 mmol) was added. The flask was purged with Argon for another 15 min before the mixture was heated to 110° C. for 48 hrs. Excess solvent was removed under reduced pressure and the polymer was precipitated with methanol. The suspension was filtered to give the crude product, which was then purified by Soxhlet extraction with acetone and chloroform. The chloroform fraction was evaporated to dryness to afford a purple-red solid.

The following stoichiometric ratio relative to 2,5-bis(trimethylstannyI)thiophene was used in the polymers:

TABLE 4
Stoichiometric ratios of starting material in Polymers 1, 2 and 3

| | 1,4-bis(5-bromothiophen-2- | 6,8-dibromo-3,3- |
|---|---|---|
| | yl)-6-(2-octyldodecyl)-5H- | bis((dodecyloxy)methyl)-3,4- |
| | pyrrolo[3,4-d]pyridazine- | dihydro-2H-thieno[3,4- |
| | 5,7(6H)-dione | b][1,4]dioxepine |
| Polymer 1 | 0.5 | 0.5 |
| Polymer 2 | 0.7 | 0.3 |
| Polymer 3 | 0.9 | 0.1 |

### EXAMPLE 5

### Characterization of Polymer 1, Polymer 2 and Polymer 3

¹H NMR Characterization

Polymer 1. Yield 68%. ¹H NMR (main signals) (CDCl₃): δ 7.16-7.07 (br, s), 4.22 (br, s), 3.63-3.43 (br, m), 1.25 (br, s), 0.87 (br, s).

Polymer 2. Yield 70%. ¹H NMR (main signals) (CDCl₃); δ 7.06-7.00 (br, s), 4.16 (br, s), 3.62-3.42 (br, m), 1.25 (br, s), 0.88 (br, s).

Polymer 3. Yield 50%. ¹H NMR (main signals) (CDCl₃): δ 7.17-7.07 (br, s), 4.22 (br, s), 3.62-3.45 (br, m), 1.25 (br, s), 0.88 (br, s).

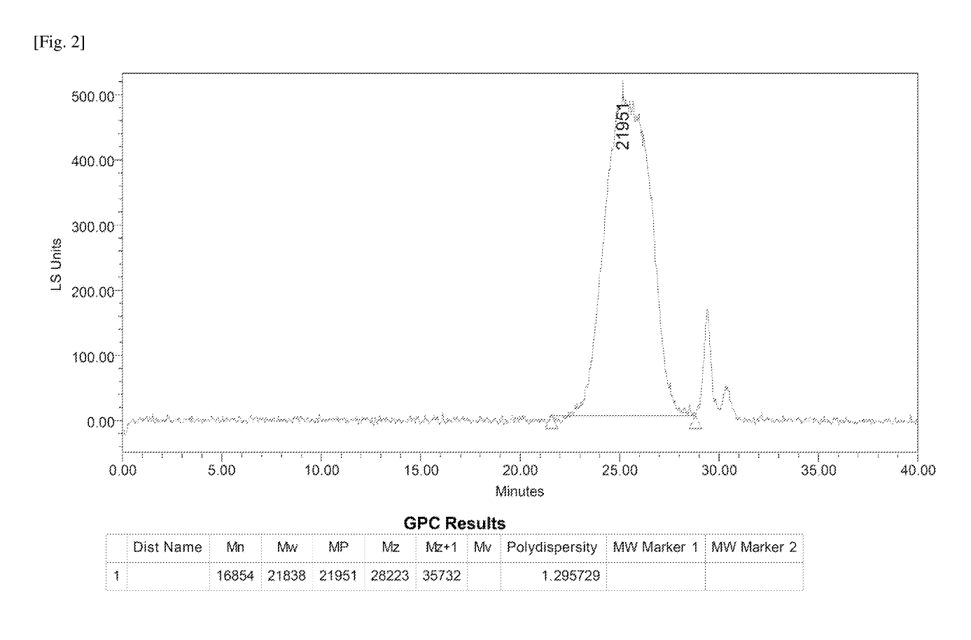

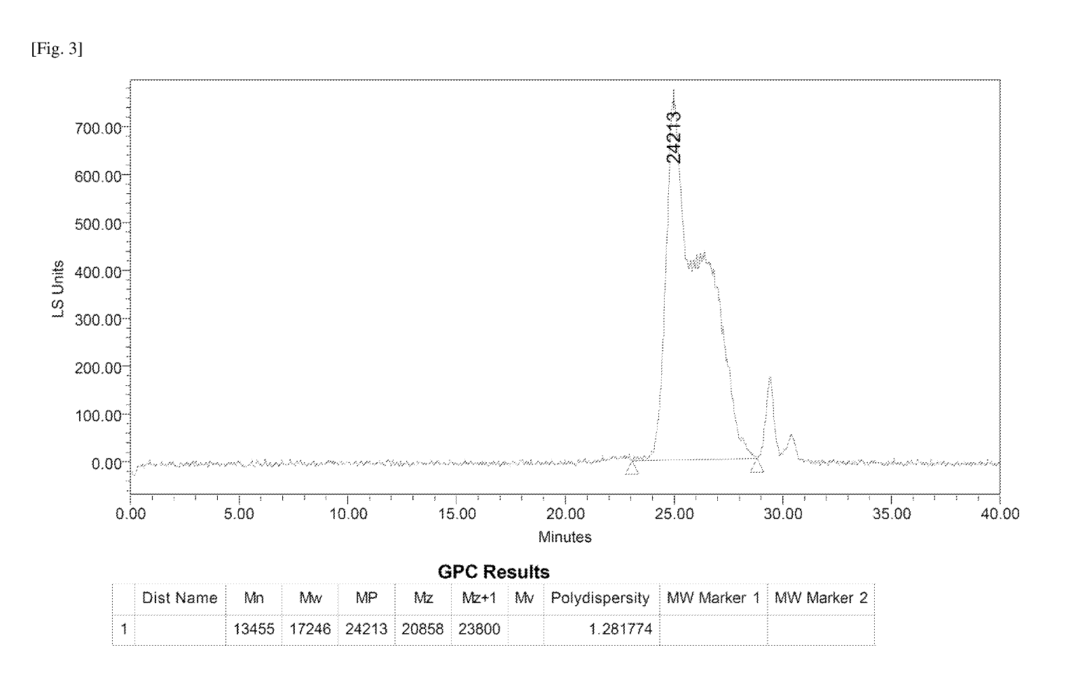

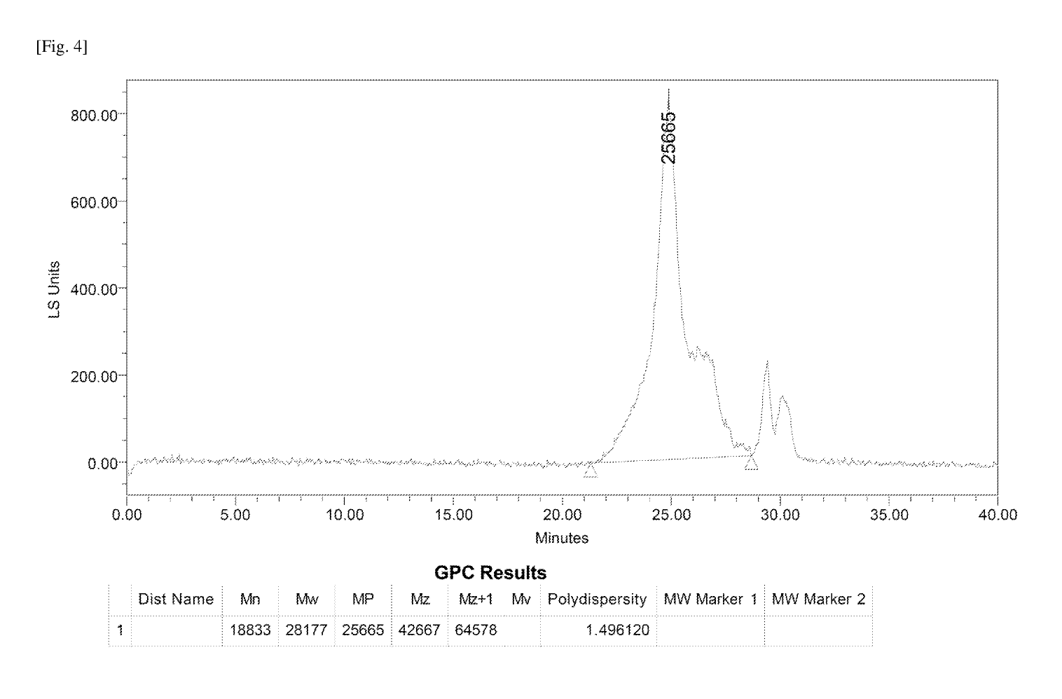

The GPC curves of these three polymers are shown in FIGS. 2, 3 and 4. Gel permeation chromatography technique is commonly used to determine the molecular weight of the conjugated polymers. By reading the retention time of the polymer peak, the molecular weight can be determined according to the calibration data of some standard polymers.

UV-Vis Absorbance

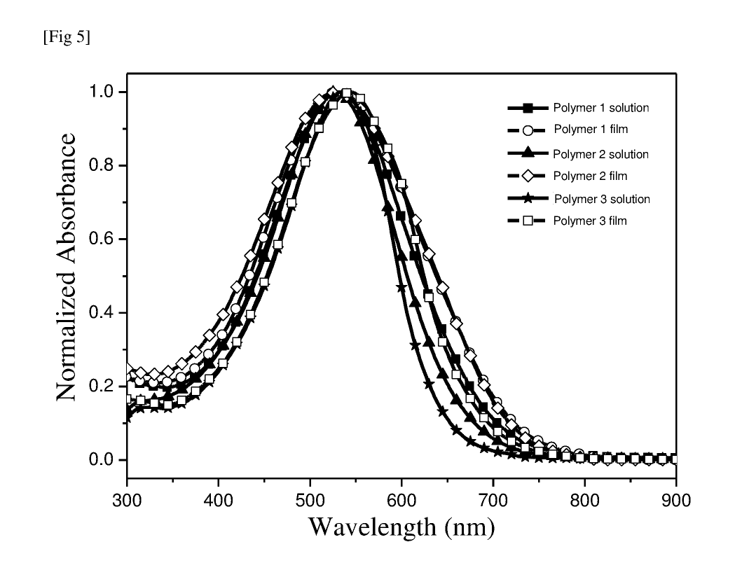

The UV absorptions of solutions of the polymer 1, polymer 2 and polymer 3 in chlorobenzene in the wavelength range of 300 to 900 nm were recorded and are shown in FIG. 5.

### EXAMPLE 6

### Manufacture of Devices Using Polymer 1, Polymer 2 and Polymer 3

Indium tin oxide (ITO)/glass (15Ω/sq, 35×30×1.1mm) were used as substrates. The substrates were cleaned by successive sonication in acetone, isopropyl alcohol and DI water for 15 minutes each. They were then blown dry using nitrogen gas. Polymer solutions of polymers 1-3 were prepared at a concentration of 10 mg/ml using a mixed solvent system chloroform and chlorobenzene in a 6:4 volume ratio. The solutions were filtered through a 0.45 μm PVDF syringe filter prior to use. 80 μL of the polymer solution was pipetted onto the ITO/glass substrates and a polymer film was formed from spin-coating at 500 rpm for 60 seconds. The excessive polymer edges were removed by swabbing with chloroform using a cotton bud to obtain an active area of 2×2 cm². On a second piece of cleaned ITO/glass substrate, an area of 2×2 cm² was blocked out using parafilm. The total thickness of the parafilm spacer was kept constant at 0.01″. 250 μL of the gel electrolyte (0.512 g of lithium perchlorate and 2.8 g of poly(methyl methacrylate) (MW=120,000 g/mol) in 6.65 ml of propylene carbonate and 28 ml of dry acetonitrile) was pipetted within the 2×2 cm² area and left to dry for 5 minutes.

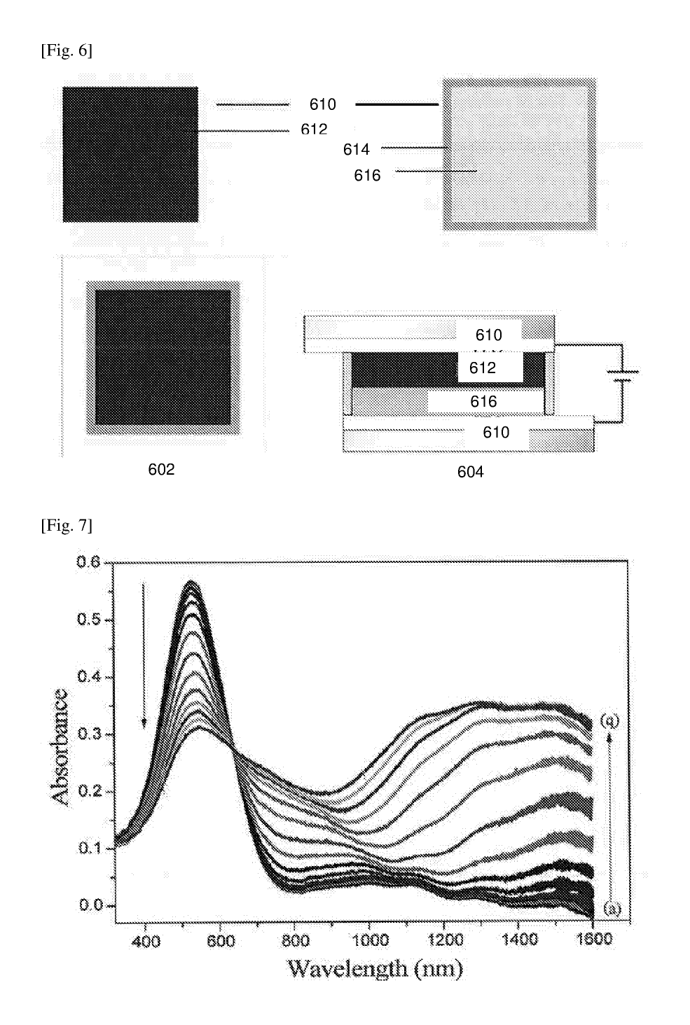

The device was fabricated by assembling the two ITO/glass substrates together with the polymer film and gel electrolyte in contact. The device was secured using parafilm. A schematic depiction of the device thus produced is shown in FIG. 6. It shows the different layers of ITO/glass substrate with gel electrolyte and the polymer, as a top view (602) as well as a cross section (604). The ITO/glass substrate comprises the ITO/glass (610), polymer (612), parafilm (614) and gel electrolyte (616).

### EXAMPLE 7

### Characterization of Devices Manufactured Using Polymer 1, Polymer 2 and Polymer 3

UV-Vis Absorbance

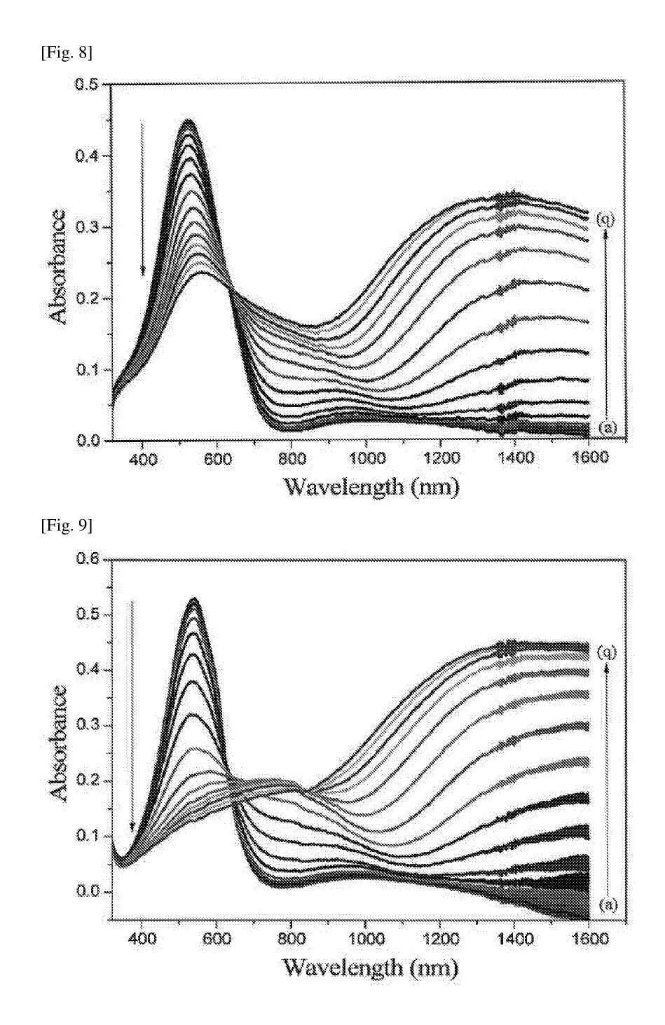

The UV-vis-NIR absorption spectra of the polymer devices at various applied potentials (V) for the polymer 1, polymer 2 and polymer 3 are recorded and shown in FIG. 7, FIG. 8 and FIG. 9. They demonstrate the dependence of the respective electronic transition states to an applied potential. When different voltage is applied on the polymer thin film, the film absorption the light differently and therefore there will be a color change on the color window of the electrochromic device.

Square-wave Potential Step Absorbance

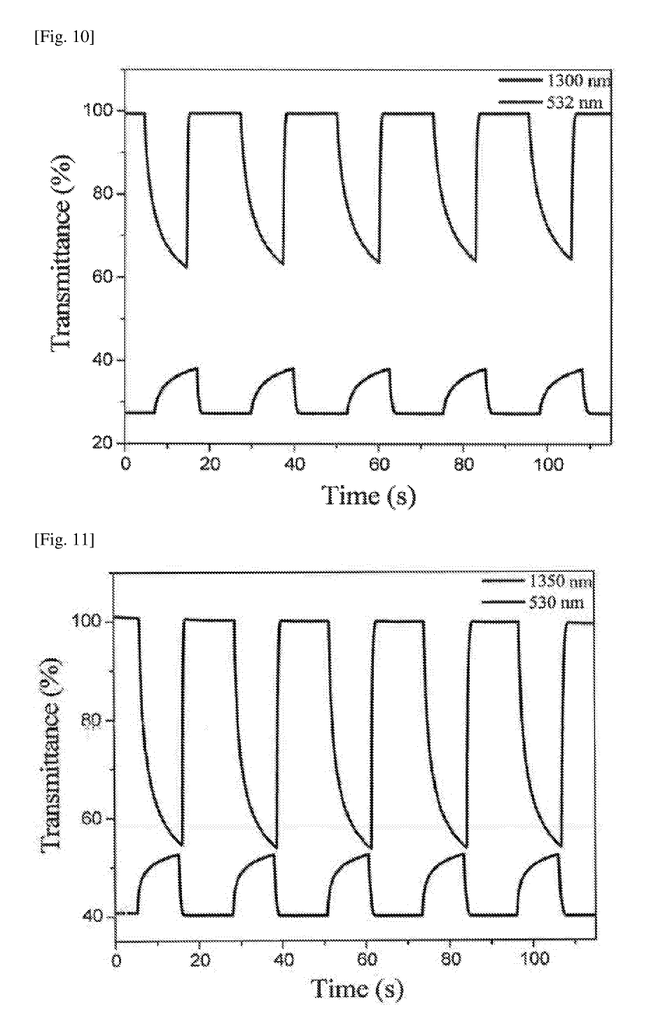

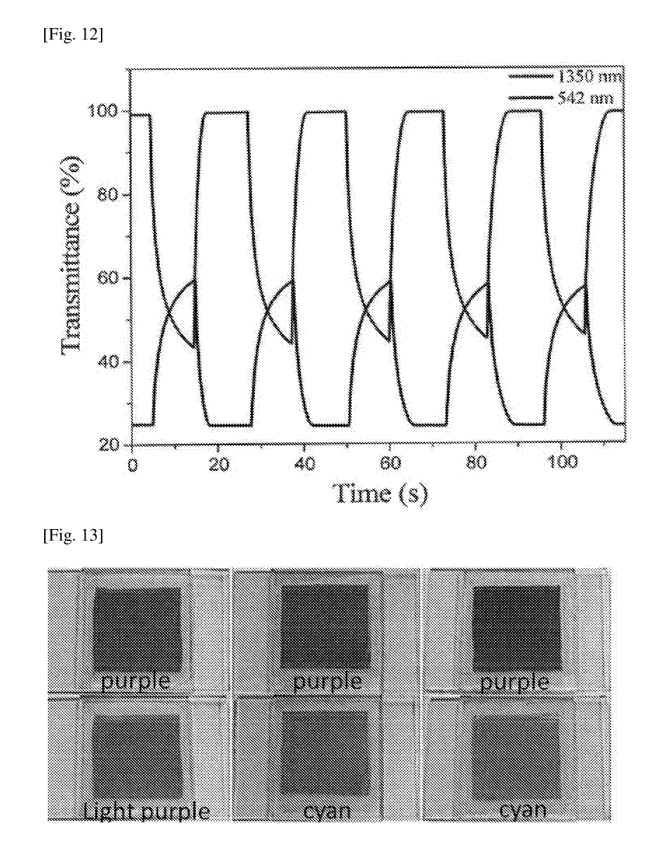

The square-wave potential step absorptiometry of the polymer devices between 1.5 V and −1.5 V with a switch time of 10 seconds were recorded for the polymer 1, polymer 2 and polymer 3 and shown in FIG. 10, FIG. 11 and FIG. 12. They demonstrate dependence of the respective electronic transition states to an applied potential and reversibility of the process. Switching time, bleaching time and the stability of the electrochromic device can be extracted from these data.

Optical and Electrochemical Properties

FIG. 13 shows photos of the polymer devices of the polymer 1, polymer 2 and polymer 3 in neutral state and under 2.0 V.

The optical and electrochemical properties of the polymers are shown in Table 5:

TABLE 5
Optical and Electrochemical Properties of Polymers

| | λmax, | λonset, | λmax, | λonset, | Optical | | |
|---|---|---|---|---|---|---|---|
| | solution | solution | film | film | band gap | HOMO | LUMO |
| | (nm) | (nm) | (nm) | (nm) | (eV) | (eV) | (eV) |
| Polymer 1 | 532 | 713 | 530 | 732 | 1.74 | −5.00 | −3.26 |
| Polymer 2 | 528 | 690 | 530 | 724 | 1.80 | −4.95 | −3.15 |
| Polymer 3 | 533 | 660 | 544 | 699 | 1.88 | −4.92 | −3.04 |

The performance of the electrochromic devices in visible light is shown in Table 6.

TABLE 6
Performance of Electrochromic Devices in Visible light

| | | | | Coloration |
|---|---|---|---|---|
| | Contrast | Bleaching | Coloration | Efficiency |
| | (%) | Time (s) | Time (s) | (cm²/C.) |
| Polymer 1 | 10.9 ± 0.1 | 8.30 ± 0.16 | 0.92 ± 0.07 | 331 ± 4 |
| Polymer 2 | 12.4 ± 0.2 | 7.47 ± 0.39 | 0.87 ± 0.06 | 282 ± 15 |
| Polymer 3 | 34.1 ± 0.51 | 7.62 ± 0.26 | 2.38 ± 0.24 | 436 ± 12 |

*Average values and standard deviations are reported based on 3 trials. Coloration refers to the process in which the percent transmittance changes from a higher value to a lower value. Bleaching refers to the process in which the percent transmittance changes from a lower value to a higher value.

The performance of the electrochromic devices in Near Infra Red (NIR) is shown in Table 7.

TABLE 7
Performance of Electrochromic Devices in Near Infra Red (NIR)

| | | | | Coloration |
|---|---|---|---|---|
| | Contrast | Bleaching | Coloration | Efficiency |
| | (%) | Time (s) | Time (s) | (cm²/C.) |
| Polymer 1 | 36.2 ± 1.2 | 0.66 ± 0.11 | 8.06 ± 0.18 | 454 ± 35 |
| Polymer 2 | 47.2 ± 1.5 | 0.85 ± 0.06 | 7.35 ± 0.14 | 453 ± 5 |
| Polymer 3 | 54.2 ± 1.4 | 2.24 ± 0.24 | 7.12 ± 0.42 | 457 ± 14 |

*Average values and standard deviations are reported based on 3 trials. Coloration refers to the process in which the percent transmittance changes from a higher value to a lower value.

Bleaching refers to the process in which the percent transmittance changes from a lower value to a higher value.

### EXAMPLE 8

### Synthesis of Polymer 4, Polymer 5 and Polymer 6

General Synthetic Scheme

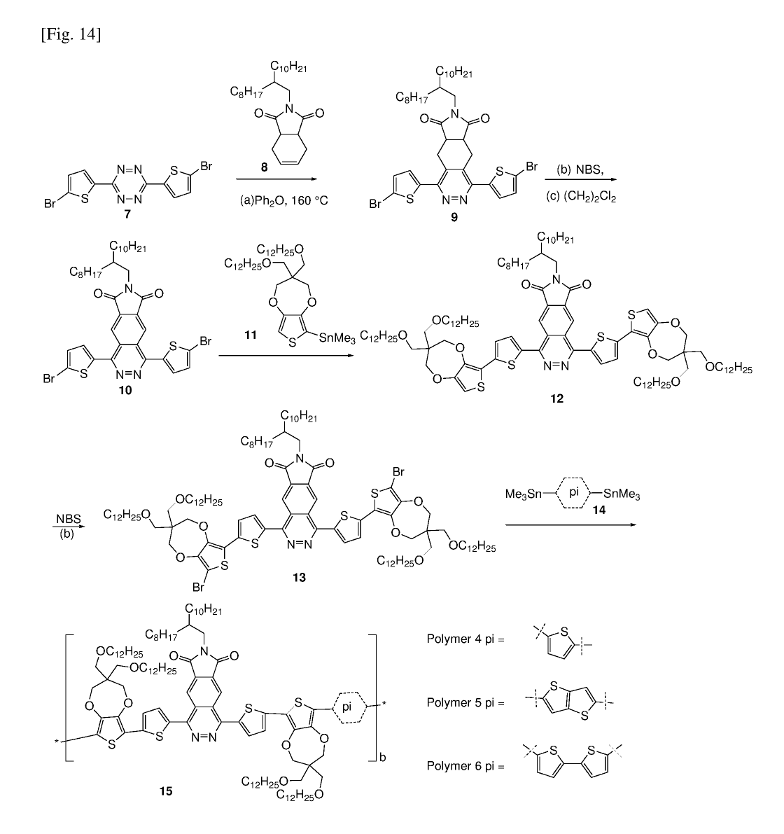

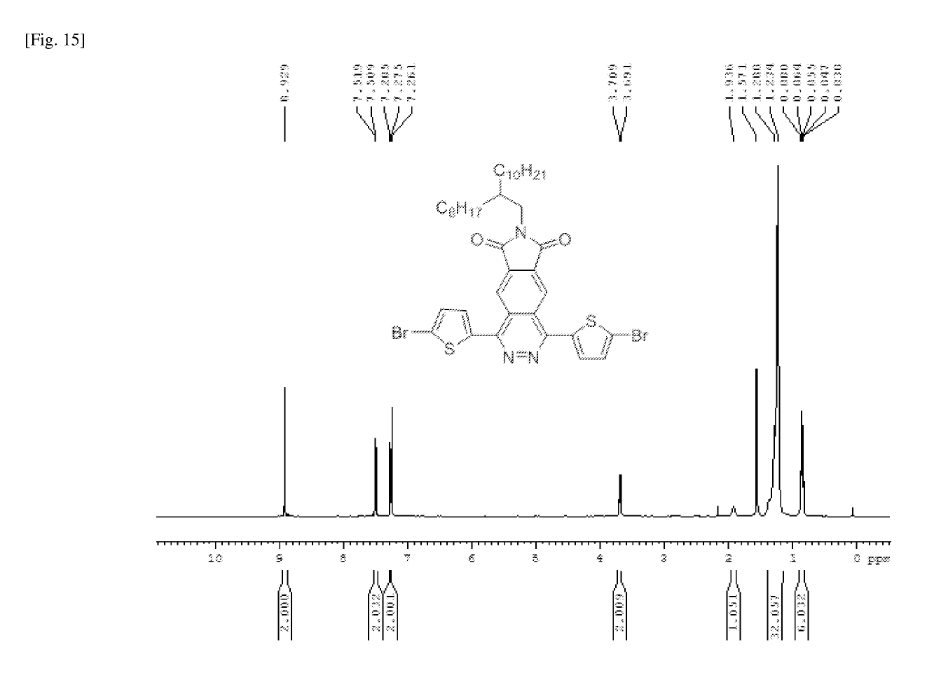

The general synthetic scheme is provided in FIG. 14, the following reactions are undertaken in order to make the polymer 4, polymer 5 and polymer 6.

Diels-Alder Reaction (Synthesis of Compound 9)

1,4-Bis(5-bromothiophen-2-yl)-7-(2-octyldodecyl)-5,5a,8a,9-tetrahydro-6H-pyrrolo[3,4-g]phthalazine-6,8(7H)-dione (9).

To a dry round bottom flask was added 3,6-bis(5-bromothiophen-2-yl)-1,2,4,5-tetrazine (7) (3 g, 7.43 mmol), 2-(2-octyldodecyl)-3a,4,7,7a-tetrahydro-1H-isoindole-1,3(2H)-dione (8) (3.19 g, 7.43 mmol) and diphenyl ether (a) (20 ml). The mixture was purged with argon for 15 min and then heated to 160° C. for 16 h. After cooling to room temperature, the mixture was directly subjected to silica gel column chromatography. Hexane was first used to flush away the diphenyl ether. The target compound was then collected as yellow oil using mixture solvent (Hex:CHCl₃=1:1) then CHCl₃ as eluent (5.10 g, 82%). The product was contaminated by ca. 5% of starting material. The crude product was used for the next step without further purification. HR-APCI-MS: m/z=804.1870, calculated exact mass: 804.1862, error: 0.9 ppm.

Elimination Reaction (Synthesis of Compound 10)

1,4-Bis(5-bromothiophen-2-yl)-7-(2-octyldodecyl)-6H-pyrrolo[3,4-g]phthalazine-6,8(7H)-dione (10).

To a dry round bottom flask was added 1,4-bis(5-bromothiophen-2-yl)-7-(2-octyldodecyl)-5,5a,8a,9-tetrahydro-6H-pyrrolo[3,4-g]phthalazine-6,8(7H)-dione (9) (1 g, 1.25 mmol), NBS (b) (2.2 equiv. 490 mg, 2.75 mmol), benzoyl peroxide (0.1 equiv, 0.125 mmol, 30 mg) and dichloroethane (c) (20 ml). The mixture was purged with argon for 15 min and then heated to 120° C. for 2 days. After cooling to room temperature, the solvent was removed by rotary evaporation. The residue was directly subjected to silica gel column chromatography to afford the target molecule as a yellow powder (720 mg, 72%). ¹H NMR (400 MHz, CDCl₃): δ ppm=8.93 (s, 2H), 7.52-7.51 (d, 2H, J=4 Hz), 7.28-7.27 (d, 2H, J=4 Hz), 3.71-3.69 (d, 2H, J=7.2 Hz), 1.94 (m, 1H), 1.29-1.23 (m, 32H), 0.88-0.84 (m, 6H). ¹³C NMR (100 MHz, CDCl₃): δ ppm=166.84, 152.67, 140.05, 134.21, 131.63, 131.41, 127.56, 122.31, 119.06, 43.60, 37.41, 32.28, 31.89, 30.33, 30.02, 29.69, 26.66, 23.06, 14.50. HR-EI-MS: m/z=801.1470, calculated exact mass: 801.1456, error: 1.81 ppm.

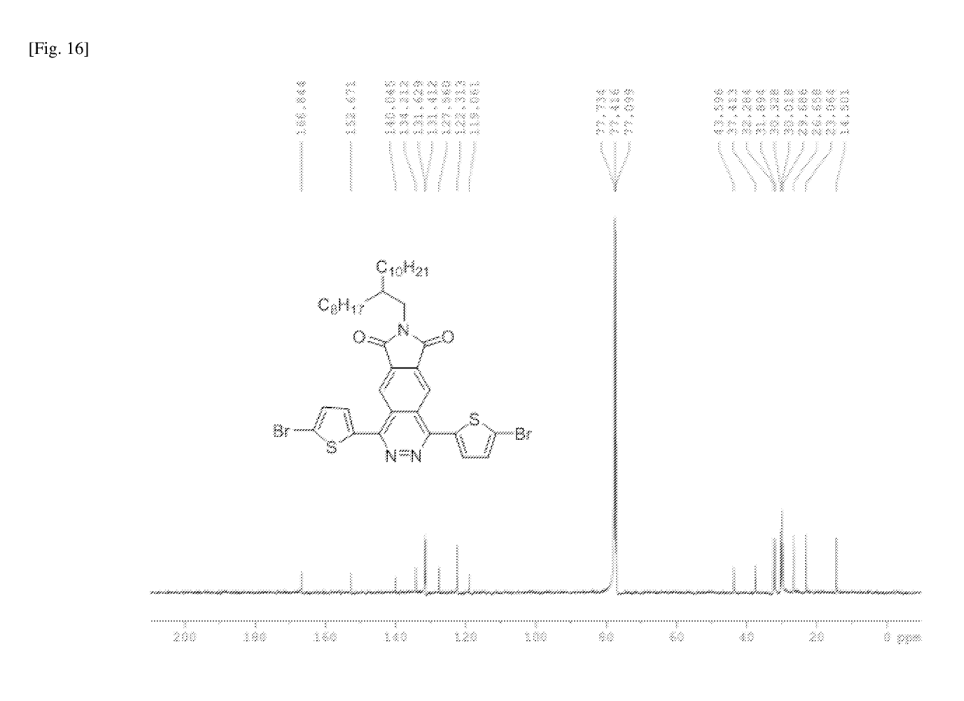

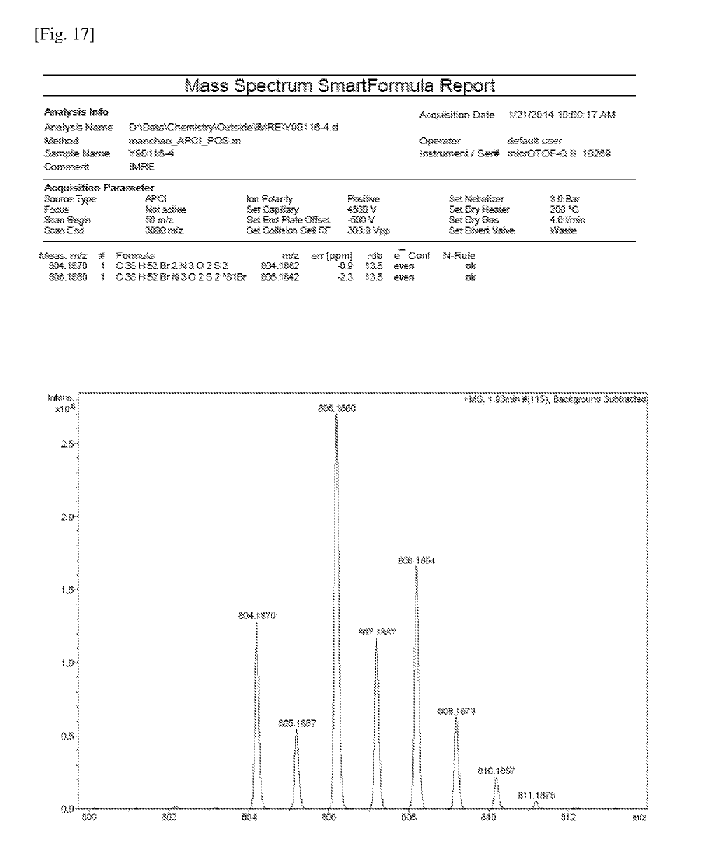

The ¹H NMR, ¹³C NMR and HRMS spectra of this compound (compound 10) are provided in FIG. 15, FIG. 16 and FIG. 17, respectively.

Cross-Coupling Reaction (Synthesis of Compound 12)

1,4-Bis(5-(3,3-bis((dodecyloxy)methy)-3,4-dihydro-2H-theino[3,4-b][1,4]dioxepin-6-yl)thiophene-2-yl)-7-(2-octyldodecyl)-6H-pyrrolo[3,4-g]phthalazine-6,8(7H)-dione (12).

To a dry round bottom flask was added (3,3-bis((dodecyloxy)methyl-3,4-dihydro-2H-thieno[3,4-b][1,4]dioxepin-6-yl)trimethylstannane (11) (2 g, 50% purity), 1,4-bis(5-bromothophen-2-yl)-7-(2-octyldodecyl)-6H-pyrrolo[3,4-g]phthalazine-6,8(7H)-dione (10) (370 mg, 0.47 mmol), Pd(PPh₃)₄ (0.1 equiv, 0.047 mmol, 54 mg) and dry toluene (15 ml). The mixture was degassed by purging with argon and then heated to 110° C. for 16 h. After cooling to room temperature, the solvent was removed by rotary evaporation. The collected residue was dissolved in minimum amount of chloroform and directly subjected to silica gel column chromatography. The target compound was then collected as red waxy solid using a solvent mixture (Hex:CHCl₃=1:1) (510 mg, 62%). The target compound was found to be unstable at ambient condition. It was stored in the fridge after preparation. ¹H NMR (400 MHz, CDCl₃): δ ppm=9.00 (s, 2H), 7.68 (d, 2H, J=3.6 Hz),7.38-7.37 (d, 2H, J=4 Hz), 6.45 (s, 2H), 4.22 (s, 4H), 4.10 (s, 4H), 3.71-3.69 (d, 2H, J=7.2 Hz), 3.55 (m, 8H), 3.44-3.41 (m, 8H), 1.94 (m, 1H), 1.68 (m, 4H), 1.56-1.54 (m, 8H), 1.30-1.24 (m, 100H), 0.88-0.85 (m, 18H). ¹³C NMR (100 MHz, CDCl₃): δ ppm=167.06, 152.76, 150.41, 147.06, 141.15, 136.18, 134.00, 131.31, 127.99, 123.98, 122.81, 116.59, 103.85, 74.41, 72.20, 70.00, 48.25, 43.53, 37.41, 32.33, 31.89, 30.36, 30.08, 29.97, 29.77, 26.58, 23.09, 14.52. HR-APCI-MS: m/z=1744.1423, calculated exact mass: 1744.1411, error: 0.67 ppm.

Bromination Reaction (Synthesis of Compound 13)

1,4-Bis(5-(8-bromo-3,3-bis((dodecyloxy)methyl)-3,4-dihydro-2H-thieno[3,4-b][1,4]dioxepin-6-yl)-7-(2-octyldodecyl)-6H-pyrrolo[3,4-g]-phthalazine-6,8(7H)-dione (13).

To a dry round bottom flask was added 1,4-Bis(5-(3,3-bis((dodecyloxy)methy)-3,4-dihydro-2H-theino[3,4-b][1,4]dioxepin-6-yl)thiophene-2-yl)-7-(2-octyldodecyl)-6H-pyrrolo[3,4-g]phthalazine-6,8(7H)-dione (12) (1 g, 0.573 mmol), NBS (b) (2.2 equiv., 1.26 mmol, 225 mg) and chloroform (10 ml). The mixture was stirred at room temperature overnight. The solvent was removed by rotary evaporation and the residue was directly subjected to silica gel chromatography using hexane: chloroform (1:1) to afford the target product as a red waxy solid (0.92 g, 85%). NMR (400 MHz, CDCl₃): δ ppm=8.98 (s, 2H), 7.66-7.65 (d, J=4 Hz, 2H), 7.30-7.29 (d, J=4 Hz, 2H), 4.22 (s, 4H), 4.17 (s, 4H), 3.71-3.69 (d, J=7.2 Hz, 2H), 3.55 (s, 8H), 3.44-3.41 (t, J=6.4 Hz, 8H), 1.91 (m, 1H), 1.55-1.24 (m, 112H), 0.88-0.85 (m, 18H). ¹³C NMR (100 MHz, CDCl₃): δ ppm=167.02, 152.76, 148.45, 145.93, 139.98, 133.99, 131.14, 127.78, 124.09, 123.11, 122.72, 116.27, 92.92, 74.67, 72.19, 69.90, 48.31, 43.53, 37.43, 32.31, 31.89, 30.35, 30.09, 26.60, 23.11, 14.54. HR-APCI-MS: m/z=1901.9589, calculated exact mass: 1901.9568, error: 1.17 ppm.

Stille Cross-coupling Polymerization.

1,4-Bis(5-(8-bromo-3,3-bis((dodecyloxy)methyl)-3,4-dihydro-2H-thieno[3,4-b][1,4] dioxepin-6-yl)-7-(2-octyldodecyl)-6H-pyrrolo[3,4-g]-phthalazine-6,8(7H)-dione (13) (300 mg, 0.158 mmol) and bis(trimethylstannyl) compounds 14 (0.158 mmol) were dissolved in 10 ml of toluene. The mixture was stirred and purged with argon for about 15 min, and then Pd(PPh₃)₄ (6% equiv., 0.00948 mmol, 11 mg) was added. The flask was purged with argon for another 15 min before the mixture was heated to 110° C. for 2 days. Excess solvent was removed under reduced pressure and the polymer was precipitated with methanol. The suspension was filtered to give the crude product, which was then purified by Soxhlet extraction with acetone and chloroform. The chloroform fraction was evaporated to dryness to afford the target polymer materials 15.

The bis(trimethylstannyl) compounds 14 were as follows:

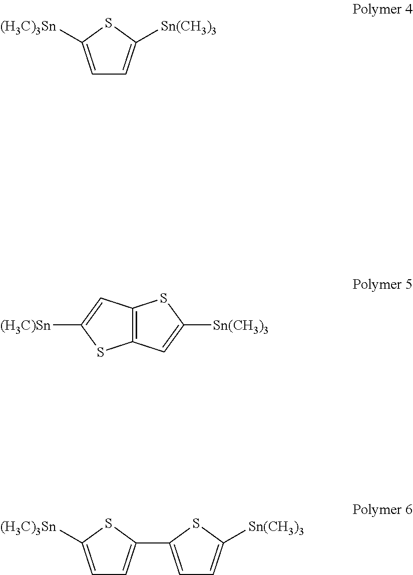

### EXAMPLE 9

### Characterization of Polymer 4, Polymer 5 and Polymer 6

¹H NMR Characterization

Polymer 4. Yield 74%. ¹H NMR (main signals) (CDCl₃): δ 9.09 (br), 7.92 (br), 4.31 (br, m), 3.72-3.45 (br, m), 1.68-1.58 (br, m), 1.24 (m), 0.87 (m).

Polymer 5. Yield 72%. NMR (main signals) (CDCl₃): δ 8.99 (br), 7.65 (br), 4.33 (br), 3.62-3.48 (br, m), 1.24 (br, m), 0.86 (br, m).

Polymer 6. Yield 55%. ¹H NMR (main signals) (CDCl₃): δ 9.08 (br), 7.82 (br), 6.97 (br), 4.31 (br), 3.70-3.48 (br, m), 1.79-1.68 (br), 1.24 (br), 0.86 (br).

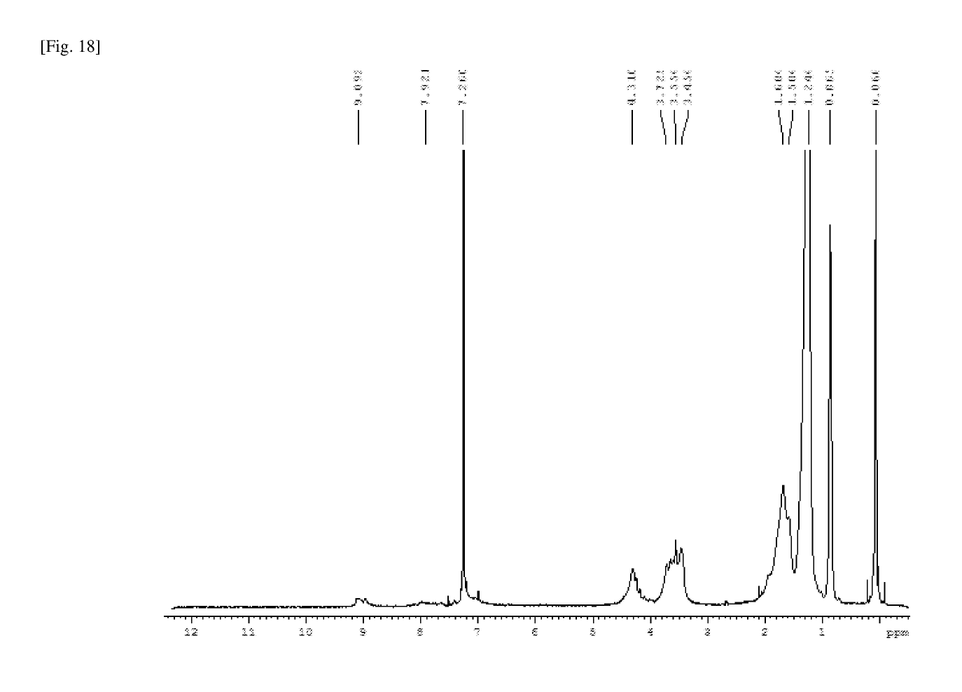

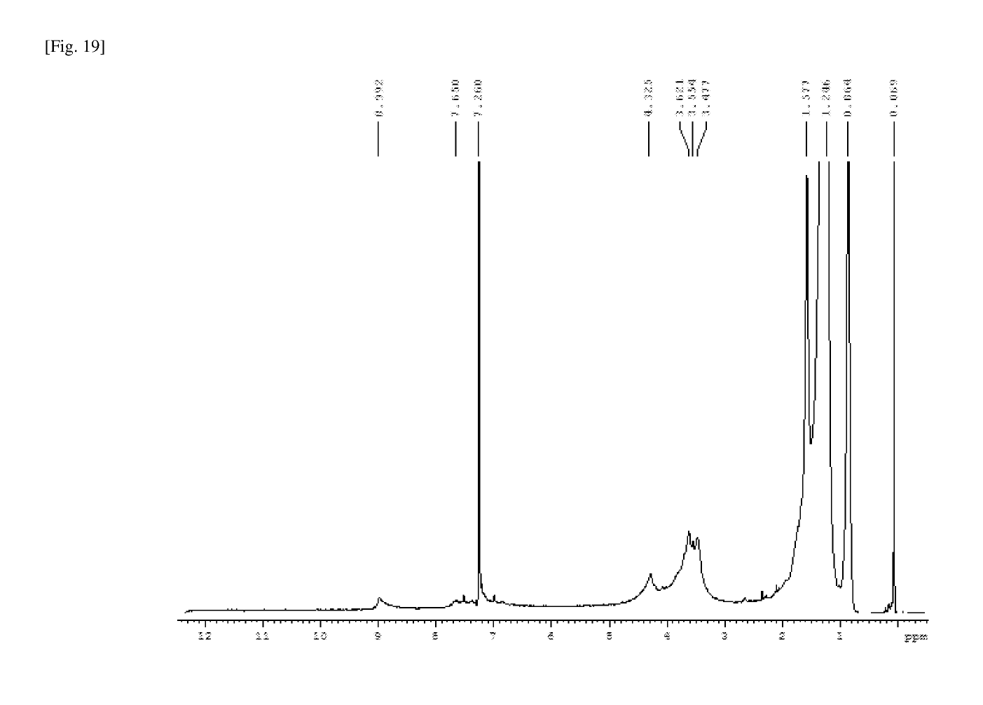

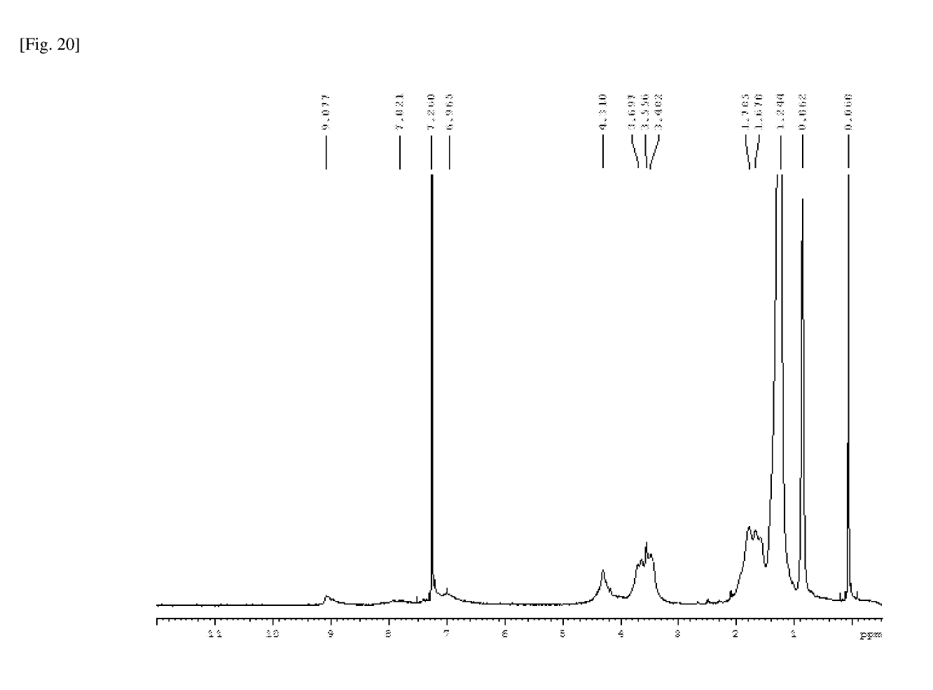

The NMR spectra of these Polymer 4, Polymer 5 and Polymer 6 are provided in FIG. 18, FIG. 19 and FIG. 20, respectively.

### EXAMPLE 10

### Manufacture of Devices Using Polymer 4, Polymer 5 and Polymer 6

Prior to use, ITO/glass substrates were cleaned by successive ultrasonication in acetone, isopropyl alcohol and distilled water, and blown dry with N₂. Polymer solutions of polymer 4, polymer 5 and polymer 6 were prepared at a concentration of 15 mg/mL in 6:4 (v/v) chloroform:chlorobenzene mixed solvent. Warm polymer solution (50° C.) was filtered and spin-coated onto the heated ITO substrates (50° C.) at 1200 rpm for 60 seconds to yield film thickness of around 100 nm. For drop-cast films, the polymer solutions are dispensed, spread onto the ITO substrates and left to dry. Film thickness of approximately 150-200 nm was obtained. Excessive polymer edges were removed by swabbing with chloroform using a cotton bud to obtain an active area of 2×2 cm². On a second piece of ITO substrate, an area of 2×2 cm² was blocked out using parafilm. The total thickness of the parafilm spacer and barrier was kept constant at 0.01″. 250 μL of the gel electrolyte (0.512 g of lithium perchlorate and 2.8 g of poly(methyl methacrylate) (MW=120,000 g/mol) in 6.65 ml of propylene carbonate and 28 ml of dry acetonitrile) was pipetted within the 2×2 cm² area. The device was fabricated by assembling the two ITO/glass substrates together with the polymer film and gel electrolyte in contact.

### EXAMPLE 11

### Characterization of Devices Manufactured Using Polymer 4, Polymer 5 and Polymer 6

Electron Capture (EC) Electrospectroscopy

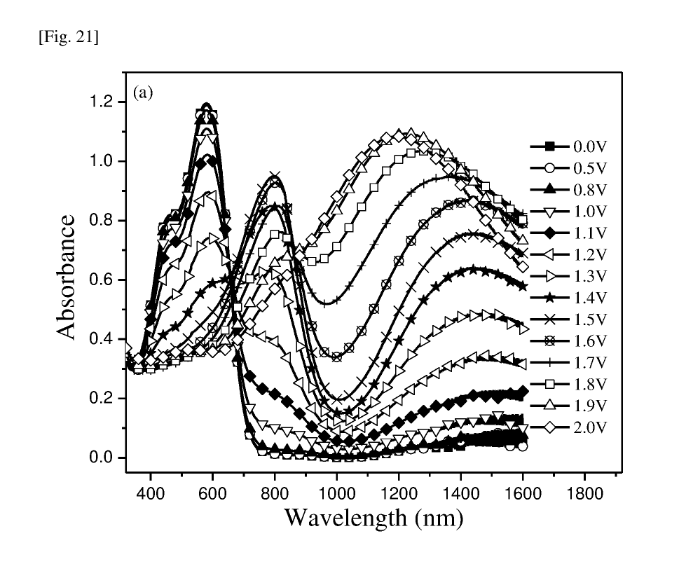

The electron capture (EC) electrospectroscopy of the polymer 5 in a EC device is depicted in FIG. 21.

FIG. 21 describes the UV-vis-NIR absorption behavior of polymer P5 electrochromic device upon oxidative voltage bias. When no voltage is applied on the polymer film, the polymer mainly absorbs around 500 nm and the neutral polymer film has a dark purple color. When an intermediate voltage is applied to the thin film, e.g., 1.5 V, the partially oxidized polymer film mainly absorbs around 800 nm. At this voltage, the thin film color is changed to light brown. When even higher voltage is applied at 2.0 V, the absorption of the polymer in the visible light region (400-700 nm) is depleted. The polymer film at 2.0 V mainly absorbs in the NIR region with a broad peak at 1200 nm. The polymer thin film then appears to be light cyan-colored. This indicates that the polymer can be used as a useful material for color-switching smart window applications.

The electrochromic performance of the polymer devices of Polymer 4, Polymer 5 and Polymer 6 are summarized in the following Table 8:

TABLE 8
Electrochromic performance of Devices
Comprising Polymers 4, 5 and 6

| | Optical contrast | | Bleaching | | Coloration | | CE | |
|---|---|---|---|---|---|---|---|---|
| | (%) | | time (s) | | time (s) | | (cm²/C.) | |
| | | 1400 | | 1400 | | 1400 | | 1400 |
| | λmax | nm | λmax | nm | λmax | nm | λmax | nm |
| Polymer 4 | 11 | 58 | 23.2 | 17.6 | 4.0 | 23.6 | 205 | 379 |
| Polymer 5 | 34 | 71 | 23.0 | 2.59 | 1.8 | 17.7 | 471 | 651 |
| Polymer 6 | 14 | 62 | 25.3 | 17.9 | 4.5 | 23.6 | 274 | 366 |

The optical contrast refers to how much the color of the film has changed upon an applied voltage. Polymers 4, 5 and 6 showed reasonably good optical contrast in the visible region and good contrast in the NIR region. The bleaching time and coloration time refer to how fast the device changes its color. Polymers 4, 5 and 6 showed good coloration time below 5 seconds. This means that upon an applied voltage, the thin film device will reach their full optical contrast in less than 5 seconds. The coloration efficiency describes how much electricity is needed to power the polymer device. The coloration efficiency for this batch of polymers is generally good with values greater than 200 cm²/C.

### EXAMPLE 12

### Time-Dependent Density Functional Theory Calculation

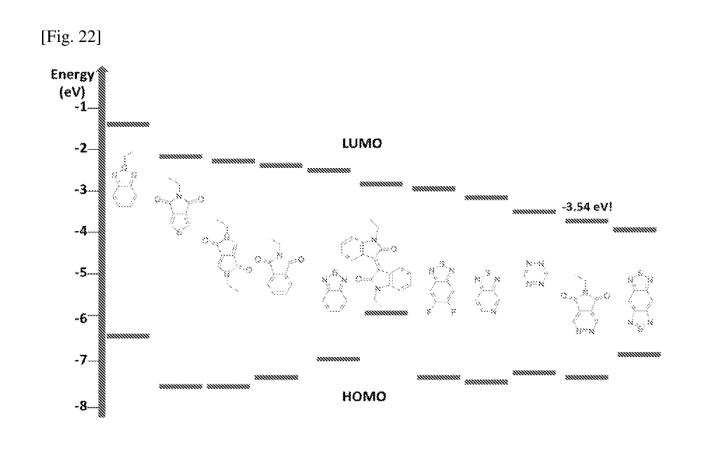

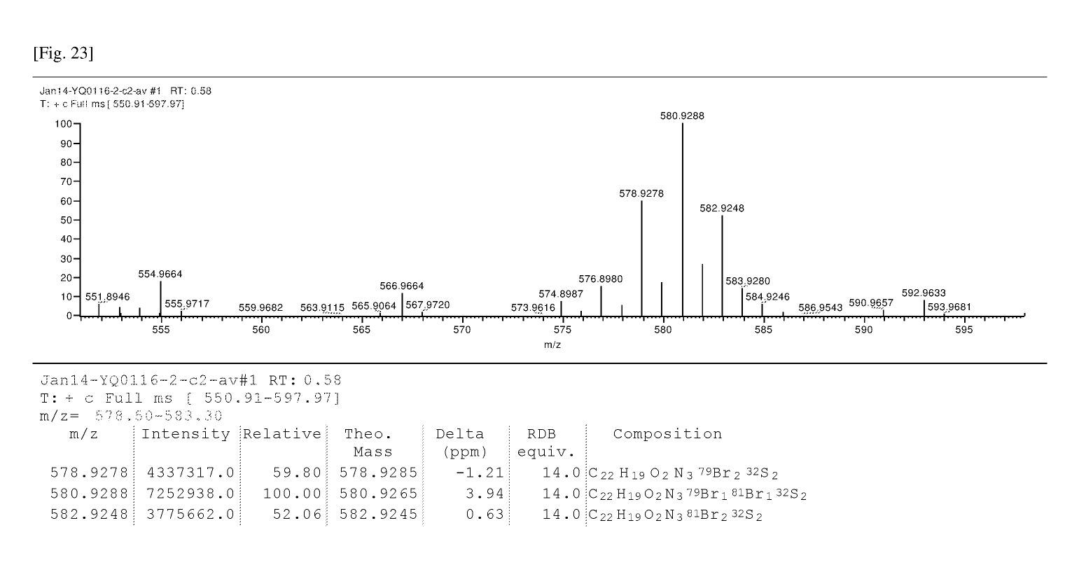

Time-dependent density-functional theory calculation is carried out to compare the magnitude of electron deficiency of the imide-fused pyridazines with current commonly used electron deficient acceptors. The results shown in FIG. 22 revealed that all widely used monomers, such as dikeptopyrrolopyrrole (DPP), benzotriazole (BT), iso-indigo, tetrazine, etc have lower electron-deficiency than imide-fused pyridazines synthesized according to the present disclosure. There is only one known electron-deficient acceptor benzo(bis)thiadiazole (BBT) which has higher electron deficiency than the imide-fused pyridazines of the present disclosure. However, BBT has no suitable position to undergo further functionalization, and hence BBT-based polymers typically have poor solubility in solvents. In contrast, imide-fused pyridazine can be conveniently functionalized with flexible alkyl chains to enhance its process ability, making it more promising to integrate with other donors to prepare more processable polymers.

### EXAMPLE 13

Monomer 1:

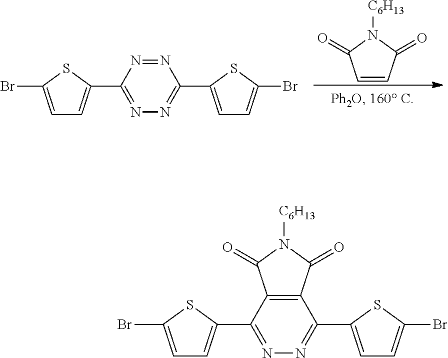

1,4-Bis(5-bromothiophen-2-yl)-6-hexyl-5H-pyrrolo[3,4-d]-pyridazine-5,7(6H)-dione

To a dry round bottom flask was added 3,6-bis(5-bromothiophen-2-yl)-1,2,4,5-tetrazine (300 mg, 0.743 mmol), 1-(2-hexyl)-1H-pyrrole-2,5-dione (135 mg, 0.743 mmol) and diphenyl ether (10 ml). The mixture was purged with argon for 15 min and then heated to 160° C. for 16 h. After cooling to room temperature, the mixture was directly subject to silica gel column chromatography. Hexane was used first to flush away the diphenyl ether. The target compound was then collected as yellow powder using mixture solvent (Hex:CHCl₃=1:1) then CHCl₃ as eluent (230 mg, 56%). ¹H NMR (400 MHz, CDCl₃): δ ppm=8.62-8.61 (d, J=4 Hz, 2H), 7.20-7.18 (d, J=3.6 Hz, 2H), 3.79-3.76 (t, J=7.6 Hz, 2H), 1.72-1.67 (m, 2H), 1.33 (m, 6H), 0.91-0.87 (t, J=6.4 Hz, 3H). ¹³C NMR (100 MHz, CDCl₃): δ ppm=167.47, 148.32, 139.74, 134.33, 132.09, 122.57, 121.47, 39.64, 31.66, 28.58, 26.92, 22.87, 14.40. HR-EI-MS: m/z=552.9136, calculated exact mass: 552.9123, error: 2.28 ppm.

Monomer 3:

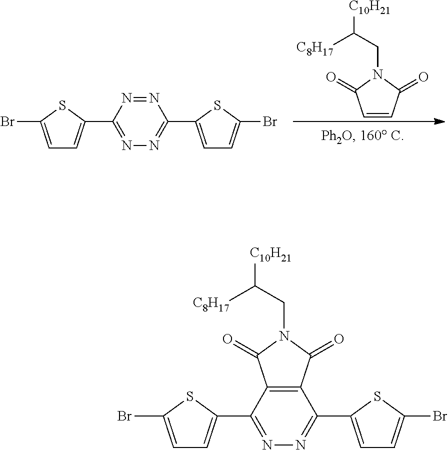

1,4-Bis(5-bromothiophen-2-yl)-6-(2-octyldodecyl)-5H-pyrrolo[3,4-d]pyridazine-5,7(6H)-dione:

To a dry round bottom flask was added 3,6-bis(5-bromothiophen-2-yl)-1,2,4,5-tetrazine (3 g, 7.43 mmol), 1-(2-octyldodecyl)-1H-pyrrole-2,5-dione (2.8 g, 7.43 mmol) and diphenyl ether (20 ml). The mixture was purged with argon for 15 min and then heated to 160° C. for 16 h. After cooling to room temperature, the mixture was directly subject to silica gel column chromatography. Hexane was used first to flush away the diphenyl ether. The target compound was then collected as yellow powder using mixture solvent (Hex:CHCl₃=1:1) then CHCl₃ as eluent (3.42 g, 62%). ¹H NMR (400 MHz, CDCl₃): δ ppm=8.63-8.62 (d, J=4 Hz, 2H), 7.19-7.18 (d, J=4 Hz, 2H), 3.67-3.65 (d, J=7.2 Hz, 2H), 1.90 (m, 1H), 1.25-1.24 (m, 32H), 0.89-0.85 (m, 6H). ¹³C NMR (100 MHz, CDCl₃): δ ppm=167.72, 148.30, 139.78, 134.38, 132.09, 122.47, 121.47, 43.81, 37.37, 32.31, 32.27, 31.85, 30.31, 30.02, 29.97, 29.93, 29.75, 29.68, 26.62, 23.08, 14.52. HR-EI-MS: m/z=750.1408, calculated exact mass: 750.1393, error: −2.1 ppm.

Monomer 4:

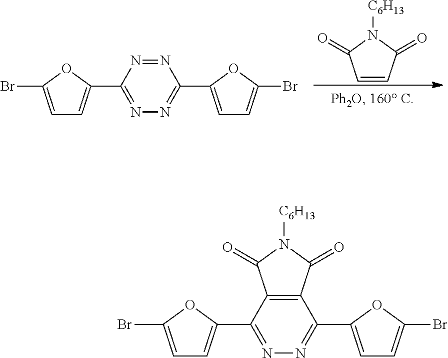

1,4-Bis(5-bromofuran-2-yl)-6-hexyl-5H-pyrrolo[3,4-d]pyridazine-5,7(6H)-dione:

To a dry round bottom flask was added 3,6-bis(5-bromofuran-2-yl)-1,2,4,5-tetrazine (300 mg, 0.81 mmol), 1-(2-hexyl)-1H-pyrrole-2,5-dione (146 mg, 0.81 mmol) and diphenyl ether (10 ml). The mixture was purged with argon for 15 min and then heated to 160° C. for 16 h. After cooling to room temperature, the mixture was directly subject to silica gel column chromatography. Hexane was used first to flush away the diphenyl ether. The target compound was then collected as yellow powder using mixture solvent (Hex:CHCl₃=1:1) then CHCl₃ as eluent (310 mg, 73%). ¹H NMR (400 MHz, CDCl₃): δ ppm=8.03-8.02 (d, J=3.6 Hz, 2H), 6.62-6.61 (d, J=4 Hz, 2H), 3.77-3.73 (t, J=7.6 Hz, 2H), 1.70-1.65 (m, 2H), 1.32 (m, 6H), 0.89-0.86 (t, J=6.4 Hz, 3H). ¹³C NMR (100 MHz, CDCl₃): δ ppm=167.72, 148.30, 139.78, 134.38, 132.09, 122.47, 121.47, 43.81, 37.37, 32.31, 32.27, 31.85, 30.31, 30.02, 29.97, 29.93, 29.75, 29.68, 26.62, 23.08, 14.52. HR-EI-MS: m/z=520.9586, calculated exact mass: 520.9580, error: 1.04 ppm.

Monomer 6:

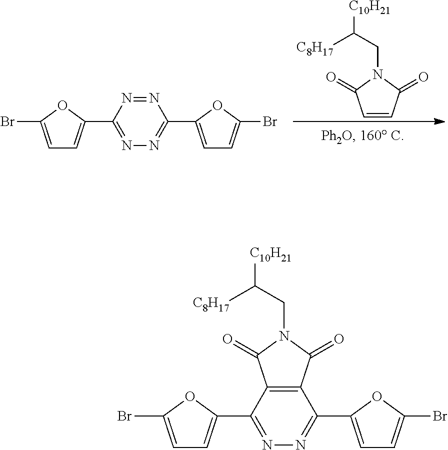

1,4-Bis(5-bromofuran-2-yl)-6-(2-octyldodecyl)-5H-pyrrolo[3,4-d]pyridazine-5,7(6H)-dione:

To a dry round bottom flask was added 3,6-bis(5-bromofuran-2-yl)-1,2,4,5-tetrazine (1.48 g, 4 mmol), 1-(2-octyldodecyl)-1H-pyrrole-2,5-dione (4 mmol, 1.51 g) and diphenyl ether (15 ml). The mixture was degassed by purging with argon and then heated to 160° C. for 16 h. After cooling to room temperature, the mixture was directly subject to silica gel column chromatography. Hexane was used first to flush away the diphenyl ether. The target compound was then collected as yellow powder using mixture solvent (Hex:CHCl₃=1:1) then CHCl₃ as eluent (1.86 g, 65%). ¹H NMR (400 MHz, CDCl₃): δ ppm=8.06 (d, 2H, J=3.6 Hz), 6.64-6.63 (d, 2H, J=3.6 Hz), 3.66-3.64 (d, 2H, J=7.6 Hz), 1.57 (m, 1H), 1.24 (m, 32H), 0.89-0.85 (m, 6H). ¹³C NMR (100 MHz, CDCl₃): δ ppm=166.89, 149.69, 143.31, 128.45, 122.25, 121.37, 115.17, 43.77, 37.29, 32.30, 31.81, 30.30, 30.01, 29.73, 29.67, 26.59, 23.07, 14.51. HR-EI-MS: m/z=719.1476, calculated exact mass: 719.1756, error: −1.41 ppm.

Monomer 10—Step 1:

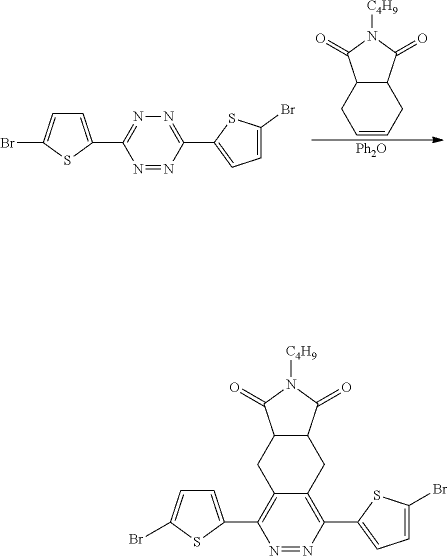

1,4-Bis(5-bromothiophen-2-yl)-7butyl-5,5a,8a,9-tetrahydro-6H-pyrrolo[3,4-g]phthalazine-6,8(7H)-dione:

To a dry round bottom flask was added 3,6-bis(5-bromothiophen-2-yl)-1,2,4,5-tetrazine (400 mg, 0.1 mmol), 2-butyl-3a,4,7,7a-tetrahydro-1H-isoindole 1,3(2H)-dione (207 mg, 0.1 mmol) and diphenyl ether (10 ml). The mixture was purged with argon for 15 min and then heated to 160° C. for 16h. After cooling to room temperature, the mixture was directly subject to silica gel column chromatography. Hexane was used first to flush away the diphenyl ether. The target compound was then collected as yellow powder using mixture solvent (Hex:CHCl₃=1:1) then CHCl₃ as eluent (410 mg, 71%). ¹H NMR (400 MHz, CDCl₃): δ ppm=7.32-7.31 (d, J=4 Hz, 2H), 7.16-7.15 (d, J=4 Hz, 2H), 3.83-3.79 (m, 2H), 3.44-3.42 (m, 2H), 3.38-3.35 (t, J=6.8 Hz, 2H), 2.94-2.90 (m, 2H), 1.27-1.22 (m, 2H), 0.74-0.70 (t, J=6.8 Hz, 3H). ¹³C NMR (100 MHz, CDCl₃): δ ppm=178.33, 151.97, 140.45, 133.24, 131.26, 129.88, 117.50, 39.39, 38.88, 29.69, 25.21, 19.85, 13.80. HR-EI-MS: m/z=580.9288, calculated exact mass: 580.9265, error: 3.94 ppm.

The HR-EI MS spectrum for this intermediate is shown further below as FIG. 22.

Monomer 10—Step 2:

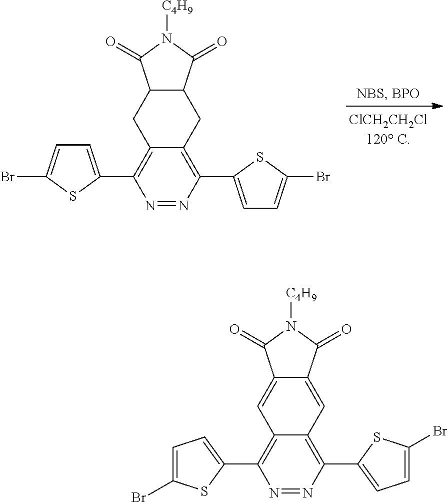

1,4-Bis(5-bromothiophen-2-yl)-7-butyl-6H-pyrrolo[3,4-g]phthalazine-6,8(7H)-dione:

To a dry round bottom flask was added 1,4-bis(5-bromothiophen-2-yl)-7-butyl-5,5a,8a,9-tetrahydro-6H-pyrrolo[3,4-g]phthalazine-6,8(7H)-dione (200 mg, 0.345 mmol), NBS (2.2 equiv., 0.76 mmol, 135 mg), BPO (0.1 equiv. 0.0345 mol, 10 mg) and dichloroethane (10ml). The mixture was purged with argon for 10 min and then heated to 120° C. for 2 days. After cooling to room temperature, the mixture was directly subject to silica gel column chromatography. Chloroform was used as the eluent and the target product was obtained as a yellow solid. (135 mg, 68%) ¹H NMR (400 MHz, CDCl₃): δ ppm=.8.92 (s, 2H), 7.52-7.51 (d, J=4 Hz, 2H), 7.29-7.28 (d, J=3.6 Hz, 2H), 3.83-3.80 (t, J=7.2 Hz, 2H), 1.76-1.68 (m, 2H), 1.45-1.37 (m. 2H), 0.99-0.95 (t, J=7.6 Hz, 3H) ¹³C NMR (100 MHz, CDCl₃): δ ppm=166.52, 152.63, 139.69, 134.39, 131.69, 131.61, 127.66, 122.35, 119.26, 39.14, 30.82, 20.49, 14.00. HR-EI-MS: m/z=572.8961, calculated exact mass: 576.8952, error: 1.65 ppm.

### EXAMPLE 14

### Further Experimental Examples Relating to the Polymer Syntheses

Polymer 7:

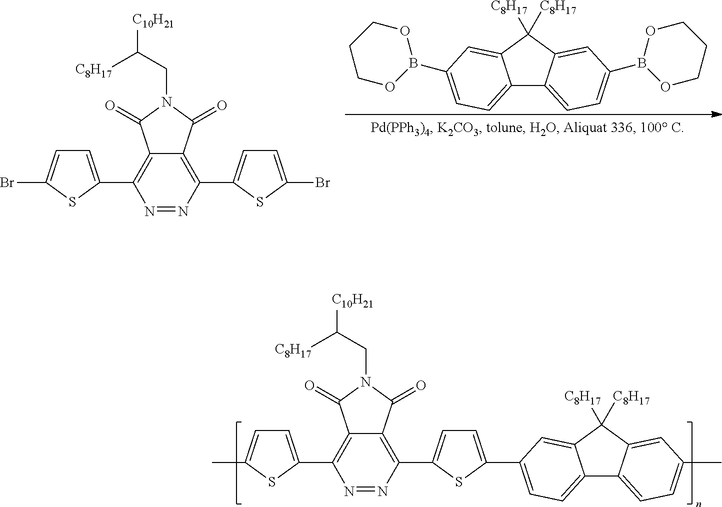

To a dry round bottom flask was added 1,4-bis(5-bromothiophen-2-yl)-6-(2-octyldodecyl)-5H-pyrrolo[3,4-d]pyridazine-5,7(6H)-dione (225 mg, 0.3 mmol), 2,2′-(9,9-dioctyl-9H-fluorene-2,7-diyl)bis(1,3,2-dioxaborinane) (168 mg, 0.3 mmol), Pd(PPh₃)₄ (6 mol %, 0.018 mmol, 21 mg), K₂CO₃ (2 mmol, 276 mg), H₂O (1 ml), toluene (10 ml) and Aliquat 336 (catalytic amount). The mixture was purged with argon for 30 min and then heated to 100° C. for 48 h. After cooling to room temperature, chloroform was added to dilute the solution and then extraction with water was done for three times. The organic layer was collected and dried by rotary evaporation. The residue was dissolved in minimal amount of chloroform and precipitated into methanol solution. The red precipitate was collected via suction filtration. The polymer was purified by Soxhlet extraction with hexane for 20 h, followed by acetone for 20 h and finally washed down by chloroform. The chloroform part was concentrated and dropped into methanol solution with rigorous stirring. The precipitate was collected via suction filtration, thoroughly washed with methanol and dried under vacuum. (250 mg, 85%)

Polymer 8:

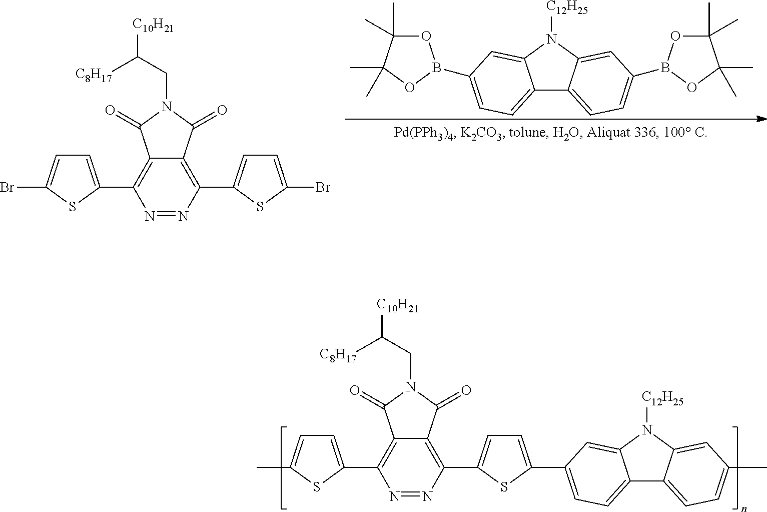

To a dry round bottom flask was added 1,4-bis(5-bromothiophen-2-yl)-6-(2-octyldodecyl)-5H-pyrrolo[3,4-d]pyridazine-5,7(6H)-dione (225 mg, 0.3 mmol), 9-(2-dodecyl)-2,7-bis(4,4,5,5-tetramethyl-1,3,2-dioxaborolan-2-yl)-9H-carbazole (210 mg, 0.3 mmol), Pd(PPh₃)₄ (6 mol %, 0.018 mmol, 21 mg), K₂CO₃ (2 mmol, 276 mg), H₂O (1 ml), toluene (10 ml) and Aliquat 336 (catalytic amount). The mixture was purged with argon for 30 min and then heated to 100° C. for 48 h. After cooling to room temperature, chloroform was added to dilute the solution and then extraction with water was done for three times. The organic layer was collected and dried by rotary evaporation. The residue was dissolved in minimal amount of chloroform and precipitated into methanol solution. The purple precipitate was collected via suction filtration. The polymer was purified by Soxhlet extraction with hexane for 20 h, followed by acetone for 20 h and finally washed down by chloroform. The chloroform part was concentrated and dropped into methanol solution with rigorous stirring. The precipitate was collected via suction filtration, thoroughly washed with methanol and dried under vacuum. (250 mg, 85%)

Polymer 9:

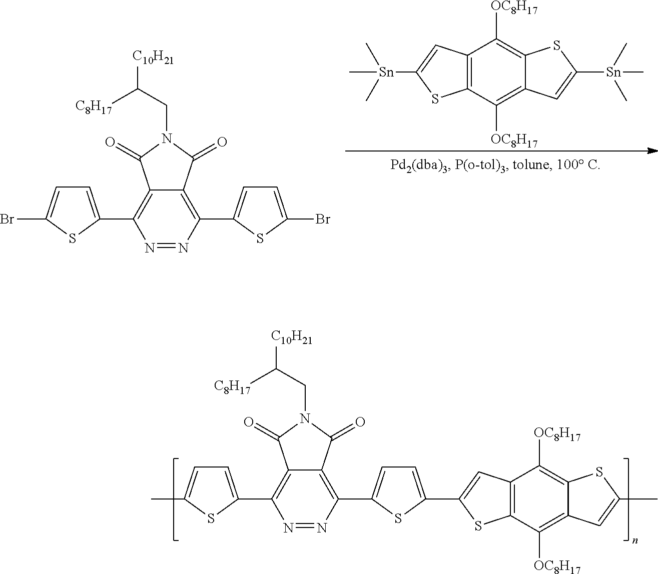

To a dry round bottom flask was added 1,4-bis(5-bromothiophen-2-yl)-6-(2-octyldodecyl)-5H-pyrrolo[3,4-d]pyridazine-5,7(6H)-dione (225 mg, 0.3 mmol), (4,8-bis(octyloxy)benzo[1,2-b:4,5-b′]dithiophene-2,6-diyl)bis(trimethylstannane) (232 mg, 0.3 mmol), Pd₂(dba)₃ (3 mol %, 0.009 mmol, 8.5 mg), P(o-tol)₃ (12 mol %, 0.036 mmol, 11 mg) and toluene (10 ml). The mixture was purged with argon for 30 min and then heated to 100° C. for 16 h. After cooling to room temperature, chloroform was added to dilute the solution and then extraction with water was done for three times. The organic layer was collected and dried by rotary evaporation. The residue was dissolved in minimal amount of chloroform and precipitated into methanol solution. The precipitate was collected via suction filtration. The polymer was purified by Soxhlet extraction with hexane for 20 h, followed by acetone for 20 h and finally washed down by chloroform. The chloroform part was concentrated and dropped into methanol solution with rigorous stirring. The precipitate was collected via suction filtration, thoroughly washed with methanol and dried under vacuum. (230 mg, 74%)

Polymer 10:

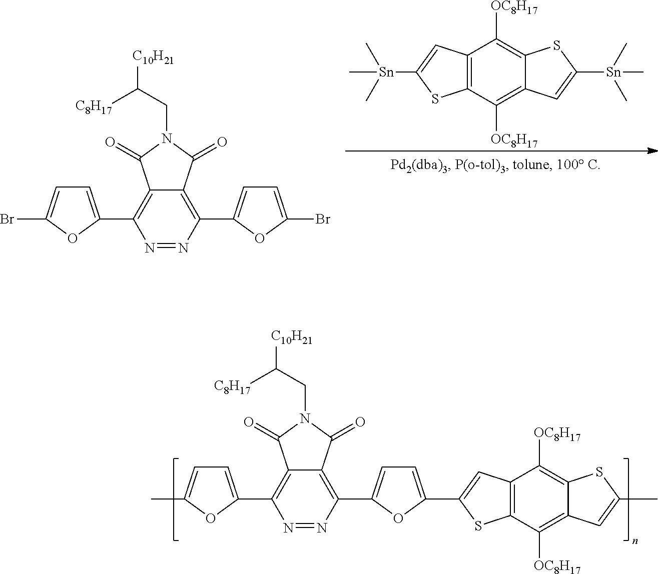

To a dry round bottom flask was added 1,4-bis(5-bromofuran-2-yl)-6-(2-octyldodecyl)-5H-pyrrolo[3,4-d]pyridazine-5,7(6H)-dione (216 mg, 0.3 mmol), (4,8-bis(octyloxy)benzo[1,2-b:4,5-b′]dithiophene-2,6-diyl)bis(trimethylstannane) (232 mg, 0.3 mmol), Pd₂(dba)₃ (3 mol %, 0.009 mmol, 8.5 mg), P(o-tol)₃ (12 mol %, 0.036 mmol, 11 mg) and toluene (10 ml). The mixture was purged with argon for 30 min and then heated to 100° C. for 16 h. After cooling to room temperature, chloroform was added to dilute the solution and then extraction with water was done for three times. The organic layer was collected and dried by rotary evaporation. The residue was dissolved in minimal amount of chloroform and precipitated into methanol solution. The precipitate was collected via suction filtration. The polymer was purified by Soxhlet extraction with hexane for 20 h, followed by acetone for 20 h and finally washed down by chloroform. The chloroform part was concentrated and dropped into methanol solution with rigorous stirring. The precipitate was collected via suction filtration, thoroughly washed with methanol and dried under vacuum. (215 mg, 72%)

Polymer 11:

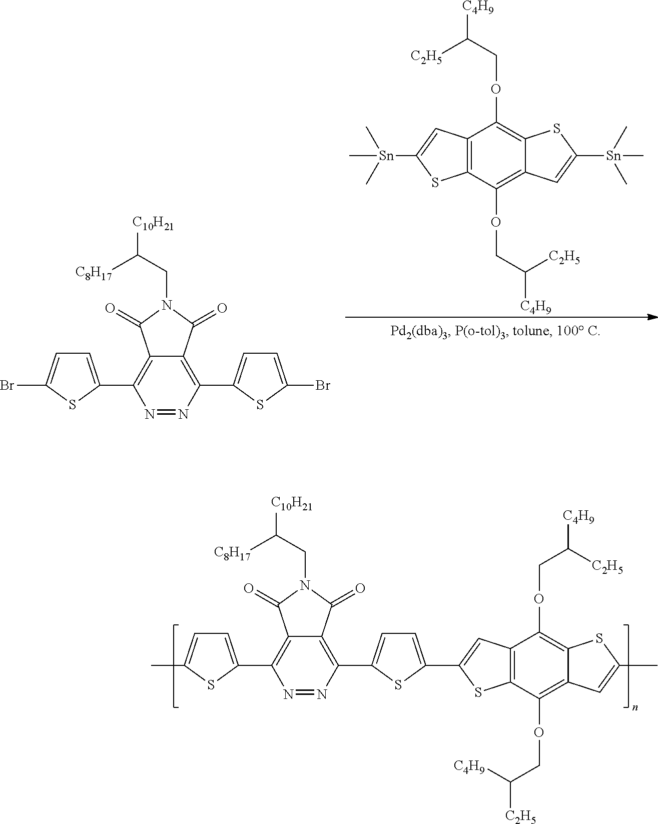

To a dry round bottom flask was added 1,4-bis(5-bromothiophen-2-yl)-6-(2-octyldodecyl)-5H-pyrrolo[3,4-d]pyridazine-5,7(6H)-dione (225 mg, 0.3 mmol), (4,8-bis((2-ethylhexyl)oxy)benzo[1,2-b:4,5-b′]dithiophene-2,6-diyl)bis(trimethylstannane) (232 mg, 0.3 mmol), Pd₂(dba)₃ (3 mol %, 0.009 mmol, 8.5 mg), P(o-tol)₃ (12 mol %, 0.036 mmol, 11 mg) and toluene (10 ml). The mixture was purged with argon for 30 min and then heated to 100° C. for 16 h. After cooling to room temperature, chloroform was added to dilute the solution and then extraction with water was done for three times. The organic layer was collected and dried by rotary evaporation. The residue was dissolved in minimal amount of chloroform and precipitated into methanol solution. The precipitate was collected via suction filtration. The polymer was purified by Soxhlet extraction with hexane for 20 h, followed by acetone for 20 h and finally washed down by chloroform. The chloroform part was concentrated and dropped into methanol solution with rigorous stirring. The precipitate was collected via suction filtration, thoroughly washed with methanol and dried under vacuum. (210 mg, 68%)

Polymer 12:

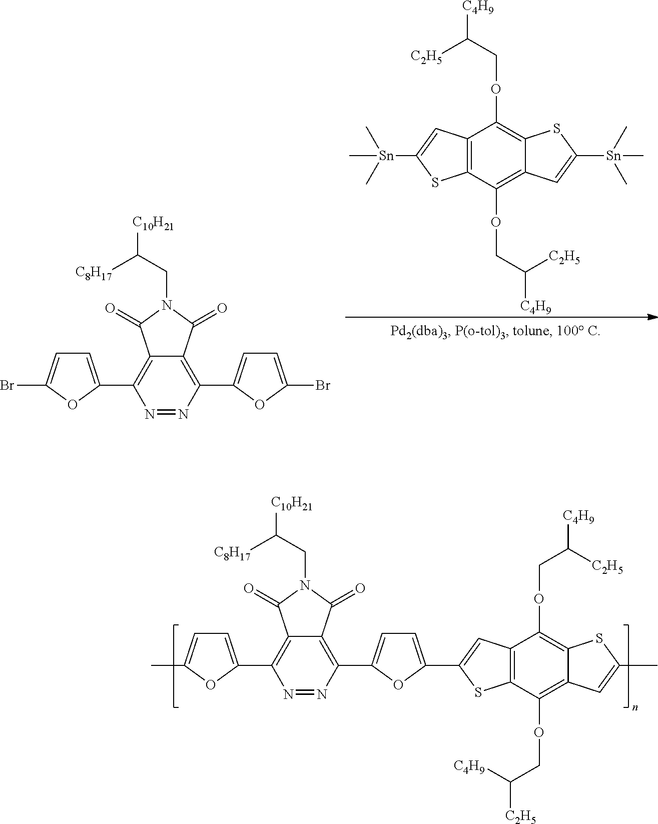

To a dry round bottom flask was added 1,4-bis(5-bromofuran-2-yl)-6-(2-octyldodecyl)-5H-pyrrolo[3,4-d]pyridazine-5,7(6H)-dione (216 mg, 0.3 mmol), (4,8-bis((2-ethylhexyl)oxy)benzo[1,2-b:4,5-b′]dithiophene-2,6-diyl)bis(trimethylstannane) (232 mg, 0.3 mmol), Pd₂(dba)₃ (3 mol %, 0.009 mmol, 8.5 mg), P(o-tol)₃ (12 mol %, 0.036 mmol, 11 mg) and toluene (10 ml). The mixture was purged with argon for 30 min and then heated to 100° C. for 16 h. After cooling to room temperature, chloroform was added to dilute the solution and then extraction with water was done for three times. The organic layer was collected and dried by rotary evaporation. The residue was dissolved in minimal amount of chloroform and precipitated into methanol solution. The precipitate was collected via suction filtration. The polymer was purified by Soxhlet extraction with hexane for 20 h, followed by acetone for 20 h and finally washed down by chloroform. The chloroform part was concentrated and dropped into methanol solution with rigorous stirring. The precipitate was collected via suction filtration, thoroughly washed with methanol and dried under vacuum. (200 mg, 67%)

### INDUSTRIAL APPLICABILITY

The compounds of Formula (VI), (VIIa) and (VIIb) as defined above may be useful in the manufacture of the compounds of Formula (I). The compound of Formula (I) may be useful in the manufacture of the compounds of Formula (IV). The compounds of Formula (IV) may be useful in the manufacture of semiconductor devices. The semiconductor devices manufactured using the compound of Formula (IV) as defined above may be used in a wide range of applications such as electrochromic materials, organic light-emitting diode, organic thin-film transistors and organic photovoltaic cells. As the NIR absorption properties can be reversibly switched by applying/removing a low voltage on the thin film, the materials may have applications in smart glass or smart window applications.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A compound having the following Formula (I); 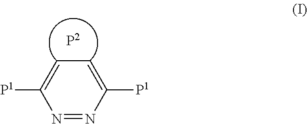
wherein P¹ has the following Formula (IIa) or (IIb); 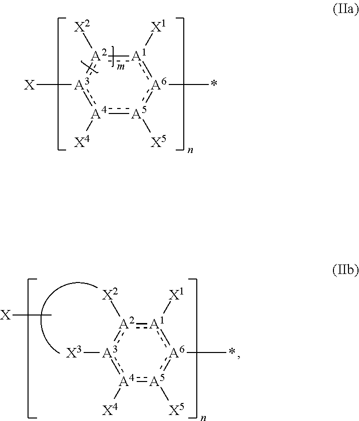
wherein [symbol]
is a single or double bond, as valency allows;
m is 0 or 1;
n is an integer selected from 1 to 4;
X, X¹, X², X³, X⁴ and X⁵ are independently absent or selected from the group consisting of a bond, H, halogen, CN, R¹, OR¹ or SR¹;
wherein R¹ is selected from the group consisting of optionally substituted alkyl, optionally substituted alkenyl, optionally substituted alkynyl, optionally substituted cycloalkyl, optionally substituted heterocycloalkyl, optionally substituted cycloalkenyl, optionally substituted heterocycloalkenyl, optionally substituted aryl, optionally substituted heteroaryl,
A¹ is selected from the group consisting of O, S, Se, Te, C and N
A², A³, A⁴ A⁵ and A⁶ are independently C or N; and
wherein any two substituents selected from the group consisting of X¹, X², X³, X⁴ and X⁵ may optionally be taken together to form an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl;
and wherein P² has the following formula (IIIa) or (IIIb); 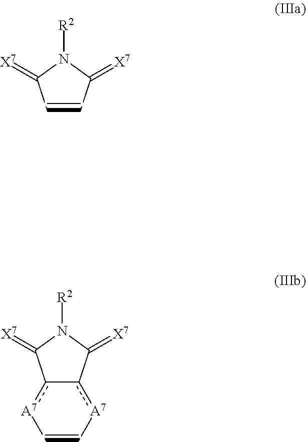
wherein
R² is an optionally substituted alkyl or optionally substituted aryl;
X⁷ is O or S;
A⁷ is CH₂ or N;
and [symbol] is where the compound of Formula (IIIa) or (IIIb) is fused to the compound of Formula (I).

2. A compound having repeating units of the following Formula (IV); 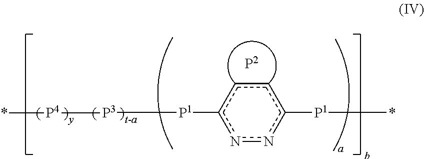
wherein P¹ has the following Formula (IIA) or (IIb); 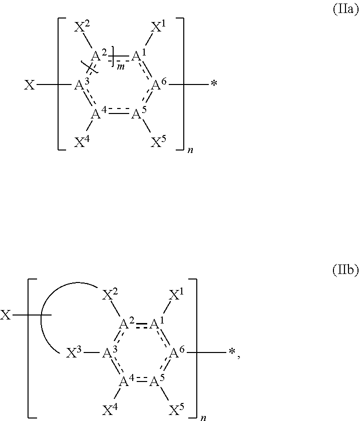
wherein [symbol]
is a single or double bond, as valency allows;
m is 0 or 1;
n is an integer selected from 1 to 4;
X¹, X², X³, X⁴ and X⁵ are independently absent or selected from the group consisting of a bond, H, halogen, CN, R¹, OR¹ or SR¹;
wherein R¹ is selected from the group consisting of optionally substituted alkyl, optionally substituted alkenyl, optionally substituted alkynyl, optionally substituted cycloalkyl, optionally substituted heterocycloalkyl, optionally substituted cycloalkenyl, optionally substituted heterocycloalkenyl, optionally substituted aryl, optionally substituted heteroaryl,
A¹ is selected from the group consisting of O, S, Se, Te, C and N
A², A³, A⁴, A⁵ and A⁶ are independently C or N; and
wherein any two substituents selected from the group consisting of X¹, X², X³, X⁴ and X⁵ may optionally be taken together to form an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl;
P² has the following formula (IIIa) or (IIIb); 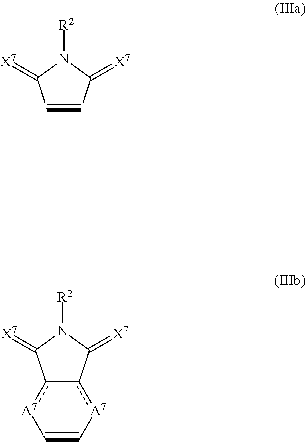
wherein
R² is an optionally substituted alkyl or optionally substituted aryl;
X⁷ is O or S;
A⁷ is CH, CH₂ or N;
and [symbol] is where the compound of Formula (IIIa) or (IIIb) is fused to the compound of Formula (IV);
P³ has the following Formula (Va), (Vb) or (Vc); 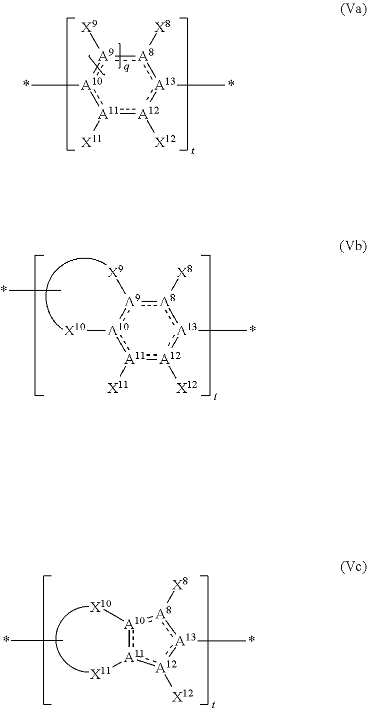
wherein
q is 0 or 1;
t is an integer selected from 1 to 4;
X⁸, X⁹, X¹¹ and X¹² are independently absent or selected from the group consisting of a bond, H, halogen, CN, R³, OR³ or SR³;
wherein R³ is selected from the group consisting of optionally substituted alkyl, optionally substituted alkenyl, optionally substituted alkynyl, optionally substituted cycloalkyl, optionally substituted heterocycloalkyl, optionally substituted cycloalkenyl, optionally substituted heterocycloalkenyl, optionally substituted aryl, optionally substituted heteroaryl,
A⁸ is selected from the group consisting of O, S, Se, Te, C and N
A⁹, A¹⁰, A¹¹, A¹² and A¹³ are independently C or N;
wherein any two substituents selected from the group consisting of X⁸, X⁹, X¹⁰, X¹¹ and X¹² may optionally be taken together to form an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl;
and P⁴ is absent, or is an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl.
a is any value from 0 to 1; and
b is an integer selected from 1 to 10,000; and
y is an integer selected from 0 to 5.

3. A compound having the following Formula (VI); 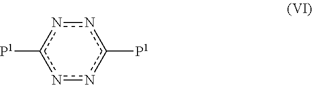
wherein P¹ has the following Formula (IIa) or (IIb); 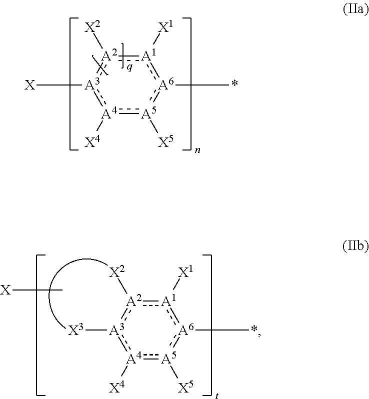
wherein [symbol]
is a single or double bond, as valency allows;
m is 0 or 1;
n is an integer selected from 1 to 4;
X, X¹, X², X³, X⁴ and X⁵ are independently absent or selected from the group consisting of a bond, H, halogen, CN, R¹, OR¹ and SR¹;
wherein R¹ is selected from the group consisting of optionally substituted alkyl, optionally substituted alkenyl, optionally substituted alkynyl, optionally substituted cycloalkyl, optionally substituted heterocycloalkyl, optionally substituted cycloalkenyl, optionally substituted heterocycloalkenyl, optionally substituted aryl, optionally substituted heteroaryl;
A¹ is selected from the group consisting of O, S, Se, Te, C and N;
A², A³, A⁴, A⁵ and A⁶ are independently C or N; and
wherein any two substituents selected from the group consisting of X¹, X², X³, X⁴ and X⁵ may optionally be taken together to form an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl.

4. A compound having the following formula (VIIa) or (VIIb); 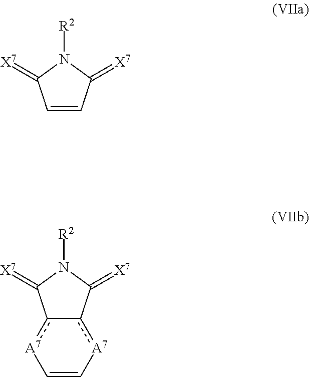
wherein [symbol]
is a single or double bond, as valency allows;
R² is an optionally substituted alkyl or optionally substituted aryl;
X⁷ is O or S; and
A⁷ is CH, CH₂ or N.

5. The compound according to claim 1, wherein halogen is selected from the group consisting of F, Cl, Br and I, the optionally substituted alkyl is an optionally substituted C₁-C₂₅ alkyl, the optionally substituted alkenyl is an optionally substituted C₂-C₂₅ alkenyl, the optionally substituted alkynyl is an optionally substituted C₂-C₂₅ alkynyl, the optionally substituted cycloalkyl is an optionally substituted C₃-C₁₂ cycloalkyl, the optionally substituted heterocycloalkyl is an optionally substituted C₃-C₁₂ heterocycloalkyl, the optionally substituted cycloalkenyl is an optionally substituted C₃-C₁₂ cycloalkenyl, the optionally substituted heterocycloalkenyl is an optionally substituted C₃-C₁₂ heterocycloalkenyl, the optionally substituted aryl is an optionally substituted C₃-C₁₈ aryl and the optionally substituted heteroaryl is an optionally substituted C₃-C₁₈ heteroaryl, wherein the optionally substituted heterocycloalkyl and optionally substituted heteroaryl are independently a 3-to 18-membered monocyclic, bicyclic, or polycyclic ring, X is selected from the group consisting of halogen, CN, R¹ and OR¹, wherein R¹ is optionally substituted alkyl, or X⁷ is O.

6-9. (canceled)

10. The compound according to claim 1, wherein P¹ is selected from the group consisting of optionally substituted furan, optionally substituted thiophene, optionally substituted selenophene, optionally substituted tellurophene, optionally substituted pyrrole, optionally substituted phenylene, optionally substituted aza-phenylene, optionally substituted arylene diimide, optionally substituted fluorene, optionally substituted cabazole, optionally substituted dibenzodiole, optionally substituted benzooxodiazole, optionally substituted benzotriazole, optionally substituted benzothiadiazole and any combination thereof or P¹ has the following Formula (IIa);

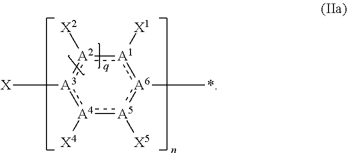

11. (canceled)

12. The compound according to claim 1, wherein m is 0, wherein A¹ is N, X¹ is H, A³, A⁴, A⁵ and A⁶ are C, or X⁴ and X⁵ are independently selected from the group consisting of H, R¹ and OR¹, wherein R¹ is optionally substituted alkyl, or wherein A¹ is S; X¹ is absent; A³, A⁴, A⁵ and A⁶ are C, X⁴ and X⁵ are H and X is Br, or A¹ is O; X¹ is absent; A³, A⁴, A⁵and A⁶ are C, X⁴ and X⁵ are H and X is Br.

13-19. (canceled)

20. The compound according to claim 1, wherein m is 1, wherein A³ and A⁴ are C, X¹, X², X⁴ and X⁶ are independently absent or selected form the group consisting of H, halogen, R¹ or OR¹, wherein R¹ is optionally substituted alkyl, A¹, A², A³, A⁴, A⁵ and A⁶ are C or X¹, X², X³, X⁴ and X⁶ are independently H or halogen.

21-25. (canceled)

26. The compound according to claim 1, wherein P² is selected from the group consisting of optionally substituted imide and optionally substituted phthalamide or R² is an optionally substituted alkyl, an optionally substituted linear alkyl or an optionally substituted branched alkyl, an optionally substituted C₁ to C₂₅ linear alkyl, or an optionally substituted C₁ to C₂₅ branched alkyl or wherein the optionally substituted branched alkyl has the following formula (XII) 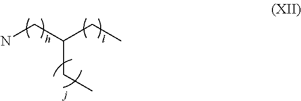
wherein
h is 0 or 1; and
i and j are independently integers selected from 1 to 13,
or wherein h is 1, or i is 7; and j is 9 or the optionally substituted alkyl is 2-octyldodecyl.

27-35. (canceled)

38. The compound according to claim 2, wherein P²has the following formula (IIIa): 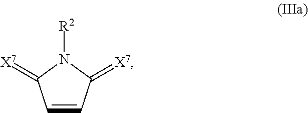
or P² has the following formula (IIIb): 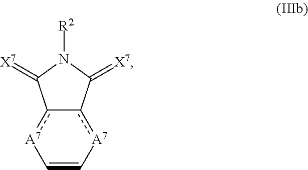
wherein A⁷ is CH₂ and P² has the following formula (IIIc): 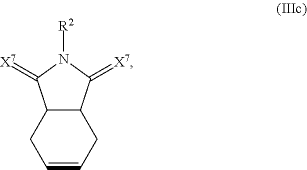
or A⁷ is CH and P² has the following Formula (IIId):

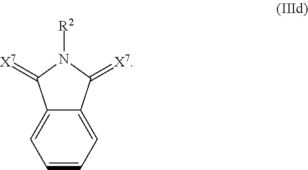

37-41. (canceled)

42. The compound according to claim 2, wherein P³ is selected from the group consisting of optionally substituted furan, optionally substituted thiophene, optionally substituted selenophene, optionally substituted pyrrole, optionally substituted phenyl, optionally substituted fluorenes, optionally substituted carbazoles, optionally substituted fused furans and optionally substituted fused thiophenes, or P³ has the following formula (Va);

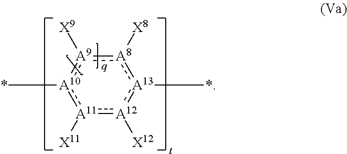

43. (canceled)

44. The compound according to claim 42, wherein q is 0, or A⁸ is N or X⁸ is H, A⁸ is S; X⁸ is absent; and A¹⁰, A¹¹, A¹² and A¹³ are C, X¹¹ and X¹² are taken together to form an optionally substituted 7-membered heterocycloalkyl or P³ has the following Formula (XVI);

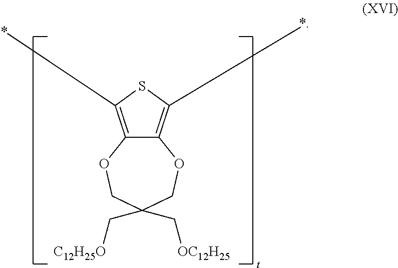

45-48. (canceled)

49. The compound according to claim 42, wherein q is 1, A¹⁰ and A¹³ are C, X⁸, X⁹, X¹¹ and X¹² are independently selected from the group consisting of H, halogen, R³, OR³, OR¹⁰ and SR¹⁰, R³ is optionally substituted alkyl, A⁸, A⁹, A¹⁰, A¹¹, A¹² and A¹³ are C, or R¹⁰ is an optionally substituted alkyl or has the following formula (Vb); 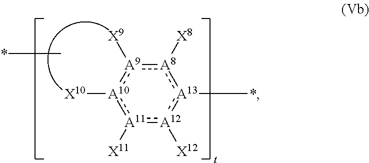
wherein A⁸, A⁹, A¹⁰, A¹¹, A¹² and A¹³ are C, X⁸, X¹¹ and X¹² are H, X⁹ and X¹⁰ are taken together to form an optionally substituted 5-membered heterocycloalkyl, wherein the optionally substituted 5-membered heterocycloalkyl is further fused with an optionally substituted 6membered aryl, or P³ has the following formula (XVII); 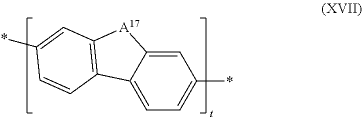
wherein A¹⁷ is selected from the group consisting of NH, CHR¹¹, C(R¹¹)₂, SiHR¹¹ and Si(R¹¹)₂; wherein R¹¹ is optionally substituted alkyl; wherein R¹¹ is C₈H₁₇or C₁₂H₂₅.

50-62. (canceled)

63. The compound according to claim 2, wherein P³ has the following Formula (Vc); 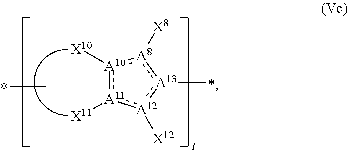
wherein A¹⁰, A¹¹, A¹² and A¹³ are C, X⁸ and X¹² are H, or X¹⁰ and X¹¹ are taken together to form an optionally substituted 5-membered heteroaryl or an optionally substituted 6-membered aryl, or P³ has the following Formula (XVIIIa) or (XVIIIb); 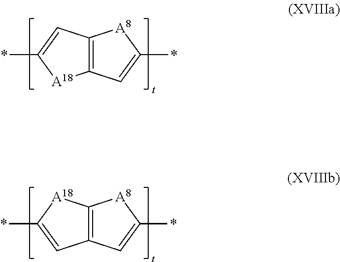
wherein A⁸ and A¹⁸ are independently selected from the group consisting of O, S and Se, or wherein the optionally substituted 6-membered aryl is further fused with an optionally substituted 5-membered heteroaryl, or P³ has the following Formula (XIX); 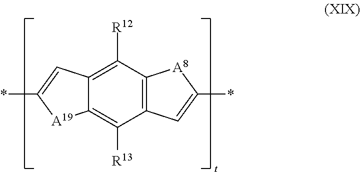
wherein A⁸ and A¹⁹ are independently selected from the group consisting of O, Se and Se; and R¹² and R¹³ are independently selected from R¹⁴, OR¹⁴, Sr¹⁴, wherein R¹⁴ is optionally substituted alkyl, optionally substituted aryl, C₈H₁₇ or 2-ethylheptyl.

64-73. (canceled)

74. The compound according to claim 2, wherein a is 0.5, 0.3 or 0.1 or b is an integer selected from 1 to 50, or P⁴ is selected from the group consisting of optionally substituted furan, optionally substituted thiophene, optionally substituted selenophene, optionally substituted pyrrole, optionally substituted phenyl, optionally substituted fluorenes, optionally substituted carbazoles, optionally substituted fused furans and optionally substituted fused thiophenes, an optionally substituted thiophene or an optionally substituted fused thiophene.

75-74. (canceled)

78. The compound according to claim 2, wherein y is 1, having the following formula (XX), (XXI), (XXII): 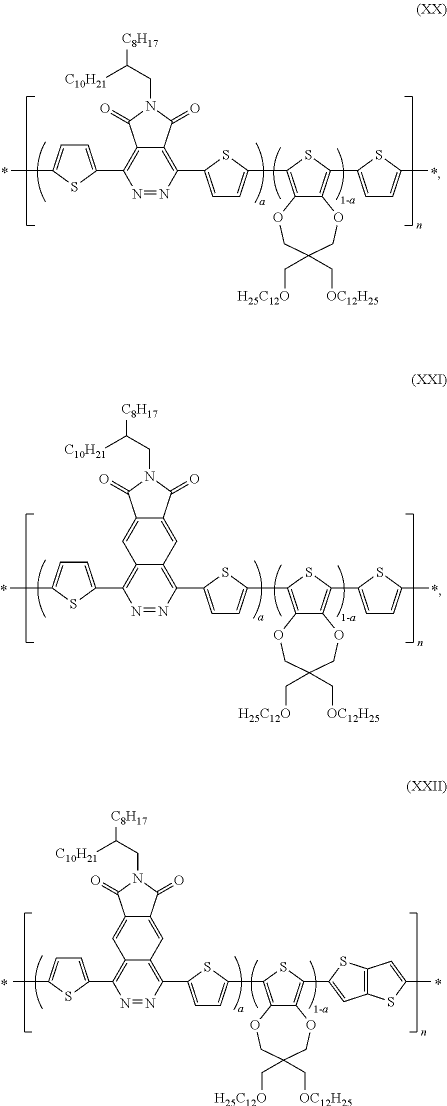
or wherein y is 2, having the following formula (XXIII),

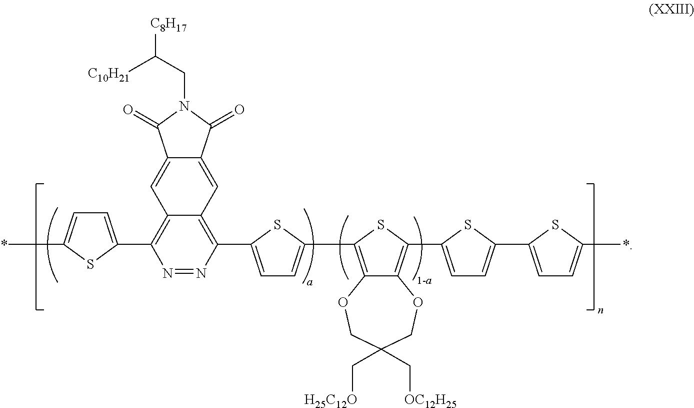

79-81. (canceled)

82. The compound according to claim 4, having the following Formula (XXIVa) or (XXIVb),

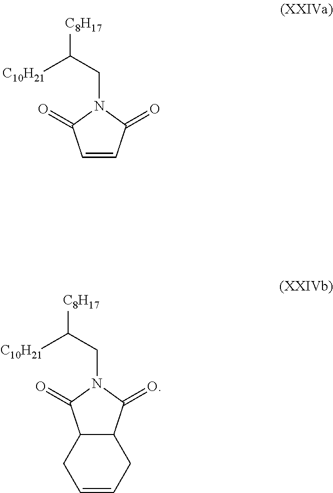

83. A method for synthesizing a compound having the following Formula (VI); 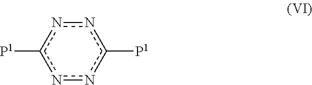
wherein P¹ has the following Formula (IIa) or (IIB); 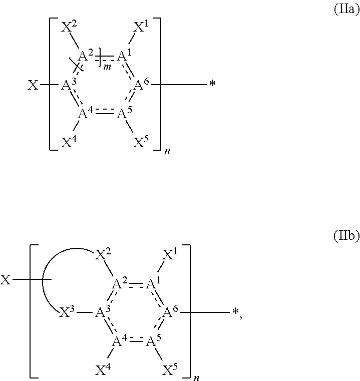
wherein [symbol]
is a single or double bond, as valency allows;
m is 0 or 1;
n is an integer selected from 1 to 4;
X, X¹, X², X³, X⁴ and X⁵ are independently absent or selected from the group consisting of a bond, H, halogen, CN, R¹, OR¹ or SR¹;
wherein R¹ is selected from the group consisting of optionally substituted alkyl, optionally substituted alkenyl, optionally substituted alkynyl, optionally substituted cycloalkyl, optionally substituted heterocycloalkyl, optionally substituted cycloalkenyl, optionally substituted heterocycloalkenyl, optionally substituted aryl, optionally substituted heteroaryl;
A¹ is selected from the group consisting of O, S, Se, Te, C and N;
A², A³, A⁴, A⁵ and A⁶ are independently C or N; and
wherein any two substituents selected from the group consisting of X¹, X², X³, X⁴ and X⁵ may optionally be taken together to form an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl, comprising providing a hydrazine and contacting the hydrazine with a functionalized cyclic group, wherein the cyclic group of the functionalized cyclic group is selected from the group comprising of optionally substituted furan, optionally substituted thiophene, optionally substituted selenophene, optionally substituted tellurophene, optionally substituted pyrrole, optionally substituted phenylene, optionally substituted aza-phenylene, optionally substituted arylene diimide, optionally substituted fluorene, optionally substituted cabazole, optionally substituted dibenzodiole, optionally substituted benzooxodiazole, optionally substituted benzotriazole, optionally substituted benzothidiazole and any combination thereof.

84. The method according to claim 83, wherein the functionalized cyclic group is functionalized with at least one nitrite group.

85. A method for synthesizing a compound having the following Formula (I); 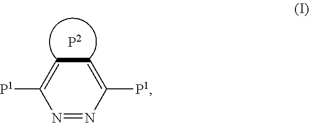
comprising:
providing a compound having the following Formula (VI); 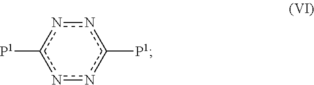
and
contacting a compound having the following Formula (VI); 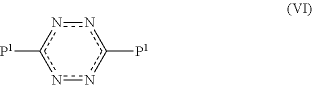
with a compound having the following formula (VIIa) or (VIIb); 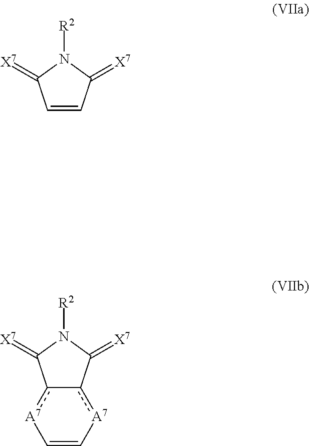
under reaction conditions to form a compound having the following Formula (I); 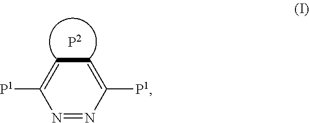
wherein P³ has the following Formula (IIa) or (IIb); 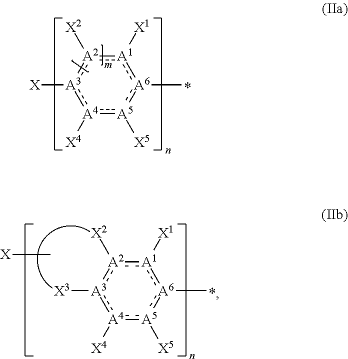
wherein [symbol]
is a single or double bond, as valency allows;
m is 0 or 1;
n is an integer selected from 1 to 4;
X, X¹, X², X³, X⁴ and X⁵ are independently absent or selected from the group consisting of a bond, H, halogen, CN, R¹, OR¹ or SR¹;
wherein R¹ is selected from the group consisting of optionally substituted alkyl, optionally substituted alkenyl, optionally substituted alkynyl, optionally substituted cycloalkyl, optionally substituted heterocycloalkyl, optionally substituted cycloalkenyl, optionally substituted heterocycloalkenyl, optionally substituted aryl, optionally substituted heteroaryl,
A¹ is selected from the group consisting of O, S, Se, Te, C and N;
A², A³, A⁴, A⁵ and A⁶ are independently C or N; and
wherein any two substituents selected from the group consisting of X¹, X², X³, X⁴ and X⁵ may optionally be taken together to form an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl;
and wherein P² has the following formula (IIIa) or (IIIb); 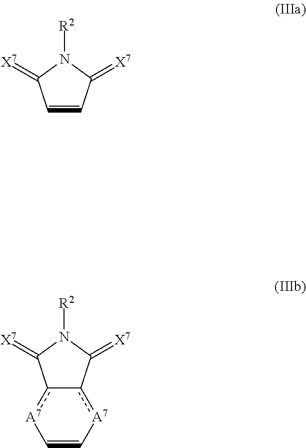
wherein
R² is an optionally substituted alkyl or optionally substituted aryl;
X⁷ is O or S;
A⁷ is CH, CH₂ or N;
and [symbol] is where the compound of Formula (IIIa) or (IIIb) is fused to the compound of Formula (I);
wherein [symbol]
is a single or double bond, as valency allows;
R² is an optionally substituted alkyl or optionally substituted aryl;
X⁷ is O or S; and
A⁷ is CH, CH₂ or N.

86. The method according to claim 85, further comprising reacting a compound having repeating units of the following Formula (IV); 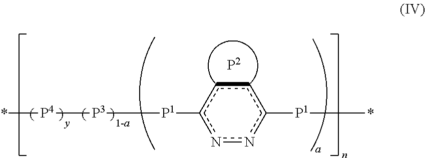
wherein P¹ has the following Formula (IIa) or (IIb); 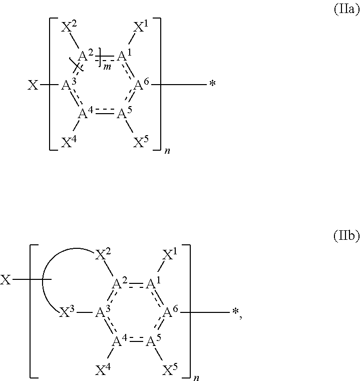
wherein [symbol]
is a single or double bond, as valency allows;
m is 0 or 1;
n is an integer selected from 1 to 4;
X¹, X², X³, X⁴ and X⁵ are independently absent or selected from the group consisting of a bond, H, halogen, CN, R¹, OR¹ or SR¹;
wherein R¹is selected from the group consisting of optionally substituted alkyl, optionally substituted alkenyl, optionally substituted alkynyl, optionally substituted cycloalkyl, optionally substituted heterocycloalkyl, optionally substituted cycloalkenyl, optionally substituted heterocycloalkenyl, optionally substituted aryl, optionally substituted heteroaryl,
A¹ is selected from the group consisting of O, S, Se, Te, C and N;
A², A³, A⁴, A⁵ and A⁶ are independently C or N; and
wherein any two substituents selected from the group consisting of X¹, X², X³, X⁴ and X⁵ may optionally be taken together to form an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl;
P² has the following formula (IIIa) or (IIIb); 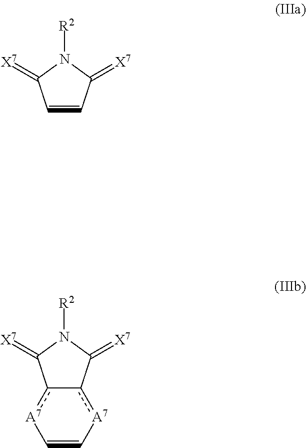
wherein
R² is an optionally substituted alkyl or optionally substituted aryl;
X⁷ is O or S;
A⁷ is CH, CH₂ or N;
and [symbol] is where the compound of Formula (IIIa) or (IIIb) is fused to the compound of Formula (IV);
P³ has the following Formula (Va), (Vb) or (Vc); 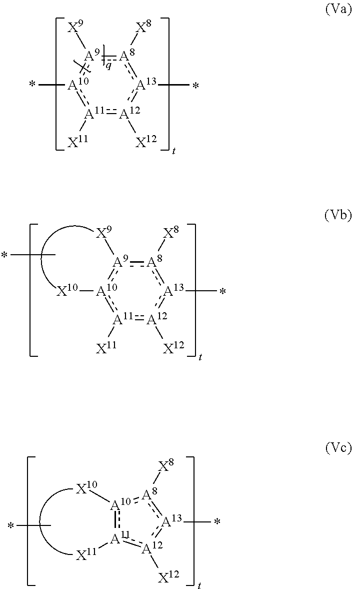
wherein
q is 0 or 1;
t is an integer selected from 1 to 4;
X⁸, X⁹, X¹¹ and X¹² are independently absent or selected from the group consisting of a bond, H, halogen, CN, R³, OR³ or SR³;
wherein R³ is selected from the group consisting of optionally substituted alkyl, optionally substituted alkenyl, optionally substituted alkynyl, optionally substituted cycloalkyl, optionally substituted heterocycloalkyl, optionally substituted cycloalkenyl, optionally substituted heterocycloalkenyl, optionally substituted aryl, optionally substituted heteroaryl,
A⁸ is selected from the group consisting of O, S, Se, Te, C and N
A⁹, A¹⁰, A¹¹, A¹² and A¹³ are independently C or N;
wherein any two substituents selected from the group consisting of X⁸, X⁹, X¹⁰, X¹¹ and X¹² may optionally be taken together to form an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl;
and P⁴ is absent, or is an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl,
a is any value from 0 to 1; and
b is an integer selected from 1 to 10,000; and
y is an integer selected from 0 to 5, with N-bromosuccinimide and hydrogen peroxide, followed by a base.

87. A method for synthesizing a compound having repeating units of the following Formula (IV); 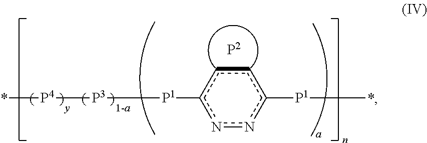
comprising:
providing a compound having the following Formula (I); 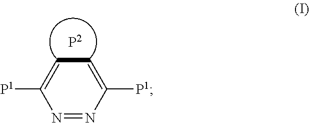
and
contacting a compound having the following Formula (I); 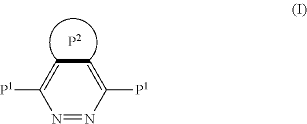
with;
(i) a compound having the following Formula (XXVa), (XXVb) or (XXVc); 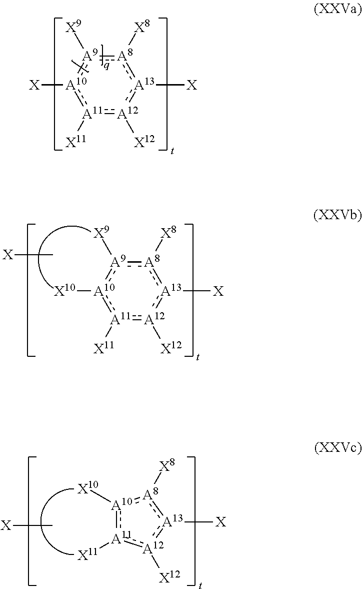
wherein [symbol]
is a single or double bond, as valency allows;
q is 0 or 1;
t is an integer selected from 1 to 4;
X, X⁸, X⁹, X¹¹ and X¹² are independently absent or selected from the group consisting of a bond, H, halogen, CN, R³, OR³or SR³;
wherein R³ is selected from the group consisting of optionally substituted alkyl, optionally substituted alkenyl, optionally substituted alkynyl, optionally substituted cycloalkyl, optionally substituted heterocycloalkyl, optionally substituted cycloalkenyl, optionally substituted heterocycloalkenyl, optionally substituted aryl, optionally substituted heteroaryl,
A⁸ is selected from the group consisting of O, S, Se, Te, C and N
A⁹, A¹⁰, A¹¹, A¹² and A¹³ are independently C or N; and
wherein any two substituents selected from the group consisting of X⁸, X⁹, X¹⁰, X¹¹ and X¹² may optionally be taken together to form an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl; and
(ii) a cross-coupling reagent comprising a cyclic group; and an element selected from the group consisting of zinc, tin and boron; wherein the cyclic group is selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl;
in the presence of a metal-containing catalyst;
wherein P¹ has the following Formula (IIa) or (IIb); 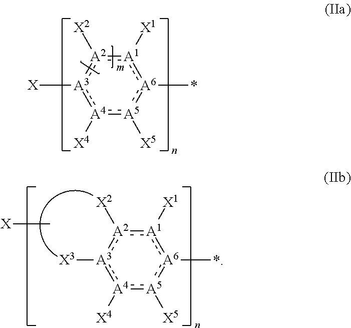
wherein [symbol]
is a single or double bond, as valency allows;
m is 0 or 1;
n is an integer selected from 1 to 4;
X¹, X², X³, X⁴ and X⁵ are independently absent or selected from the group consisting of a bond, H, halogen, CN, R¹OR¹ or SR¹;
wherein R¹ is selected from the group consisting of optionally substituted alkyl, optionally substituted alkenyl, optionally substituted alkynyl, optionally substituted cycloalkyl, optionally substituted heterocycloalkyl, optionally substituted cycloalkenyl, optionally substituted heterocycloalkenyl, optionally substituted aryl, optionally substituted heteroaryl,
A¹ is selected from the group consisting of O, S, Se, Te, C and N
A², A³, A⁴, A⁵ and A⁶ are independently C or N; and
wherein any two substituents selected from the group consisting of X¹, X², X³, X⁴ and X⁵ may optionally be taken together to form an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl;
P² has the following formula (IIIa) or (IIIb); 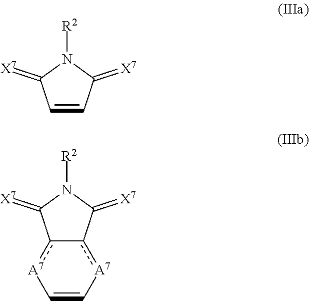
wherein
R² is an optionally substituted alkyl or optionally substituted aryl;
X⁷ is O or S;
A⁷ is CH, CH₂ or N;
and [symbol] is where the compound of Formula (IIIa) or (IIIb) is fused to the compound of Formula (IV);
P³ has the following Formula (Va), (Vb) or (Vc); 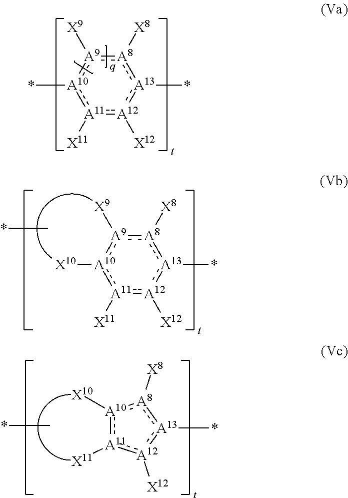
wherein
q is 0 or 1;
t is an integer selected from 1 to 4;
X⁸, X⁹, X¹¹ and X¹² are independently absent or selected from the group consisting of a bond, H, halogen, CN, R³, OR³ or SR³;
wherein R³ is selected from the group consisting of optionally substituted alkyl, optionally substituted alkenyl, optionally substituted alkynyl, optionally substituted cycloalkyl, optionally substituted heterocycloalkyl, optionally substituted cycloalkenyl, optionally substituted heterocycloalkenyl, optionally substituted aryl, optionally substituted heteroaryl,
A⁸ is selected from the group consisting of O, S, Se, Te, C and N
A⁹, A¹⁰, A¹¹, A¹² and A¹³ are independently C or N;
wherein any two substituents selected from the group consisting of X⁸, X⁹, X¹⁰, X¹¹ and X¹² may optionally be taken together to form an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl;
and P⁴ is absent, or is an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl,
a is any value from 0 to 1; and
b is an integer selected from 1 to 10,000; and
y is an integer selected from 0 to 5.

88. A method for synthesizing a compound having repeating units of the following Formula (IV); 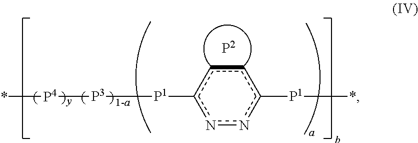
comprising:
1) providing a compound having the following Formula (I); 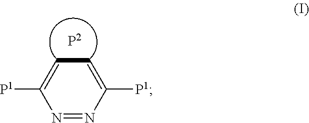
and
2) contacting a compound having the following Formula (I); 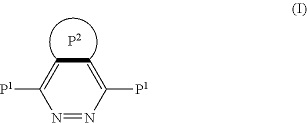
with a compound having the following Formula (XXVI); 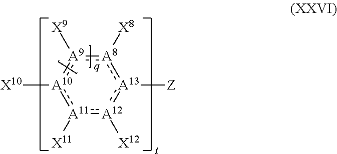
wherein [symbol]
is a single or double bond, as valency allows;
q is 0 or 1;
t is an integer selected from 1 to 4;
Z is a functional group containing zinc, tin or born;
X⁸, X⁹, X¹⁰, X¹¹ and X¹² are independently absent or selected from the group consisting of a bond, H, halogen, CN, R³, OR³ or SR³;
wherein R³ is selected from the group consisting of optionally substituted alkyl, optionally substituted alkenyl, optionally substituted alkynyl, optionally substituted cycloalkyl, optionally substituted heterocycloalkyl, optionally substituted cycloalkenyl, optionally substituted heterocycloalkenyl, optionally substituted aryl, optionally substituted heteroaryl,
A⁸ is selected from the group consisting of O, S, Se, Te, C and N
A⁹, A¹⁰, A¹¹, A¹² and A¹³ are independently C or N; and
wherein any two substituents selected from the group consisting of X⁸, X⁹, X¹⁰, X¹¹ and X¹² may optionally be taken together to form an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl;
in the presence of a metal-containing catalyst to form a monomer precursor;
3) reacting the monomer precursor of operation (2) with N-bromosuccinimide to form a reactive monomer; and
4) reacting the reactive monomer of operation (2) with a cross-coupling reagent comprising a cyclic group; and an element selected from the group consisting of zinc, tin and boron; wherein the cyclic group is selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl;
in the presence of a metal-containing catalyst;
wherein P¹ has the following Formula (IIa) or (IIb); 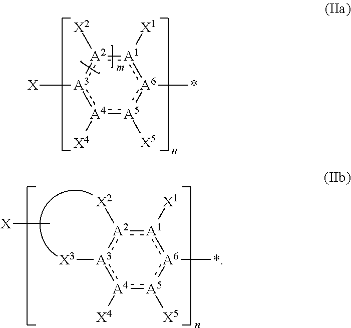
wherein [symbol]
is a single or, double bond, as valency allows;
m is 0 or 1;
n is an integer selected from 1 to 4;
X¹, X², X³, X⁴ and X⁵ are independently absent or selected from the group consisting of a bond, H, halogen, CN, R¹, OR¹ or SR¹;
wherein R¹ is selected from the group consisting of optionally substituted alkyl, optionally substituted alkenyl, optionally substituted alkynyl, optionally substituted cycloalkyl, optionally substituted heterocycloalkyl, optionally substituted cycloalkenyl, optionally substituted heterocycloalkenyl, optionally substituted aryl, optionally substituted heteroaryl,
A¹ is selected from the group consisting of O, S, Se, Te, C and N
A², A³, A⁴, A⁵ and A⁶ are independently C or N; and
wherein any two substituents selected from the group consisting of X¹, X², X³, X⁴ and X⁵ may optionally be taken together to form an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl;
P² has the following formula (IIIa) or (IIIb); 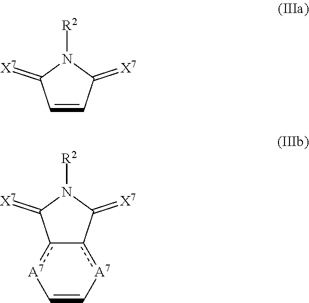
wherein
R² is an optionally substituted alkyl or optionally substituted aryl;
X⁷ is O or S;
A⁷ is CH, CH₂ or N;
and [symbol] is where the compound of Formula (IIIa) or (IIIb) is fused to the compound of Formula (IV);
P³ has the following Formula (Va), (Vb) or (Vc); 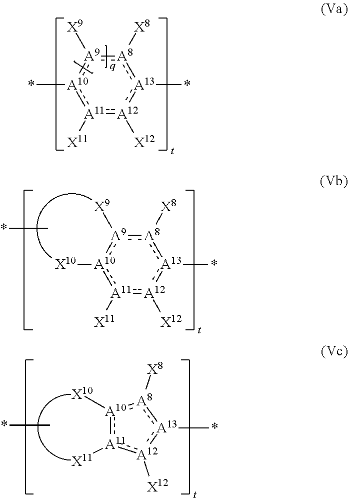
wherein
q is 0 or 1;
t is an integer selected from 1 to 4;
X⁸, X⁹, X¹¹ and X¹² are independently absent or selected from the group consisting of a bond, H, halogen, CN, R³, OR³ or SR³;
wherein R³ is selected from the group consisting of optionally substituted alkyl, optionally substituted alkenyl, optionally substituted alkynyl, optionally substituted cycloalkyl, optionally substituted heterocycloalkyl, optionally substituted cycloalkenyl, optionally substituted heterocycloalkenyl, optionally substituted aryl, optionally substituted heteroaryl,
A⁸ is selected from the group consisting of O, S, Se, Te, C and N
A⁹, A¹⁰, A¹¹, A¹² and A¹³ are independently C or N;
wherein any two substituents selected from the group consisting of X⁸, X⁹, X¹⁰, X¹¹ and X¹² may optionally be taken together to form an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl;
and P⁴ is absent, or is an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl,
a is any value from 0 to 1; and
b is an integer selected from 1 to 10,000; and
y is an integer selected from 0 to 5.

89. The method of claim 88, wherein the metal in the metal-containing catalyst is selected from the group consisting of palladium, nickel and copper.

90. (canceled)

91. A semiconductor device comprising a compound having repeating units of the following Formula (IV); 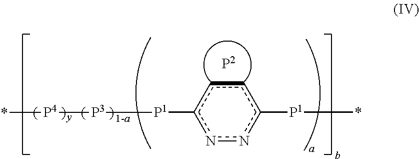
wherein P¹ has the following Formula (IIa) or (IIb); 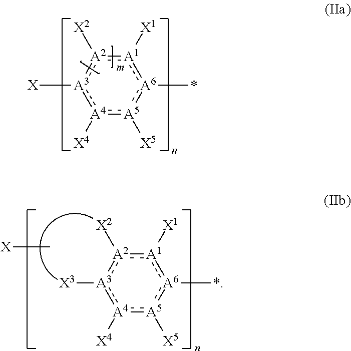
wherein [symbol]
is a single or double bond, as valency allows;
m is 0 or 1;
n is an integer selected from 1 to 4;
X¹, X², X³, X⁴ and X⁵ are independently absent or selected from the group consisting of a bond, H, halogen, CN, R¹, OR¹ or SR¹;
wherein R¹ is selected from the group consisting of optionally substituted alkyl, optionally substituted alkenyl, optionally substituted alkynyl, optionally substituted cycloalkyl, optionally substituted heterocycloalkenyl, optionally substituted cycloalkenyl, optionally substituted heterocycloalkenyl, optionally substituted, aryl, optionally substituted heteroaryl,
A¹ is selected from the group consisting of O, S, Se, Te, C and N
A², A³, A⁴, A⁵ and A⁶ are independently C or N; and
wherein any two substituents selected from the group consisting of X¹, X², X³, X⁴ and X⁵ may optionally be taken together to form an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl;
P² has the following formula (IIIa) or (IIIb); 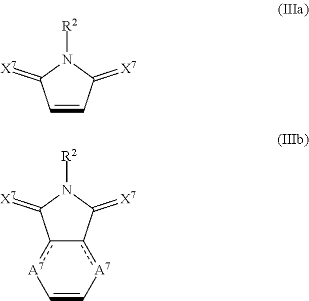
wherein
R² is an optionally substituted alkyl or optionally substituted aryl;
X⁷ is O or S;
A⁷ is CH, CH₂ or N;
and [symbol] is where the compound of Formula (IIIa) or (IIIb) is fused to the compound of Formula (IV);
P³ has the following Formula (Va), (Vb) or (Vc); 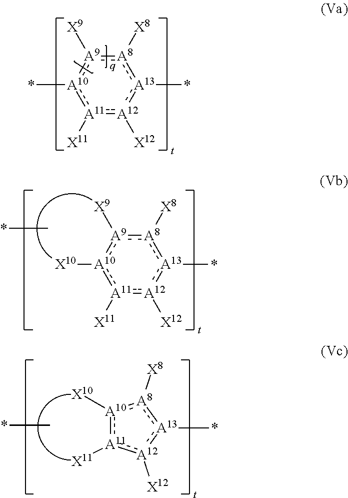
wherein
q is 0 or 1;
t is an integer selected from 1 to 4;
X⁸, X⁹, X¹¹ and X¹² are independently absent or selected from the group consisting of a bond, H, halogen, CN, R³, OR³ or SR³;
wherein R³ is selected from the group consisting of optionally substituted alkyl, optionally substituted alkenyl, optionally substituted alkynyl, optionally substituted cycloalkyl, optionally substituted heterocycloalkyl, optionally substituted cycloalkenyl, optionally substituted heterocycloalkenyl, optionally substituted aryl, optionally substituted heteroaryl,
A⁸ is selected from the group consisting of O, S, Se, Te, C and N
A⁹, A¹⁰, A¹¹, A¹² and A¹³ are independently C or N;
wherein any two substituents selected from the group consisting of X⁸, X⁹, X¹⁰, X¹¹ and X¹² may optionally be taken together to form an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl;
and P⁴ is absent, or is an optionally substituted cyclic group selected from the group consisting of an optionally substituted cycloalkyl, optionally substituted heterocycloalkyl, optionally substituted cycloalkenyl, optionally substituted heterocycloalkenyl, optionally substituted aryl and optionally substituted heteroaryl,
a is any value from 0 to 1; and
b is an integer selected from 1 to 10,000; and
y is an integer selected from 0 to 5.
